SEQUENCE LISTING (16 sequences)

SEQ ID NO: 1 - DNA, 1302 bp, Artificial Sequence
  Nucleotide sequence of 4G8 FLT3 CAR
caggtgcagctgcagcagcctggcgccgaactcgtgaaacctggcgcctctctgaagctgagctgcaagagcagcggctacaccttcaccagctactggatgcactgggtgcgccagaggcctggccacggactggaatggatcggcgagatcgaccccagcgacagctacaaggactacaaccagaagttcaaggacaaggccaccctgaccgtggacagaagcagcaacaccgcctacatgcacctgtccagcctgaccagcgacgacagcgccgtgtactactgtgccagagccatcacaaccacccccttcgatttctggggccagggcacaaccctgacagtgtctagcggaggcggaggctccggagggggaggatctgggggaggcggaagcgatattgtgctgacccagagccctgccacactgagcgtgacaccaggcgatagcgtgtccctgtcctgcagagccagccagagcatctccaacaacctgcactggtatcagcagaagtcccacgagagccccagactgctgattaagtacgccagccagtccatcagcggcatccccagcagattttccggcagcggctccggcaccgacttcaccctgagcatcaacagcgtggaaaccgaggacttcggcgtgtacttctgccagcagagcaacacctggccttacaccttcggcggaggcaccaagctggaaatcaagagagagtctaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 2 - protein, 434 aa, Artificial Sequence
  Amino acid sequence of 4G8 FLT3 CAR
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSGGGGSGGGGSGGGGSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKRESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1335 bp, Artificial Sequence
  Nucleotide sequence of BV10 FLT3 CAR
caggtgcagctgaagcagagcggccctggactggtgcagcctagccagagcctgagcatcacctgtaccgtgtccggcttcagcctgaccaactacggcctgcattgggtgcgccagagccctggcaaaggcctggaatggctgggagtgatttggagcggcggcagcaccgactacaacgccgccttcatcagcagactgagcatctccaaggacaacagcaagagccaggtgttcttcaagatgaactccctgcaggccgacgacaccgccatctactactgcgccagaaagggcggcatctactatgccaaccactactacgctatggactactggggccagggcaccagcgtgacagtgtctagcggaggcggaggctccggagggggaggatctgggggaggcggatctgacatcgtgatgacccagagccccagcagcctgtctgtgtctgccggcgagaaagtgaccatgagctgcaagagcagccagtccctgctgaacagcggcaaccagaaaaactacatggcctggtatcagcagaagcccggccagccccctaagctgctgatctacggcgccagcaccagagaaagcggcgtgcccgatagattcaccggcagcggctctggcaccgactttaccctgaccatcagcagcgtgcaggctgaggacctggccgtgtactactgccagaacgaccacagctaccccctgacctttggagccggcaccaagctggaactgaagagagagtctaagtacggaccgccctgccccccttgccctatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 4 - protein, 445 aa, Artificial Sequence
  Amino acid sequence of BV10 FLT3 CAR
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSGGGGSGGGGSGGGGSDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKRESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 118 aa, Artificial Sequence
  4G8 FLT3 CAR 4G8 heavy chain variable domain (VH)
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 6 - protein, 108 aa, Artificial Sequence
  4G8 FLT3 CAR 4G8 light chain variable domain (VL)
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 7 - protein, 123 aa, Artificial Sequence
  BV10 FLT3 CAR BV10 heavy chain variable domain (VH)
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSS

SEQ ID NO: 8 - protein, 114 aa, Artificial Sequence
  BV10 FLT3 CAR BV10 light chain variable domain (VL)
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 9 - protein, 22 aa, Artificial Sequence
  CAR GMCSF signal peptide
MLLLVTSLLLCELPHPAFLLIP

SEQ ID NO: 10 - protein, 15 aa, Artificial Sequence
  CAR 4(GS)x3 linker
GGGGSGGGGSGGGGS

SEQ ID NO: 11 - protein, 12 aa, Artificial Sequence
  CAR IgG4 hinge domain
ESKYGPPCPPCP

SEQ ID NO: 12 - protein, 28 aa, Artificial Sequence
  CAR CD28 transmembrane domain
MFWVLVVVGGVLACYSLLVTVAFIIFWV

SEQ ID NO: 13 - protein, 41 aa, Artificial Sequence
  CAR CD28 costimulatory domain
RSKRSRGGHSDYMNMTPRRPGPTRKHYQPYAPPRDFAAYRS

SEQ ID NO: 14 - protein, 112 aa, Artificial Sequence
  CAR CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 24 aa, Artificial Sequence
  CAR T2A ribosomal skipping sequence
LEGGGEGRGSLLTCGDVEENPGPR

SEQ ID NO: 16 - protein, 335 aa, Artificial Sequence
  CAR EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

(19) United States
(12) Patent Application Publication
(10) Pub. No.: US 2020/0206266 A1
(43) Pub. Date: Jul. 2, 2020

(54) USE OF FLT3 CAR-T CELLS AND FLT3 INHIBITORS TO TREAT ACUTE MYELOID LEUKEMIA

(71) Applicant: JULIUS-MAXIMILIANS-UNIVERSITÄT WÜRZBURG, Würzburg (DE)

(72) Inventors: Michael HUDECEK, Höchberg (DE); Hardikkumar JETANI, Würzburg (DE)

(21) Appl. No.: 16/633,132

(22) PCT Filed: Aug. 1, 2018
(86) PCT No.: PCT/EP2018/070856

§ 371 Date: Jan. 22, 2020

(30) Foreign Application Priority Data
Aug. 1, 2017 (EP) 17184277.6

(51) Int. Cl. A61K 35/17; A61K 31/4709; C07K 16/28; A61P 35/00

(52) U.S. Cl. CPC A61K 35/17; A61P 35/00; C07K 16/2863; A61K 31/4709

(57) ABSTRACT

The invention generally relates to the treatment of cancer with FLT3 targeting agents and kinase inhibitors. In particular, the invention relates to adoptive immunotherapy of Acute Myeloid Leukemia (AML) with chimeric antigen receptor (CAR)-modified T cells specific for FMS-like tyrosine kinase (FLT3) in combination with FLT3 inhibitors.

### FIELD OF THE INVENTION

The invention generally relates to the treatment of cancer with FLT3 targeting agents and kinase inhibitors. In particular, the invention relates to adoptive immunotherapy of Acute Myeloid Leukemia (AML) with chimeric antigen receptor (CAR)-modified T cells specific for FMS-like tyrosine kinase (FLT3) in combination with FLT3 inhibitors.

### BACKGROUND OF THE INVENTION

FMS-like tyrosine kinase 3 (FLT3) is a type I transmembrane protein that plays an essential role in normal hematopoiesis and is physiologically expressed on normal hematopoietic stem cells (HSCs), as well as lymphoid, myeloid and granulocyte/macrophage progenitor cells in humans¹⁻⁴. In mature hematopoietic cells, FLT3 expression has been reported in subsets of dendritic cells and natural killer cells⁵⁻⁷. FLT3 is also uniformly present on malignant blasts in acute myeloid leukemia (AML), providing a target for antibody and cellular immunotherapy^{1, 4,8-11}. The antigen density of FLT3 protein on the cell surface of AML blasts is in the range of several hundred to several thousand molecules per cell, which is optimal for recognition by engineered T cells that are equipped with a synthetic chimeric antigen receptor (CAR)^{12,13}.

At the molecular level, FLT3 transcripts are universally detectable in AML blasts, with graded expression levels in distinct FAB (French-American-British) subtypes^{9, 14}. Higher FLT3 transcript levels correlate with higher leukocyte counts and higher degrees of bone marrow infiltration by leukemic cells, independent from the presence of FLT3 mutations¹¹. FLT3 is important for survival and proliferation of AML blasts and of particular pathophysiologic relevance in AML cases that carry activating mutations in the FLT3 intracellular domain^{5, 11}. Of these, internal tandem duplications (ITDs) in the juxtamembrane domain and mutations in the intracellular tyrosine kinase domain (TKD) are the most common aberrations that collectively occur in approx. 30% of AML cases^{1, 11, 14, 15}. Both aberrations cause constitutive FLT3 activation in a ligand-independent manner and act as gain-of-function ‘driver mutations’ that contribute to sustaining the malignant disease¹⁶⁻¹⁸. These attributes suggest FLT3-ITD⁺ AML is particularly susceptible and indeed a preferred AML subset for anti-FLT3 immunotherapy because the risk to incur FLT3^{-/low} antigen-loss AML blast variants is anticipated to be low. Indeed, the presence of an FLT3-ITD is associated with an inferior clinical outcome after induction/consolidation chemotherapy and allogeneic hematopoietic stem cell transplantation (HSCT), and defines a subset of high-risk AML patients that require novel, innovative treatment strategies^{19, 20}.

FLT3 is being pursued as a target for tyrosine kinase inhibitors and numerous substances are at advanced stages of clinical development. However, the clinical efficacy of single agent therapy with ‘first-generation’ FLT3 inhibitors has been rather limited, owing at least in part to the development of resistance through novel mutations in the FLT3 intracellular domain, or FLT3 overexpression in AML blasts²¹⁻²⁵.

Monotherapy using TKI may result in measurable clinical response including significant reductions of peripheral blood (PB) and bone marrow (BM) blasts. However, in most cases patients become resistant after transient responses known as secondary resistance development. The emergence of novel mutations in tyrosine kinase and/or juxtamembrane domains after treatment with TKI (primary resistance) has been observed frequently which limits clinical activity of TKI in refractory and relapsed AML patient as a single agent therapy.

Midostaurin is a ‘first-generation’ FLT3 inhibitor and derivative of the alkaloid staurosporine and multi-kinase inhibitor. Midostaurin inhibits FLT3, platelet-derived growth factor receptors (PDGFRs) alpha and beta, cyclin-dependent kinase 1 (cdk1), src, Fgr, Syk (spleen tyrosine kinase), c-kit, and the major vascular endothelial growth factor (VEGF) receptor, KDR. Midostaurin is a type II FLT3 inhibitor and has shown activity against mutant FLT3 in vitro and in vivo (Ref.: #21-23).

Quizartinib (AC220) is a ‘first-generation’ FLT3 inhibitor drug designed specifically against FLT3. Quizartinib is a type II FLT3 inhibitor and has shown activity against FLT3-ITD⁺ AML. Quizartinib has shown significant improvement in overall survival in FLT3-ITD⁺ AML patients that relapsed after stem cell transplantation or after failure of salvage chemotherapy (Ref.: 21).

Crenolanib is a specific type-I-inhibitor that targets the active FLT3 kinase conformation and is effective against FLT3 with ITD and TKD mutations that confer resistance to type-II-inhibitors, e.g. midostaurin and quizartinib that target the inactive kinase conformation^{26, 27}.

Crenolanib is also active against platelet-derived growth factor receptor alpha/beta and is being evaluated in patients with gastrointestinal stromal tumors and gliomas^{28, 29}. In AML, crenolanib has proven effective in relapsed/refractory AML with FLT3-ITD and TKD mutations, with remarkable response rates in recently reported phase II clinical trials^{30, 31}. Crenolanib and other TKIs are therefore being investigated in combination regimens to enhance efficacy.

FLT3 has also been pursued as a target for antibody immunotherapy, even though the antigen density of FLT3 on AML blasts is much lower compared to e.g. CD20 on lymphoma cells and not presumed to be optimal for inducing potent antibody-mediated effector functions¹². A mouse anti-human FLT3 monoclonal antibody (mAb) 4G8 has been shown to specifically bind to AML blasts and to a lesser extent to normal HSCs—and to confer specific reactivity against AML blasts with high FLT3 antigen density in pre-clinical models after Fc-optimization¹⁴.

The inventors engineered T cells to express a FLT3-specific CAR with a targeting domain derived from the 4G8 mAb and analyze the antileukemia reactivity of FLT3 CAR-T cells against FLT3 wild-type and FLT3-ITD⁺ AML cells, alone and in combination with the FLT3 inhibitors midostaurin, quizartinib and crenolanib. Further, the inventors evaluate recognition of normal HSC as an anticipated side effect of effectively targeting FLT3 to identify clinical settings for adoptive immunotherapy with FLT3 CAR-T cells in the context of allogeneic HSCT.

### DESCRIPTION OF THE INVENTION

The invention generally relates to the treatment of cancer with FLT3 targeting agents, especially immunotherapeutic targeting agents, and kinase inhibitors. In particular, the invention relates to the treatment of Acute Myeloid Leukemia (AML), preferably with T cells that were modified by gene-transfer to express an FLT3-specific chimeric antigen receptor (CAR) in combination with FLT3 inhibitors. In the present invention, we demonstrate that treatment of AML blasts with FLT3 inhibitors leads to a significant increase in expression of the FLT3 molecule on the cell surface of AML blasts, which as a consequence leads to a significant increasing in recognition and elimination by FLT3 CAR-T cells. The combination treatment of AML with FLT3 targeting agents, in particular CAR-T cells, and kinase inhibitors, in particular FLT3 inhibitors, is highly synergistic and superior to monotherapy with either FLT3 inhibitors or FLT3 CAR-T cells alone.

The present invention is exemplified by the following preferred embodiments:
1. A composition for use in a method for the treatment of cancer in a patient, the composition comprising:
   (a) A kinase inhibitor; and
   (b) An FLT3-targeting agent;
wherein in the method, the composition is to be administered to the patient.
2. The composition of item 1 for the use of item 1, wherein the method is a method comprising adoptive immunotherapy.
3. The composition of items 1 or 2 for the use of items 1 or 2, wherein the FLT3-targeting agent is capable of binding to the extracellular domain of FLT3.
4. The composition of any of items 1 to 3 for the use of any of items 1 to 3, wherein the FLT3-targeting agent inhibits growth of cells expressing FLT3.
5. The composition of any of items 1 to 4 for the use of any of items 1 to 4, wherein the FLT3-targeting agent comprises a cell targeting FLT3.
6. The composition of item 5 for use of item 5, wherein the cell is a cell expressing a chimeric antigen receptor.
7. The composition of item 6 for use of item 6, wherein the chimeric antigen receptor is capable of binding to FLT3.
8. The composition of any of items 5 to 7 for use of any of items 5 to 7, wherein the cell is a cell selected from the group of T cells, NK cells, and B cells.
9. The composition of any of items 5 to 8 for use of any of items 5 to 8, wherein the cell is a T cell.
10. The composition of any of items 6 to 9 for use of any of items 6 to 9, wherein the chimeric antigen receptor comprises the sequence of SEQ ID NO: 2 or a sequence at least 90% identical thereto, or wherein the chimeric antigen receptor comprises the sequence of SEQ ID NO: 4 or a or a sequence at least 90% identical thereto.
11. The composition of item 10 for use of item 10, wherein the chimeric antigen receptor comprises the sequence of SEQ ID NO: 2, or a sequence at least 90% identical thereto.
12. The composition of item 10 for use of item 10, wherein the chimeric antigen receptor comprises the sequence of SEQ ID NO: 4, or a sequence at least 90% identical thereto.
13. The composition of any of items 6 to 12 for use of any of items 6 to 12, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto, or wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.
14. The composition of item 13 for use of item 13, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto.
15. The composition of item 13 for use of item 13, wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.
16. The composition of any of items 1 to 4 for the use of any of items 1 to 4, wherein the FLT3-targeting agent comprises a protein.
17. The composition of item 16 for the use of item 16, wherein the protein is an antibody or fragment thereof capable of binding to FLT3.
18. The composition of item 17 for the use of item 17, wherein the antibody or fragment thereof comprises a heavy chain variable domain sequence of SEQ ID NO: 5 or a sequence at least 90% identical thereto and a light chain variable domain sequence of SEQ ID NO: 6 or a sequence at least 90% identical thereto, or wherein the antibody or fragment thereof comprises a heavy chain variable domain sequence of SEQ ID NO: 7 or a sequence at least 90% identical thereto and a light chain variable domain sequence of SEQ ID NO: 8 or a sequence at least 90% identical thereto.
19. The composition of item 18 for the use of item 18, wherein the antibody is an antibody comprising a heavy chain variable domain which comprises the amino acid sequence of SEQ ID NO: 5, and a light chain variable domain which comprises the amino acid sequence of SEQ ID NO: 6.
20. The composition of item 18 for the use of item 18, wherein the antibody is an antibody comprising a heavy chain variable domain which comprises the amino acid sequence of SEQ ID NO: 7, and a light chain variable domain which comprises the amino acid sequence of SEQ ID NO: 8.
21. The composition of any of items 1 to 20 for the use of any of items 1 to 20, wherein the kinase inhibitor is a multikinase inhibitor.
22. The composition of any of items 1 to 21 for the use of any of items 1 to 21, wherein the kinase inhibitor is a tyrosine kinase inhibitor.
23. The composition of any of items 1 to 22 for the use of any of items 1 to 22, wherein the kinase inhibitor is an FLT3 inhibitor.
24. The composition of any of items 1 to 23 for the use of any of items 1 to 23, wherein the kinase inhibitor is a kinase inhibitor capable of causing upregulation of FLT3 in said cancer.
25. The composition of any of items 1 to 24 for the use of any of items 1 to 24, wherein the kinase inhibitor is a kinase inhibitor capable of causing upregulation of FLT3 cell surface expression in said cancer.
26. The composition of any of items 1 to 25 for the use of any of items 1 to 25, wherein the kinase inhibitor is a kinase inhibitor capable of causing upregulation of mutated FLT3 in said cancer.
27. The composition of any of items 1 to 26 for the use of any of items 1 to 26, wherein the kinase inhibitor does not cause upregulation of wild-type FLT3 in said cancer.
28. The composition of item 26 for the use of item 26, wherein the mutated FLT3 comprises a mutated tyrosine kinase domain, and/or wherein the mutated FLT3 comprises internal tandem duplications.
29. The composition of item 28 for the use of item 28, wherein the mutated FLT3 comprises internal tandem duplications.
30. The composition of item 28 for the use of items 28, wherein the mutated FLT3 comprises a mutated tyrosine kinase domain.
31. The composition of any of items 1 to 30 for the use of any of items 1 to 30, wherein the kinase inhibitor does not inhibit T cells expressing chimeric antigen receptors.
32. The composition of any of items 1 to 31 for the use of any of items 1 to 31, wherein the kinase inhibitor is a type I or a type II FLT3 inhibitor.
33. The composition of item 32 for the use of item 32, wherein the kinase inhibitor is a type I FLT3 inhibitor.
34. The composition of item 32 for the use of item 32, wherein the kinase inhibitor is a type II FLT3 inhibitor.
35. The composition of any of items 1 to 32 for the use of any of items 1 to 32, wherein the kinase inhibitor is selected from the group consisting of crenolanib, midostaurin, and quizartinib.
36. The composition of item 35 for the use of item 35, wherein the kinase inhibitor is crenolanib.
37. The composition of item 35 for the use of item 35, wherein the kinase inhibitor is quizartinib.
38. The composition of item 35 for the use of item 35, wherein the kinase inhibitor is midostaurin.
39. The composition of any of items 1 to 38 for the use of any of items 1 to 38, wherein said treatment of cancer has an improved clinical outcome compared to a monotherapeutic treatment with either said FLT3-targeting agent or said kinase inhibitor alone.
40. The composition of any of items 1 to 39 for the use of any of items 1 to 39, wherein the FLT3-targeting agent and the kinase inhibitor prolong the progression free survival of the patient compared to monotherapy with either said FLT3-targeting agent or said kinase inhibitor alone.
41. The composition of any of items 5 to 40 for the use of any of items 5 to 40, wherein the cell produces effector cytokines when administered to the patient.
42. The composition of item 41 for the use of item 41, wherein the cytokines are IFN-gamma and IL-2.
43. The composition of any of items 1 to 42 for the use of any of items 1 to 42, wherein said cancer is leukemia or lymphoma.
44. The composition of item 43 for the use of item 43, wherein said cancer is leukemia.
45. The composition of item 44 for the use of item 44, wherein said leukemia is mixed-lineage leukemia or acute lymphoblastic leukemia.
46. The composition of item 44 for the use of item 44, wherein said leukemia is acute myeloid leukemia.
47. The composition of any of items 1 to 46 for the use of any of items 1 to 46, wherein the method is a method wherein the number of FLT3 molecules on the cell surface is increased, preferably wherein the number of FLT3 molecules on the cell surface is increased in the cancer cells.
48. The composition of item 47 for the use of item 47, wherein the FLT3 upregulation is caused by treatment with said kinase inhibitor.
49. The composition of item 48 for the use of item 48, wherein the cancer has acquired a resistance to a monotherapeutic treatment with said kinase inhibitor or wherein the cancer has acquired a resistance to a monotherapeutic treatment with said kinase inhibitor in combination with chemotherapy.
50. The composition of any of items 1 to 49 for the use of any of items 1 to 49, wherein the cancer expresses wild-type FLT3.
51. The composition of any of items 1 to 49 for the use of any of items 1 to 49, wherein the cancer expressed mutated FLT3.
52. The composition of item 51 for the use of item 51, wherein the mutated FLT3 is mutationally activated.
53. The composition of any of items 51 or 52 for the use of any of items 51 or 52, wherein the mutated FLT3 is mutated in the tyrosine kinase domain.
54. The composition of any of items 51 to 53 for the use of any of items 51 to 53, wherein the mutated FLT3 comprises internal tandem duplications.
55. The composition of any of items 1 to 54 for the use of any of items 1 to 54, wherein the treatment is a first-line therapy.
56. The composition of any of items 1 to 54 for the use of any of items 1 to 54, wherein the treatment is a second-line therapy, a third-line therapy, or a fourth-line therapy.
57. A chimeric antigen receptor capable of binding FLT3.
58. The chimeric antigen receptor of item 57, wherein the chimeric antigen receptor comprises an IgG4-Fc hinge spacer, a CD28 transmembrane and costimulatory domain, and a CD3z signaling domain.
59. The chimeric antigen receptor of any of items 57 or 58, wherein the chimeric antigen receptor comprises the sequence of SEQ ID NO: 2 or a sequence at least 90% identical thereto, or wherein the chimeric antigen receptor comprises the sequence of SEQ ID NO: 4 or a sequence at least 90% identical thereto.
60. The chimeric antigen receptor of item 59, wherein the chimeric antigen receptor comprises the sequence of SEQ ID NO: 2 or a sequence at least 90% identical thereto.
61. The chimeric antigen receptor of item 59, wherein the chimeric antigen receptor comprises the sequence of SEQ ID NO: 4 or a sequence at least 90% identical thereto.
62. The chimeric antigen receptor of any of items 57 or 58, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto, or wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.
63. The chimeric antigen receptor of item 62, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto.
64. The chimeric antigen receptor of item 62, wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.
65. A cell comprising the chimeric antigen receptor of any one of items 57 to 64.
66. The cell of item 65, wherein the cell expressing the chimeric antigen receptor is obtainable by expressing the chimeric antigen receptor through stable gene transfer.
67. The cell of item 65, wherein the cell expressing the chimeric antigen receptor is obtainable by expressing the chimeric antigen receptor through transient gene transfer.
68. The cell of any of items 65 to 67, wherein the cell is a cell selected from the group of T cells, NK cells, and B cells.
69. The cell of item 68, wherein the cell is a T cell.
70. The cell of any of items 65 to 69, wherein the cell is CD8 positive.
71. The cell of any of items 65 to 70, wherein the cell is CD4 positive.
72. An FLT3-targeting agent for use in a method of treating cancer.
73. The FLT3-targeting agent of item 72 for the use of item 72, wherein the method of treating cancer is a method of treating cancer with a kinase inhibitor.
74. The FLT3-targeting agent of any of items 72 or 73 for the use of any of items 72 or 73, wherein the FLT3-targeting agent is an FLT3-targeting agent as defined in any one of items 3 to 20.
75. The FLT3-targeting agent of any of items 72 to 74 for the use of any of items 72 to 74, wherein the kinase inhibitor is a kinase inhibitor as defined in any one of items 21 to 38.
76. The FLT3-targeting agent of any of items 72 to 75 for the use of any of items 72 to 75, wherein the cancer is a cancer as defined any one of items 43-54.
77. The FLT3-targeting agent of any of items 72 to 76 for the use of any of items 72 to 76, wherein the use is a use as defined in any one of items 1-56.
78. The FLT3-targeting agent of any of items 72 to 77 for the use of any of items 72 to 77, wherein the kinase inhibitor is to be administered at least once or multiple times prior to administering the FLT3-targeting agent, concurrently to administering the FLT3-targeting agent, or after administering the FLT3-targeting agent.
79. The FLT3-targeting agent of item 78 for the use of item 78, wherein the kinase inhibitor is to be administered at least once or multiple times prior to administering the FLT3-targeting agent.
80. The FLT3-targeting agent of item 78 for the use of item 78, wherein the kinase inhibitor is to be administered at least once or multiple times concurrently to administering the FLT3-targeting agent.
81. The FLT3-targeting agent of item 78 for the use of item 78, wherein the kinase inhibitor is to be administered at least once or multiple times after administering the FLT3-targeting agent.
82. A kit comprising an FLT3-targeting agent and a kinase inhibitor.
83. The kit according to item 82, wherein the FLT3-targeting agent is an FLT3-targeting agent as defined in any one of items 3-20.
84. The kit according to any of items 82 or 83, wherein the kinase inhibitor is a kinase inhibitor as defined in any one of items 21-38.
85. The kit according to any of items 82 to 84, wherein said FLT3-targeting agent further comprises a pharmaceutical acceptable carrier.
86. The kit according to any of items 82 to 85, wherein said kinase inhibitor further comprises a pharmaceutical acceptable carrier.
87. A composition comprising:
   (a) A kinase inhibitor; and
   (b) An FLT3-targeting agent.
88. The composition of item 87, wherein the FLT3-targeting agent is an FLT3-targeting agent as defined in any one of items 3-20.
89. The composition of any of items 87 or 88, wherein the kinase inhibitor is kinase inhibitor as defined in any one of items 21-38.
90. The composition of any of items 87 to 89, further comprising a pharmaceutically acceptable carrier.
91. The composition of any of items 87 to 90, wherein the composition is suitable for treating cancer.
92. The composition of item 91, wherein the cancer is a cancer as defined in any one of items 43-54.
93. A combination of the FLT3-targeting agent as defined in item 72 and a kinase inhibitor.
94. The combination of item 93 for use in a method for the treatment of cancer in a patient.
95. The combination of item 93 or the combination for use of item 94, wherein the FLT3-targeting agent is an FLT3-targeting agent as defined in any one of items 3-20.
96. The combination of item 93 or the combination for use of any of items 94 to 95, wherein the kinase inhibitor is kinase inhibitor as defined in any one of items 21-38.
97. The combination of item 93 or the combination for use of any of items 94 to 96, wherein the cancer is a cancer as defined in any one of items 43-54.
98. The combination of item 93 or the combination for use of any of items 94 to 97, wherein the use is a use as defined in any one of items 1-56.
99. A combination of FLT3 CAR-T cells and a kinase inhibitor, for use in a method for the treatment of cancer, wherein the combination is to be administered prior to or after an allogeneic hematopoietic stem cell transplantation to treat the cancer.
100. The combination for use according to item 99, wherein the FLT3 CAR-T cells are autologous FLT3 CAR-T cells.
101. The combination for use according to item 99, wherein the FLT3 CAR-T cells are allogeneic FLT3 CAR-T cells.
102. The combination for use according to any one of items 99 to 101, wherein the cancer is a cancer as defined in any one of items 43-54.
103. The combination for use according to any one of items 99 to 102, wherein the cancer is FLT3-ITD+AML.
104. The combination for use according to any one of items 99 to 103, wherein the kinase inhibitor is as defined in any one of items 21-38.
105. The combination for use according to any one of items 99 to 104, wherein the kinase inhibitor is crenolanib.

In a preferred embodiment, the chimeric antigen receptor in accordance with the invention comprises a costimulatory domain capable of mediating costimulation to immune cells.

The costimulatory domain is preferably from 4-1BB, CD28, Ox40, ICOS or DAP10.

The chimeric antigen receptor according to the invention further comprises a transmembrane domain, which is preferably a transmembrane domain from CD4, CD8 or CD28.

The chimeric antigen receptor according the invention preferably further comprises a CAR spacer domain, wherein said CAR spacer domain is preferably from CD4, CD8, an FC-receptor, an immunoglobulin, or an antibody.

### BRIEF DESCRIPTION OF THE DRAWINGS

FIG. 1: FLT3 CAR construct. Construction of FLT3 CARs, CD19 CAR and CD123 CAR used in the study. Single chain variable fragment (scFv) antigen-binding domains were derived from mAbs 4G8 and BV10 (FLT3 CARs), FMC63 (CD19 CAR), and 32716 (CD123 CAR). The scFv domains were linked via IgG4 hinge spacer and CD28 transmembrane domain to the intracellular domain. CD28 and CD3z were incorporated as costimulatory and signaling domains, respectively. Truncated epidermal growth factor receptor (tEGFR) (separated from CAR transgene by T2A ribosomal skip sequence) was incorporated for detection and enrichment of CAR-positive cells.

FIG. 2: Phenotype of FLT3 CAR-T cells. T cells isolated from healthy donors or AML patients peripheral blood mononuclear cells were stimulated with CD3/CD28 beads, CAR transgene was lentivirally transduced, stained (after 8-10 days) with biotinylated anti-tEGFR antibody followed by anti-biotin magnetic beads staining and sorted using Magnetic-Activated Cell Sorting (MACS). Flow cytometric analysis of CAR expression by CD8⁺ and CD4⁺ T cells after MACS sorting.

FIG. 3: FLT3 CAR-T cells specifically recognize FLT3-transduced K562 tumor cells. K562/FLT3 was generated by retroviral transduction with the full-length human FLT3 gene. (a) Flow cytometric analysis of FLT3 expression by K562 native and K562/FLT3 cells. (b) Specific cytolytic activity of CD8⁺ FLT3 CAR-T cells, analyzed after 4-hour in a bioluminescence-based cytotoxicity assay. Values are presented as mean+s.d. The right-hand graph shows cytolytic activity of CAR T cells prepared from three different T cell donors.

FIG. 4: FLT3 CAR-T cells recognize and eliminate FLT3 wild-type and FLT3-ITD⁺ AML cell lines and primary AML cells in vitro. (a) Flow cytometric analysis of FLT3 expression on AML cell lines (MOLM-13, THP-1, MV4;11) and primary AML blasts (pt #1 and #2). Histograms show staining with anti-FLT3 mAb (4G8) (solid line) and isotype control antibody (zebra line). ΔMFI (Difference in mean fluoresence intensity) values represents absolute difference in MFI of anti-FLT3 mAb stained and isotype control stained cells. (b) Specific cytolytic activity of CD8⁺ FLT3 CAR-T cells, CD19 CAR-T cells or untransduced T cells (UTD) against AML cell lines analyzed after 4-hour in a bioluminescence-based cytotoxicity assay. Assay was performed in triplicate wells at the indicated effector to target cell ratio with 5,000 target cells/well. Values are presented as mean+s.d. (c) Specific cytolytic activity of CD8⁺ FLT3 CAR-T cells and CD8⁺ CD123 CAR-T cells against primary AML blasts analyzed in a 4-hour flow cytometry-based cytotoxicity assay. Assay was performed in triplicate wells at the indicated effector to target cell ratio with 10,000 target cells/well. Counting beads were used to quantitate the number of residual live primary AML blasts at the end of the co-culture and calculate specific lysis.

FIG. 5: FLT3 CAR-T cells produce effector cytokines and proliferate after stimulation with MOLM-13 AML cells. (a) Enzyme linked immune sorbent assay (ELISA) to detect IFN-γ and IL-2 in supernatant obtained from 24-hour co-cultures of CD4⁺ and CD8⁺ FLT3 CAR-T cells with MOLM-13 target cells at 2:1 E:T ratio. Values are presented as mean±s.d. (b) Proliferation of FLT3 CAR-T cells examined by carboxyfluorescein succinimidyl ester (CFSE) dye dilution after 72 hours of co-culture with MOLM-13 target cells at 2:1 E:T ratio. Histograms show proliferation of live (7-AAD⁻) CD4⁺ or CD8⁺ T cells. No exogenous cytokines were added to the assay medium. Data shown are representative for results obtained with FLT3 CAR-modified and control T-cell lines prepared from at least n=5 donors.

FIG. 6: FLT3 CAR-T cells produce effector cytokines and proliferate after stimulation with THP-1 AML cells. (a) Enzyme linked immune sorbent assay (ELISA) to detect IFN-γ and IL-2 in supernatant obtained from 24-hour co-cultures of CD4⁺ and CD8⁺ FLT3 CAR-T cells with MOLM-13 target cells at 2:1 E:T ratio. Values are presented as mean±s.d. (b) Proliferation of FLT3 CAR-T cells examined by carboxyfluorescein succinimidyl ester (CFSE) dye dilution after 72 hours of co-culture with MOLM-13 target cells at 2:1 E:T ratio. Histograms show proliferation of live (7-AAD⁻) CD4⁺ or CD8⁺ T cells. No exogenous cytokines were added to the assay medium. Data shown are representative for results obtained with FLT3 CAR-modified and control T-cell lines prepared from at least n=5 donors.

FIG. 7: FLT3 CAR-T cells confer potent antileukemia activity in a xenograft model of AML in immunodeficient mice in vivo. Six-8 week old female NSG mice were inoculated with 1×10⁶ MOLM-13 AML cells [firefly luciferase (ffluc)⁺/green fluoresence protein (GFP)⁺] and treated with 5×10⁶ CAR-modified or UTD T cells on day 7, or were left untreated. (a) Serial bioluminesence imaging (BLI) to assess leukemia progression and regression in each treatment group. Note the scale (right) indicating upper and lower BL thresholds at each analysis time point. (b) Flow cytometric anaysis of peripheral blood on day 3 after T-cell transfer (i.e. day 10 after leukemia inoculation). Data show the frequency of transferred T cells (CD45⁺/CD3⁺) in each of the treatment groups as percentage of live (7-AAD⁻) cells.

FIG. 8: FLT3 CAR-T cells confer potent antileukemia activity in a xenograft model of AML in immunodeficient mice in vivo. (a) Flow cytometric anaysis of peripheral blood (PB), spleen (Sp) and bone marrow (BM) at the experimental endpoint in each mouse. Dot plots show the frequency of leukemia cells (GFP⁺/FLT3⁺) as percentage of live (7-AAD⁻) cells in one representative mouse per group. Diagrams show the frequency of leukemia cells (GFP⁺/FLT3⁺) as percentage of live (7-AAD⁻) cells. p<0.05 (Student's t-test). (b) Waterfall plot showing the A (increase/decrease) in absolute bioluminesence values obtained from each of the mice between day 7 and day 14 of the experiment [i.e. (day 14)−(day 7) after tumor inoculation, i.e. (day 7 after)−(before) T-cell transfer]. Bioluminesence values were obtained as photon/sec/cm²/sr in regions of interest encompassing the entire body of each mouse.

FIG. 9: FLT3 CAR-T cells show long-term persistance after adoptive transfer and lead to improved survival of NSG/MOLM-13 mice. (a) Flow cytometric dot plots from bone marrow, spleen and peripheral blood of a representative mouse from each treatment group. Diagram in right represents percentage of CD8⁺ T cells in UTD or FLT3 CAR T cells treated mice. Values are presented as mean±s.d. (b) Kaplan-Meier analysis of survival in each of the treatment groups. As per protocol, experimental endpoints were defined by relative (%) loss of body weight and total bioluminescence values. p<0.05 (Log-rank test). Data shown are representative for results obtained in independent experiments with FLT3 CAR-T cells lines prepared from n=3 donors.

FIG. 10: Midostaurin treatment leads to enhanced FLT3 expression on AML cells. (a) Flow cytometric analysis of FLT3 expression on MOLM-13, MV4;11, THP-1, K562 cells that had been cultured in the presence of 10 nM midostaurin for 15 days follwed by serial increment upto 50 nM concentration by the end of 3 months. Histograms show staining with anti-FLT3 mAb (4G8) (gray histograms) compared to isotype (black histograms). ΔMFI (Difference in mean fluoresence intensity) values represents absolute difference in MFI of non-treated and 50 nM midostaurin treated cells [i.e. (MFI of 50 nM midostaurin treated)−(MFI of non-treated)]. (b) Flow histograms show FLT3 expression on MOLM-13 cells that had been cultured in the presence of 10 nM midostaurin for 2-3 weeks followed by serial increment upto 50 nM concentration by in next 8-10 weeks. (c) Flow histograms show FLT3 expression on MOLM-13 cells after exposure to 50 nM midostaurin (exposure), 2 days after subsequently withdrawing the drug (withdrawal), and 7 days afer re-exposure to 50 nM crenolanib (re-exposure).

FIG. 11: MOLM-13^{mido} showed lower CD33 and CD123 expression in vitro. (a) Flow cytometric analysis of CD33 and CD123 expression on MOLM-13^{native} (dark grey) and MOLM-13^{mido} (light grey) cells. Representative data from n=2 independent experiments.

FIG. 12: FLT3 CAR-T cells exert enhanced cytotoxicity against MOLM-13^{mido} in vitro. (a) Recognition of MOLM-13^{mido} and MOLM-13^{native} AML cells by FLT3 CAR-T cells. Assays with MOLM-13^{mido} were performed in medium containing 50 nM midostaurin. Cytolytic activity in a bioluminescence-based cytotoxicity assay (4-hour incubation at a 10:1 E:T ratio with 5,000 target cells/well). Data shown are representative for results obtained in independent experiments with FLT3 CAR-T cells lines prepared from n=2 donors. **p<0.005, ***p<0.0005 (Student's t-test).

FIG. 13: FLT3 CAR-T cells show enhanced cytokine production and proliferation against MOLM-13^{mido} in vitro. (a) IFN-γ and IL-2 ELISA (24-hour incubation at a 4:1 E:T ratio with 50,000 T cells/well). (b) Proliferation of CD4⁺ FLT3 CAR-T cells assessed by CFSE dye dillution (72-hour co-culture of 50,000 T cells with 12,500 target cells/well). Data shown are representative for results obtained in independent experiments with FLT3 CAR-T cells lines prepared from n=2 donors. ****p<0.0001 (Student's t-test).

FIG. 14: Crenolanib treatment leads to enhanced FLT3 expression on AML cells. (a) Flow cytometric analysis of FLT3 expression on MOLM-13, MV4;11, THP-1, K562 cells that had been cultured in the presence of 10 nM crenolanib for 7 days, compared to non-treated cells. Histograms show staining with anti-FLT3 mAb (4G8) (gray histograms) compared to isotype (black histograms). ΔMFI (Difference in mean fluoresence intensity) values represents absolute difference in MFI of non-treated and 10 nM crenolanib treated cells [i.e. (MFI of 10 nM crenolanib treated)−(MFI of non-treated)]. (b) Flow histograms show FLT3 expression on MOLM-13 cells 7 days after exposure to 10 nM crenolanib (exposure), 2 days after subsequently withdrawing the drug (withdrawal), and 7 days afer re-exposure to 10 nM crenolanib (re-exposure).

FIG. 15: Crenolanib treatment leads to enhanced FLT3 expression on MOLM-13. efluro 670 dye labelled 1×10⁶ MOLM-13 cells were plated in 48 well plate (in triplicate wells) on day 0 in 1 mL culture medium with or without 10 nM crenolanib. (a) After 5 and 10 days, cells were washed and stained for FLT3 expression using anti-FLT3 mAb. efluro 647 dye labelling was used to track proliferation. Solid line denotes untreated (0 nM) and zebra line denotes 10 nM crenolanib treated MOLM-13 cells. Representative data from n=2 independent experiments. (b) Percentage of MOLM-13 dead cells (7-AAD⁺ cells) after 0 nM and 10 nM crenolanib treatment. Black arrows denote medium change with fresh drug supplement. Data represents mean+s.d. from n=2 independent experiments.

FIG. 16: CD33 and CD123 expression is not altered on MOLM-13^{creno}. (a) Flow cytometric analysis of CD33 and CD123 expression on MOLM-13^{native} (dark grey) and MOLM-43^{creno} (light grey) cells. Representative data from n=2 independent experiments.

FIG. 17: FLT3 CAR-T cells exert enhanced cytotoxicity against MOLM-13^{creno} in vitro. (a) Recognition of MOLM-13^{creno} and MOLM-13^{native} AML cells by FLT3 CAR-T cells. Assays with MOLM-13^{creno} were performed in medium containing 10 nM crenolanib. Cytolytic activity in a bioluminescence-based cytotoxicity assay (4-hour incubation at a 10:1 E:T ratio with 5,000 target cells/well). Data shown are representative for results obtained in independent experiments with FLT3 CAR-T cells lines prepared from n=2 donors. *p<0.05, **p<0.005 (Student's t-test).

FIG. 18: FLT3 CAR-T cells show enhanced cytokine production and proliferation against MOLM-13^{creno} in vitro. (a) IFN-γ and IL-2 ELISA (24-hour incubation at a 4:1 E:T ratio with 50,000 T cells/well). (b) Proliferation of CD4⁺ FLT3 CAR-T cells assessed by CFSE dye dillution (72-hour co-culture of 50,000 T cells with 12,500 target cells/well). Data shown are representative for results obtained in independent experiments with FLT3 CAR-T cells lines prepared from n=2 donors. *p<0.05, ***p<0.0005 (Student's t-test).

FIG. 19: Quizartinib treatment leads to enhanced FLT3 expression on AML cells. (a) Flow cytometric analysis of FLT3 expression on MOLM-13, MV4;11, THP-1, K562 cells that had been cultured in the presence of 1 nM quizartinib for 7 days, compared to non-treated cells. Histograms show staining with anti-FLT3 mAb (4G8) (gray histograms) compared to isotype (black histograms). ΔMFI (Difference in mean fluoresence intensity) values represents absolute difference in MFI of non-treated and 1 nM quizartinib treated cells [i.e. (MFI of 1 nM quizartinib treated)−(MFI of non-treated)]. (b) Flow histograms show FLT3 expression on MOLM-13 cells 7 days after exposure to 1 nM quizartinib (exposure), 2 days after subsequently withdrawing the drug (withdrawal), and 7 days afer re-exposure to 1 nM quizartinib (re-exposure).

FIG. 20: CD33 and CD123 expression is not altered on MOLM-13^{quiza}. (a) Flow cytometric analysis of CD33 and CD123 expression on MOLM-13^{native} (dark grey) and MOLM-13^{quiza} (light grey) cells. Representative data from n=2 independent experiments.

FIG. 21: FLT3 CAR-T cells show enhanced cytotoxicity against MOLM-13^{quiza} in vitro. (a) Recognition of MOLM-13^{quiza} and MOLM-13^{native} AML cells by FLT3 CAR-T cells. Assays with MOLM-13^{quiza} were performed in medium containing 1 nM quizartinib. Cytolytic activity in a bioluminescence-based cytotoxicity assay (4-hour incubation at a 10:1 E:T ratio with 5,000 target cells/well). Data shown are representative for results obtained in independent experiments with FLT3 CAR-T cells lines prepared from n=2 donors. *p<0.05, **p<0.005 (Student's t-test).

FIG. 22: FLT3 CAR-T cells show enhanced cytokine production and proliferation against MOLM-13^{quiza} in vitro. (a) IFN-γ and IL-2 ELISA (24-hour incubation at a 4:1 E:T ratio with 50,000 T cells/well). (b) Proliferation of CD4⁺ FLT3 CAR-T cells assessed by CFSE dye dillution (72-hour co-culture of 50,000 T cells with 12,500 target cells/well). Data shown are representative for results obtained in independent experiments with FLT3 CAR-T cells lines prepared from n=2 donors. **p<0.005, ***p<0.0005 (Student's t-test).

FIG. 23: Crenolanib acts synergistically with FLT3 CAR-T cells and enhances antileukemic efficacy of FLT3 CAR-T cells in vivo. Six-8 weeks old female NSG mice were inoculated with 1×10⁶ MOLM-13 cells (ffluc⁺GFP⁺) and treated with 5×10⁶ FLT3 CAR T cells alone, crenolanib alone (15 mg/kg body weight as i.p. injection) or both on day 7 or were left untreated. First dose of crenolanib was given on day 7 and mice received 15 doses for 3 consecutive weeks (Monday-Friday). (a) Serial bioluminesence imaging to assess leukemia progression and regression in each treatment group. Note the scale (right) indicating upper and lower BL thresholds at each analysis time point. (b) Percentage of live (7-AAD⁻) T cells (CD45⁺ CD3⁺) in peripheral blood (on day 4 after T cells injection, i.e. after 5 doses of crenolanib) of mice which received FLT3 CAR T cells only or crenolanib with FLT3 CAR T cells (upper diagram). Mice from untreated and cenolanib only treated group were analyzed (after 5 doses of crenolanib) for FLT3 expression on live (7-AAD⁻) leukemic cells (GFP⁺ CD45⁺) from bone marrow (lower diagram). Data were analyzed using students t-test (*p<0.05, **p<0.005)

FIG. 24: Crenolanib acts synergistically with FLT3 CAR-T cells and enhances antileukemic efficacy of FLT3 CAR-T cells in vivo. (a) Water fall plot showing the difference in absolute bioluminesence values obtained from each of the mice between day 7 and day 14 after tumor inoculation. [i.e. (day 14)−(day 7) after tumor inoculation, i.e. (day 7 after)−(before) T-cell transfer]. Bioluminesence values were obtained as photon/sec/cm²/sr in regions of interest encompassing the entire body of each mouse. (b) Kaplan-Meier analysis of survival in each of the treatment group. As per protocol, experimental endpoints were defined by relative (%) loss of body weight and total bioluminescence values. *p<0.05 (Log-rank test).

FIG. 25: Combination treatment of Crenolanib with FLT3 CAR-T cells leads to significantly enhanced survival of NSG/MOLM-13 mice compared to monotherapy. (a) Expression of FLT3 was analyzed on MOLM-13 cells obtained from peripheral blood of mice that had either been treated with crenolanib or not. *p<0.05 (Student's t-test). (b) Diagrams show the frequency of leukemia cells (GFP⁺/CD45⁺) as percentage of live (7-AAD⁻) cells obtained from bone marrow, spleen and peripheral blood. *p<0.05, **p<0.005 (Student's t-test). Data shown are representative for results obtained in independent experiments with FLT3 CAR-T cells lines prepared from n=2 donors.

FIG. 26: Phenotype of CAR T cells after EGFRt enrichment

T cells isolated from healthy donor or AML patients peripheral blood mononuclear cells were stimulated with CD3/CD28 beads, CAR transgene was lentivirally transduced, stained (after 8-10 days) with biotinylated anti-tEGFR antibody followed by anti-biotin magnetic beads staining and sorted using Magnetic-Activated Cell Sorting (MACS). Flow cytometric analysis of CAR expression by CD8⁺ and CD4⁺ T cells after MACS sorting.

FIG. 27: FLT3 CAR-T cells specifically recognized FLT3⁺ K562 tumor cells

K562/FLT3 was generated by retroviral transduction with the full-length human FLT3 gene. (a) Flow cytometric analysis of FLT3 expression by K562 native and K562/FLT3 cells. (b) Specific cytolytic activity of CD8⁺ FLT3 CAR-T cells, analyzed after 4-hour in a bioluminescence-based cytotoxicity assay. Values are presented as mean+s.d. The right-hand graph shows cytolytic activity of CAR T cells prepared from three different T cell donors.

FIG. 28: FLT3 CAR-T cells recognize and eliminate FLT3 wild-type and FLT3-ITD⁺ AML cell lines and primary AML cells in vitro. (a) Flow cytometric analysis of FLT3 expression on AML cell lines (MOLM-13, THP-1, MV4;11) and primary AML blasts (pt #1 and #2). Histograms show staining with anti-FLT3 mAb (4G8) (solid line) and isotype control antibody (zebra line). ΔMFI (Difference in mean fluoresence intensity) values represents absolute difference in MFI of anti-FLT3 mAb stained and isotype control stained cells. (b) Specific cytolytic activity of CD8⁺ FLT3 CAR-T cells, CD19 CAR-T cells or untransduced T cells (UTD) against AML cell lines analyzed after 4-hour in a bioluminescence-based cytotoxicity assay. Assay was performed in triplicate wells at the indicated effector to target cell ratio with 5,000 target cells/well. Values are presented as mean+s.d. (c) Specific cytolytic activity of CD8⁺ FLT3 CAR-T cells and CD8⁺ CD123 CAR-T cells against primary AML blasts analyzed in a 4-hour flow cytometry-based cytotoxicity assay. Assay was performed in triplicate wells at the indicated effector to target cell ratio with 10,000 target cells/well. Counting beads were used to quantitate the number of residual live primary AML blasts at the end of the co-culture and calculate specific lysis.

FIG. 29: FLT3 CAR-T cells produce effector cytokines and proliferate against MOLM-13 AML cells.

(a) Enzyme linked immune sorbent assay (ELISA) to detect IFN-γ and IL-2 in supernatant obtained from 24-hour co-cultures of CD4⁺ and CD8⁺ FLT3 CAR-T cells with MOLM-13 target cells at 2:1 E:T ratio. Values are presented as mean±s.d. (b) Proliferation of FLT3 CAR-T cells examined by carboxyfluorescein succinimidyl ester (CFSE) dye dilution after 72 hours of co-culture with MOLM-13 target cells at 2:1 E:T ratio. Histograms show proliferation of live (7-AAD⁻) CD4⁺ or CD8⁺ T cells. No exogenous cytokines were added to the assay medium. Data shown are representative for results obtained with FLT3 CAR-modified and control T-cell lines prepared from at least n=5 donors.

FIG. 30: FLT3 CAR-T cells produce effector cytokines and proliferate against THP-1 AML cells.

(a) Enzyme linked immune sorbent assay (ELISA) to detect IFN-γ and IL-2 in supernatant obtained from 24-hour co-cultures of CD4⁺ and CD8⁺ FLT3 CAR-T cells with MOLM-13 target cells at 2:1 E:T ratio. Values are presented as mean±s.d. (b) Proliferation of FLT3 CAR-T cells examined by carboxyfluorescein succinimidyl ester (CFSE) dye dilution after 72 hours of co-culture with MOLM-13 target cells at 2:1 E:T ratio. Histograms show proliferation of live (7-AAD⁻) CD4⁺ or CD8⁺ T cells. No exogenous cytokines were added to the assay medium. Data shown are representative for results obtained with FLT3 CAR-modified and control T-cell lines prepared from at least n=5 donors.

FIG. 31: FLT3 CAR-T cells confer potent antileukemia activity in a xenograft model of AML in immunodeficient mice in vivo. Six-8 week old female NSG mice were inoculated with 1×10⁶ MOLM-13 AML cells [firefly luciferase (ffluc)⁺/green fluoresence protein (GFP)⁺] and treated with 5×10⁶ CAR-modified or UTD T cells on day 7, or were left untreated. (a) Serial bioluminesence imaging (BLI) to assess leukemia progression and regression in each treatment group. Note the scale (right) indicating upper and lower BL thresholds at each analysis time point. (b) Flow cytometric anaysis of peripheral blood on day 3 after T-cell transfer (i.e. day 10 after leukemia inoculation). Data show the frequency of transferred T cells (CD45⁺/CD3⁺) in each of the treatment groups as percentage of live (7-AAD⁻) cells.

FIG. 32: FLT3 CAR-T cells reduce leukemia burden and improve survival in a xenograft model of AML in immunodeficient mice in vivo.

(a) Waterfall plot showing the A (increase/decrease) in absolute bioluminesence values obtained from each of the mice between day 7 and day 14 of the experiment [i.e. (day 14)−(day 7) after tumor inoculation, i.e. (day 7 after)−(before) T-cell transfer]. Bioluminesence values were obtained as photon/sec/cm²/sr in regions of interest encompassing the entire body of each mouse. (b) Kaplan-Meier analysis of survival in each of the treatment groups. As per protocol, experimental endpoints were defined by relative (%) loss of body weight and total bioluminescence values. p<0.05 (Log-rank test). Data shown are representative for results obtained in independent experiments with FLT3 CAR-T cells lines prepared from n=3 donors.

FIG. 33: FLT3 CAR-T cells eliminate AML from bone marrow, spleen and peripheral blood in vivo

(a) Flow cytometric analysis from bone marrow, spleen and peripheral blood of a representative mouse from each treatment group. Values are presented as mean±s.d.

FIG. 34: FLT3 CAR-T cells exert enhanced cytotoxicity against MOLM-13^{mido} in vitro.

(a) Recognition of MOLM-13^{mido} and MOLM-13^{native} AML cells by FLT3 CAR-T cells. Assays with MOLM-13^{mido} were performed in medium containing 50 nM midostaurin. Cytolytic activity in a bioluminescence-based cytotoxicity assay (4-hour incubation at different E:T ratio with 5,000 target cells/well). Data shown are representative for results obtained in independent experiments with FLT3 CAR-T cells lines prepared from n=2 donors. *p<0.05, **p<0.005 (Student's t-test).

FIG. 35: FLT3 CAR-T cells show enhanced cytokine production and proliferation against MOLM-13^{mido} in vitro.

(a) IFN-γ and IL-2 ELISA (24-hour incubation at a 4:1 E:T ratio with 50,000 T cells/well). (b) Proliferation of CD4⁺ FLT3 CAR-T cells assessed by CFSE dye dillution (72-hour co-culture of 50,000 T cells with 12,500 target cells/well). Data shown are representative for results obtained in independent experiments with FLT3 CAR-T cells lines prepared from n=2 donors. ***p<0.0005 (Student's t-test).

FIG. 36: FLT3 CAR-T cells exert enhanced cytotoxicity against MOLM-13^{creno} in vitro.

(a) Recognition of MOLM-13′ and MOLM-13^{native} AML cells by FLT3 CAR-T cells. Assays with MOLM-13^{creno} were performed in medium containing 10 nM midostaurin. Cytolytic activity in a bioluminescence-based cytotoxicity assay (4-hour incubation at a 10:1 E:T ratio with 5,000 target cells/well). Data shown are representative for results obtained in independent experiments with FLT3 CAR-T cells lines prepared from n=2 donors. *p<0.05, **p<0.005 (Student's t-test).

FIG. 37: FLT3 CAR-T cells show enhanced cytokine production and proliferation against MOLM-13^{creno} in vitro.

(a) IFN-γ and IL-2 ELISA (24-hour incubation at a 4:1 E:T ratio with 50,000 T cells/well). (b) Proliferation of CD4⁺ FLT3 CAR-T cells assessed by CFSE dye dillution (72-hour co-culture of 50,000 T cells with 12,500 target cells/well). Data shown are representative for results obtained in independent experiments with FLT3 CAR-T cells lines prepared from n=2 donors. *p<0.05, **p<0.005 (Student's t-test).

FIG. 38: FLT3 CAR-T cells exert enhanced cytotoxicity against MOLM-13^{quiza} in vitro.

(a) Recognition of MOLM-13^{quiza} and MOLM-13^{native} AML cells by FLT3 CAR-T cells. Assays with MOLM-13^{quiza} were performed in medium containing 1 nM midostaurin. Cytolytic activity in a bioluminescence-based cytotoxicity assay (4-hour incubation at a 10:1 E:T ratio with 5,000 target cells/well). Data shown are representative for results obtained in independent experiments with FLT3 CAR-T cells lines prepared from n=2 donors. *p<0.05, **p<0.005 (Student's t-test).

FIG. 39: FLT3 CAR-T cells show enhanced cytokine production and proliferation against MOLM-13^{quiza} in vitro.

(a) IFN-γ and IL-2 ELISA (24-hour incubation at a 4:1 E:T ratio with 50,000 T cells/well). (b) Proliferation of CD4⁺ FLT3 CAR-T cells assessed by CFSE dye dillution (72-hour co-culture of 50,000 T cells with 12,500 target cells/well). Data shown are representative for results obtained in independent experiments with FLT3 CAR-T cells lines prepared from n=2 donors. **p<0.005, ***p<0.0005 (Student's t-test).

FIG. 40: Midostaurin acts synergistically with FLT3 CAR-T cells and enhances anti-leukemia activity of FLT3 CAR-T cells in vivo. 6-8 week old female NSG immunodeficient mice were injected with 1×10⁶ ffluc+GFP+ MOLM-13 cells on day 0. On day 7, mice were treated with a single dose of FLT3 CAR-T cells alone (5×10⁶ cells, CD4+:CD8+ ratio=1:1), midostaurin alone (1 mg/kg body weight as i.p. injection), or both (combination), or were left untreated. Mice in the FLT3 CAR+early mido group received midostaurin on day 3, 4, 5 and received additional 12 doses of midostaurin starting from day 7. Mice in the FLT3 CAR+midostaurin group received the first dose of midostaurin on day 7 (i.e. the same day of T cell injection) and received total 15 doses of midostauin for 3 consecutive weeks (Monday-Friday). (a) Serial bioluminescence (BL) imaging to assess leukemia progression/regression in each treatment group. Note the scale (right) indicating upper and lower BL thresholds at each analysis time point. (b) Water fall plot representing the fold change in BL value between day 7 and day 11 after tumor inoculation. BL values were obtained as photon/sec/cm²/sr.

FIG. 41: FLT3 CAR-T cell expansion and FLT3 expression on MOLM-13 cells after midostaurin treatment in vivo. (a) Peripheral blood analysis (on day 11 after tumor inoculation) of mice treated with FLT3 CAR-T cells alone or in combination with midostaurin. Diagram shows percentage of live (7-AAD−) T-cells (CD45+CD3+) in peripheral blood. *p<0.05, **p<0.005 (Student's t-test). (b) Flow cytometric analysis of FLT3-expression on MOLM-13 cells was performed on the cells obtained from bone marrow of untreated and midostaurin treated mice (after 5 doses of midostaurin). Diagram shows mean fluorescence intensity (MFI) of FLT3.

FIG. 42: Quizartinib acts synergistically with FLT3 CAR-T cells and enhances anti-leukemia activity of FLT3 CAR-T cells in vivo. Female NSG immunodeficient mice (6-8 week old) were inoculated with 1×10⁶ ffluc+GFP+ MOLM-13 cells on day 0. On day 7, mice were treated with a single dose of FLT3 CAR-T cells alone (5×10⁶ cells, CD4+:CD8+ ratio=1:1), quizartinib alone (1 mg/kg body weight as i.p. injection), or both (combination), or were left untreated. Mice in the FLT3 CAR+ quizartinib group received the first dose of quizartinib on day 7 (i.e. the same day of T cell injection) and mice received a total of 15 doses of quizartinib for 3 consecutive weeks (Monday-Friday). (a) Serial bioluminescence (BL) imaging to assess leukemia progression/regression in each treatment group. (b) Water fall plot represents the fold change in BL value between day 7 and day 10 after tumor inoculation. BL values were obtained as photon/sec/cm²/sr.

FIG. 43: FLT3 CAR-T cells expansion and analysis of FLT3 expression on MOLM-13 cells after quizartinib treatment in vivo. (a) Peripheral blood analysis (on day 10 after tumor inoculation) of mice treated with FLT3 CAR-T cells alone or in combination with quizartinib. Diagram shows the percentage of live (7-AAD−) T-cells (CD45+CD3+) in peripheral blood. **p<0.005 (Student's t-test). (b) Flow cytometric analysis of FLT3-expression on MOLM-13 cells was performed on the cells obtained from bone marrow of untreated and quizartinib treated mice (after 5 doses of quizartinib). Diagram shows mean fluorescence intensity (MFI) of FLT3.

FIG. 44: FLT3 expression on acute lymphoblastic leukemia (ALL) and mixed-lineage leukemia (MLL) cell lines and their recognition by FLT3 CAR-T cells in vitro. (a) Flow cytometric analysis of FLT3 expression on ALL (NALM-16) cells and MLL (KOPN-8 and SEM) cells. Inset number represents absolute difference between MFI of anti-FLT3 and isotype staining. (b) Specific cytolytic activity in 4-hour cytotoxicity assay with FLT3 CAR-T cells vs ALL and MLL cell lines as target cells. Values represent mean±s.d.

FIG. 45: IL-2 production and proliferation mediated by CD4+FLT3 CAR-T cells against ALL and MLL cell lines. (a) IL-2 production by FLT3 CAR-T cells measured by ELISA after a 24-hour incubation with target cells at a 2:1 E:T ratio (50,000 T-cells/well). (b) Proliferation of FLT3 CAR-T and control CD19 CAR-T cells examined by CFSE dye dilution after 72 hour of co-culture with target cells. Representative data of T cells prepared from n=2 different donors.

FIG. 46: FLT3 expression on ALL and MLL cell lines after treatment with FLT3 inhibitors. (a) Flow cytometry analysis of FLT3-expression on ALL and MLL cell lines which were cultured in the absence or presence of 50 nM midostaurin, 10 nM crenolanib or 1 nM quizartinib for 1 week.

FIG. 47: Antibody dependent cellular cytotoxicity (ADCC) against MV4;11 AML cells with and without FLT3 inhibitors pretreatment. MV4;11 AML cells were pretreated with FLT3 inhibitors (10 nM crenolanib, 1 nM quizartinib or 50 nM midostaurin) for 7 days. Healthy donor derived PBMCs (effector/target ratio of 50:1) and control IgG1 antibody or anti-FLT3 BV10 mAb were added at a concentration of 5000 ng/mL. MV4;11 cells stably expressed firefly luciferase, and cell viability was analyzed after the addition of luciferin substrate by bioluminescence measurements after 24 hours of co-culture. Values are presented as mean±SD. P values between indicated groups were calculated by using an unpaired Student's t test. *P<0.05; **P<0.005.

### DETAILED DESCRIPTION OF THE INVENTION

The invention generally relates to the treatment of cancer with FLT3 targeting agents and kinase inhibitors. In particular, the invention relates to the treatment of Acute Myeloid Leukemia (AML) with T cells that were modified by gene-transfer to express an FLT3-specific chimeric antigen receptor (CAR) in combination with FLT3 inhibitors. In the present invention, the inventors demonstrate that treatment of AML blasts with FLT3 inhibitors leads to a significant increase in expression of the FLT3 molecule on the cell surface of AML blasts, which as a consequence leads to a significant increasing in recognition and elimination by FLT3 CAR-T cells. The combination treatment of AML with FLT3 CAR-T cells and FLT3 inhibitors is highly synergistic and superior to monotherapy with either FLT3 inhibitors or FLT3 CAR-T cells alone.

Recent clinical trials have demonstrated that adoptive immunotherapy with CD19 CAR-T cells in B-lineage leukemia and lymphoma; as well as with BCMA (B-cell maturation antigen) CAR-T cells in multiple myeloma can be effective against advanced hematologic malignancies. However, these clinical trials have also demonstrated that there is a substantial risk of relapse due to emergence of antigen-loss tumor variants, as recently demonstrated on example of CD19 CARs (leukemia relapse due to emergence of CD19-negative leukemia variants, Ref.: Turtle et al J Clin Invest 2016, PMID: 27111235) and BCMA CARs (myeloma relapse due to emergence of BCMA-negative/low myeloma variants, Ref.: Ali et al. Blood 2016, PMID: 27412889). There are several explanations why antigen-loss occurs after CAR-T cell therapy, including that i) the CAR target antigen is not uniformly expressed or not expressed at high enough levels; ii) the CAR target antigen is not of pathophysiologic relevance for the tumor such that loss of the antigen can be tolerated by the tumor cells. Thus far, no methods have been described to prevent the occurrence of antigen loss tumor variants when under therapeutic pressure from CAR-T cells. The inventors reason however, that CAR-T cell therapy would be more effective and have a higher chance to cure the underlying hematologic malignancy in a greater percentage of patients if there were means that force tumor cells to augment expression of the CAR target antigen expression on their cell surface and prevent tumor cells from losing the antigen. The inventors demonstrate in this invention that it is possible to force AML blasts to augment expression of the FLT3 molecule through treatment with FLT3 inhibitors. As a consequence, recognition and elimination of AML blasts by FLT3 CAR-T cells is significantly enhanced in vitro and in vivo. Because treatment of AML blasts with FLT3 inhibitors leads to enhanced expression of the FLT3 molecule on all AML blasts, the chance to eliminate all AML blasts with FLT3 CAR-T cells is higher and the chance that AML blasts escape elimination by FLT3 CAR-T cells is lower. Hence, there is a higher chance to cure AML through combination treatment with FLT3 CAR-T cells and FLT3 inhibitors compared to treatment with FLT3 inhibitors alone or FLT3 CAR-T cells alone.

FLT3 inhibitors are being used to treat AML however, as single agents there clinical efficacy is low and they are not able to cure the disease in the overwhelming majority of patient. The consequences of targeting AML blasts with FLT3 inhibitors on the expression of the FLT3 molecule in AML blasts are unpredictable: i) it may be that expression of FLT3 is lowered because of the direct toxic effect of FLT3 inhibitors which perturbates protein synthesis and turnover; ii) it may be that expression of FLT3 is unchanged because AML blasts commonly acquire novel mutations in the FLT3 molecule that render FLT3 inhibitors ineffective, or switch to and use alternative molecular survival pathways; iii) it may also be that expression of FLT3 on AML blasts is increased to compensate inhibition conferred by the FLT3 inhibitor.

The inventors show that treatment of AML blasts with the FLT3 inhibitors midostaurin, quizartinib and crenolanib leads to a significant increase in FLT3 expression, particularly in AML blasts that carry the FLT3 internal tandem duplication (FLT3-ITD). The increase in FLT3 expression on AML blasts occurs rapidly after the onset of FLT3 inhibitor treatment and leads to significantly enhanced recognition by FLT3 CAR-T cells (stronger and more rapid cytolytic activity; stronger cytokine secretion including IL-2; stronger and more rapid proliferation; superior viability and survival after stimulation with AML blasts). Further, combination treatment of AML with FLT3 CAR-T cells and FLT3 inhibitors lead to significantly enhanced CAR-T cell persistence and antileukemia function in a mouse model of AML in vivo. The increase in FLT3 expression on AML blasts can be modulated and rapidly returns to baseline levels if treatment with FLT3 inhibitor is terminated. Surprisingly, the viability and function of FLT3 CAR-T cells was not affected by midostaurin, quizartinib and crenolanib even though each of the substances is a multi-kinase inhibitor and may therefore interfere with signaling and function of the FLT3 CAR.

### Definitions and Embodiments

Unless otherwise defined below, the terms used in the present invention shall be understood in accordance with the common meaning known to the person skilled in the art.

Each publication, patent application, patent, and other reference cited herein is incorporated by reference in its entirety to the extent that it is not inconsistent with the present invention. References are indicated by their reference numbers and their corresponding reference details which are provided in the “references” section.

A “kinase inhibitor” as referred to herein is a molecular compound which inhibits one or more kinase(s) by binding to said kinase(s) and exerting an antagonistic effect on said kinase. A kinase inhibitor is capable of binding to one or more kinase species, upon which the kinase activity of the one or more kinase is reduced. A kinase inhibitor as described herein is typically a small molecule, wherein a small molecule is a molecular compound of low molecular weight (typically less than 1 kDa) and size (typically smaller than 1 nM).

In one embodiment, the kinase inhibitor is a multikinase inhibitor. As used herein, a “multikinase inhibitor” is a kinase inhibitor capable of inhibiting more than one type of kinase. In a preferred embodiment, the kinase inhibitor is a tyrosine kinase inhibitor. In another preferred embodiment, the kinase inhibitor is an FLT3 inhibitor. In a more preferred embodiment, the kinase inhibitor inhibits mutated FLT3, more preferably FLT3-ITD. In a more preferred embodiment, the kinase inhibitor is an FLT3 kinase inhibitor selected from the group consisting of crenolanib, midostaurin, and quizartinib. In a very preferred embodiment, the kinase inhibitor is the FLT3 kinase inhibitor crenolanib.

As used herein, “type II receptor tyrosine kinase inhibitors” target an inactive conformation of the receptor tyrosine kinase, whereas “type I receptor tyrosine kinase inhibitors” target an active conformation of the receptor tyrosine kinase. An exemplary type II receptor tyrosine kinase inhibitor is the FLT3 inhibitor quizartinib. An exemplary type I receptor tyrosine kinase inhibitor is the FLT3 inhibitor crenolanib.

The terms “K_{D}” or “K_{D} value” relate to the equilibrium dissociation constant as known in the art. In the context of the present invention, these terms relate to the equilibrium dissociation constant of a targeting agent with respect to a particular antigen of interest (e.g. FLT3). The equilibrium dissociation constant is a measure of the propensity of a complex (e.g. an antigen-targeting agent complex) to reversibly dissociate into its components (e.g. the antigen and the targeting agent). Methods to determine K_{D} values are known in art.

A targeting agent as described herein is an agent that, contrary to common medical agents, is capable of binding specifically to its target.

The targeting agent according to the invention is an FLT3 targeting agent. A preferred targeting agent in accordance with the invention is capable of binding to FLT3 on the cell surface, typically to the extracellular domain of the transmembrane protein FLT3.

In one embodiment of the invention, the targeting agent is capable of binding specifically to tumor cells expressing FLT3. In another embodiment of the invention, the targeting agent is capable of binding specifically to hematopoietic cells expressing FLT3. In another embodiment of the invention, the targeting agent is capable of binding specifically to hematopoietic tumor cells expressing FLT3. In a preferred embodiment of the invention, the targeting agent is capable of binding to acute myeloid leukemia cells expressing FLT3. In a very preferred embodiment of the invention, the targeting agent is capable of binding to acute myeloid leukemia cells which express mutated FLT3, preferably FLT3-ITD.

Terms such as “growth inhibition of cells” as used herein mean the effect of causing a decrease in cell number. Preferably, this can be caused by cytotoxicity through necrosis or apopotisis, or this can be caused by inhibiting or stopping proliferation. A “growth inhibiting effect” as used herein means that a substance, molecule, compound, composition or agent has a growth inhibiting effect on the cells as compared to a situation where said substance, molecule, compound, composition, or agent is not present. Cell growth inhibition can be measured by various common methods and assays known in the art.

Whenever the present invention refers to a composition, a composition for use, a kit, a use, a method, a combination, a combination for use and the like which relates to (a) a kinase inhibitor; and (b) an FLT3-targeting agent, it is to be understood that the kinase inhibitor is different from the FLT3-targeting agent.

Further, it is also to be understood that terms such as “a kinase inhibitor” refer to the presence of a kinase inhibitor but do not exclude the possibility that additional kinase inhibitors, e.g. one, two, three or more additional kinase inhibitors could be present. In one embodiment in accordance with the invention, only one kinase inhibitor is used.

It is also to be understood that terms such as “an FLT3-targeting agent” refer the presence of an FLT3-targeting agent but do not exclude the possibility that additional FLT3-targeting agents, e.g. one, two, three or more additional FLT3-targeting agents could be present. In one embodiment in accordance with the invention, only one FLT3-targeting agent is used.

In one embodiment, the chimeric antigen receptor is capable of binding to FLT3. In a preferred embodiment, the chimeric antigen receptor is capable of binding to the extracellular domain of FLT3. In a preferred embodiment, the chimeric antigen receptor is expressed in immune cells, preferably T cells. In a preferred embodiment of the invention, the chimeric antigen receptor is expressed in T cells and allows said T cells to bind specifically to FLT3-expressing acute myeloid leukemia cells with high specificity to exert a growth inhibiting effect, preferably a cytotoxic effect, on said acute myeloid leukemia cells.

In a preferred embodiment, the chimeric antigen receptor capable of binding to FLT3 is a chimeric antigen receptor derived from an antigen-binding portion of a monoclonal antibody capable of binding FLT3, wherein the chimeric antigen receptor comprises the amino acid sequence of SEQ ID NO: 2 or a sequence that is at least 85%, at least 90%, at least 91%, at least 92%, at least 93%, at least 94%, at least 95%, at least 96%, at least 97%, at least 98% or at least 99% identical thereto.

In a more preferred embodiment, the chimeric antigen receptor capable of binding to FLT3 is a chimeric antigen receptor wherein the antigen-binding domain thereof comprises a heavy chain variable domain which comprises the amino acid sequence of SEQ ID NO: 5, and a light chain variable domain which comprises the amino acid sequence of SEQ ID NO: 6. In a preferred embodiment, the chimeric antigen receptor capable of binding to FLT3 is a chimeric antigen receptor derived from an antigen-binding portion of a monoclonal antibody capable of binding FLT3, wherein the chimeric antigen receptor comprises the amino acid sequence of SEQ ID NO: 4 or a sequence that is at least 85%, at least 90%, at least 91%, at least 92%, at least 93%, at least 94%, at least 95%, at least 96%, at least 97%, at least 98% or at least 99% identical thereto.

In a more preferred embodiment, the chimeric antigen receptor capable of binding to FLT3 is a chimeric antigen receptor wherein the antigen-binding domain thereof comprises a heavy chain variable domain which comprises the amino acid sequence of SEQ ID NO: 7, and a light chain variable domain which comprises the amino acid sequence of SEQ ID NO: 8.

“Adoptive immunotherapy” as described herein refers to the transfer of immune cells into a patient for targeted treatment of cancer. The cells may have originated from the patient or from another individual. In adoptive immunotherapy, immune cells, preferably T cells, are typically extracted from an individual, preferably from the patient, genetically modified and cultured in vitro and administered to the patient. Adoptive immunotherapy is advantageous in that it allows targeted growth inhibiting, preferably cytotoxic, treatment of tumor cells without the non-targeted toxicity to non-tumor cells that occurs with conventional treatments.

In a preferred embodiment in accordance with the invention, T cells are isolated from a patient having acute myeloid leukemia, transduced with a gene transfer vector encoding a chimeric antigen receptor capable of binding to FLT3, and administered to the patient to treat acute myeloid leukemia, preferably wherein the acute myeloid leukemia cells expressed mutated FLT3, more preferably FLT3-ITD. In a preferred embodiment, the T cells are CD8⁺ T cells or CD4⁺ T cells.

The term antibody as used herein refers to any functional antibody that is capable of specific binding to the antigen of interest. Without particular limitation, the term antibody encompasses antibodies from any appropriate source species, including avian such as chicken and mammalian such as mouse, goat, non-human primate and human. Preferably, the antibody is a humanized antibody. Humanized antibodies are antibodies which contain human sequences and a minor portion of non-human sequences which confer binding specificity to an antigen of interest (e.g. human FLT3). The antibody is preferably a monoclonal antibody which can be prepared by methods well-known in the art. The term antibody encompasses an IgG-1, -2, -3, or -4, IgE, IgA, IgM, or IgD isotype antibody. The term antibody encompasses monomeric antibodies (such as IgD, IgE, IgG) or oligomeric antibodies (such as IgA or IgM). The term antibody also encompasses—without particular limitations—isolated antibodies and modified antibodies such as genetically engineered antibodies, e.g. chimeric antibodies or bispecific antibodies.

An antibody fragment or fragment of an antibody as used herein refers to a portion of an antibody that retains the capability of the antibody to specifically bind to the antigen (e.g. human FLT3). This capability can, for instance, be determined by determining the capability of the antigen-binding portion to compete with the antibody for specific binding to the antigen by methods known in the art. Without particular limitation, the antibody fragment can be produced by any suitable method known in the art, including recombinant DNA methods and preparation by chemical or enzymatic fragmentation of antibodies. Antibody fragments may be Fab fragments, F(ab′) fragments, F(ab′)2 fragments, single chain antibodies (scFv), single-domain antibodies, diabodies or any other portion(s) of the antibody that retain the capability of the antibody to specifically bind to the antigen.

An “antibody” (e.g. a monoclonal antibody) or “a fragment thereof” as described herein may have been derivatized or be linked to a different molecule. For example, molecules that may be linked to the antibody are other proteins (e.g. other antibodies), a molecular label (e.g. a fluorescent, luminescent, colored or radioactive molecule), a pharmaceutical and/or a toxic agent. The antibody or antigen-binding portion may be linked directly (e.g. in form of a fusion between two proteins), or via a linker molecule (e.g. any suitable type of chemical linker known in the art).

The term “internal tandem duplication” (ITD) as used herein in connection with FLT3 refers to a genetic mutation in FLT3 leading to one or more in-frame trinucleotide duplication in the juxtamembrane region or in other parts of the intracellular domain (FLT3-ITD). This typically results in the constitutive activation of FLT3. Internal tandem duplications can range in size from 3 nucleotides to more than 100 nucleotides. FLT3-ITD mutations occur frequently in acute myeloid leukemia and are associated with resistance to conventional therapy and poor clinical outcome.

Unless specified otherwise, “monotherapy” as described herein means a therapy in which one pharmaceutically active substance, molecule, compound, composition, or agent is administered as the only pharmaceutically active substance, molecule, compound, composition, or agent. The term monotherapy as used herein does not encompass the combined use of two or more pharmaceutically active substances, molecules, compounds, compositions, or agents. The term monotherapy further does not encompass the combined use of two or more pharmaceutically active substances, molecules, compounds, compositions, or agents, where the two or more pharmaceutically active substances, molecules, compounds, compositions, or agents are not administered simultaneously, but are administered within one therapeutic regimen.

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression.

A treatment of cancer according to the present invention does not exclude that additional or secondary therapeutic benefits also occur in patients. For example, an additional or secondary benefit may be an enhancement of engraftment of transplanted hematopoietic stem cells that is carried out prior to, concurrently to, or after the treatment of cancer. However, it is understood that the primary treatment for which protection is sought is for treating the cancer itself, and any secondary or additional effects only reflect optional, additional advantages of the treatment of cancer growth.

The treatment of cancer according to the invention can be a first-line therapy, a second-line therapy, a third-line therapy, or a fourth-line therapy. The treatment can also be a therapy that is beyond is beyond fourth-line therapy. The meaning of these terms is known in the art and in accordance with the terminology that is commonly used by the US National Cancer Institute.

The term “refractory to induction chemotherapy” as used herein refers to patients whose disease did not respond to one or two cycles of induction chemotherapy.

The term “capable of binding” as used herein refers to the capability to form a complex with a molecule that is to be bound (e.g. FLT3). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in K_{D} values is preferably is less than 1 μM, more preferably less than 100 nM, even more preferably less than 10 nM, even more preferably less than 1 nM, even more preferably less than 100 pM, even more preferably less than 10 pM, even more preferably less than 1 pM.

As used herein, each occurrence of terms such as “comprising” or “comprises” may optionally be substituted with “consisting of” or “consists of”.

A pharmaceutically acceptable carrier, including any suitable diluent or, can be used herein as known in the art. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

Compositions and formulations in accordance with the present invention are prepared in accordance with known standards for the preparation of pharmaceutical compositions and formulations. For instance, the compositions and formulations are prepared in a way that they can be stored and administered appropriately, e.g. by using pharmaceutically acceptable components such as carriers, excipients or stabilizers. Such pharmaceutically acceptable components are not toxic in the amounts used when administering the pharmaceutical composition or formulation to a patient. The pharmaceutical acceptable components added to the pharmaceutical compositions or formulations may depend on the chemical nature of the inhibitor and targeting agent present in the composition or formulation (depend on whether the targeting agent is e.g. an antibody or fragment thereof or a cell expressing a chimeric antigen receptor), the particular intended use of the pharmaceutical compositions and the route of administration.

In a preferred embodiment in accordance with the invention, the composition or formulation is suitable for administration to humans, preferably the formulation is sterile and/or non-pyrogenic.

A preferred embodiment is the use of FLT3 CAR-T cells in combination with crenolanib to treat FLT3-ITD+AML.

Another useful embodiment is the use of FLT3 CAR-T cells in combination with crenolanib to treat FLT3-mutated (any other mutation than FLT3-ITD) or FLT3 wild-type AML.

Another useful embodiment is the use of FLT3 CAR-T cells in combination with midostaurin, quizartinib, or any other FLT3 inhibitor to treat FLT3-ITD+, FLT3-mutated or FLT3 wild-type AML.

Another useful embodiment is the use of FLT3 CAR-T cells in combination with one or several FLT3 inhibitors to treat FLT3-ITD+, FLT3-mutated or FLT3 wild-type AML.

Another useful embodiment is the use of FLT3 CAR-T cells in combination with one or several multikinase inhibitors to treat FLT3-ITD+, FLT3-mutated or FLT3 wild-type AML.

A preferred embodiment is the use of autologous FLT3 CAR-T cells in combination with crenolanib to treat FLT3-ITD+AML.

Another useful embodiment is the use of allogeneic FLT3 CAR-T cells in combination with crenolanib to treat FLT3-ITD+AML.

In a preferred embodiment autologous FLT3 CAR-T cells are administered in combination with crenolanib prior to an allogeneic hematopoietic stem cell transplantation to treat FLT3-ITD+AML.

In another useful embodiment autologous FLT3 CAR-T cells are administered in combination with crenolanib after an allogeneic hematopoietic stem cell transplantation to treat FLT3-ITD+AML.

In a useful embodiment allogeneic FLT3 CAR-T cells are administered in combination with crenolanib prior to an allogeneic hematopoietic stem cell transplantation to treat FLT3-ITD+AML.

In another useful embodiment allogeneic FLT3 CAR-T cells are administered in combination with crenolanib after an allogeneic hematopoietic stem cell transplantation to treat FLT3-ITD+AML.

In a preferred embodiment, CD8+ and CD4+FLT3 CAR-T cells are administered in combination with crenolanib to treat FLT3-ITD+AML.

In another useful embodiment, only CD8+FLT3 CAR-T cells are administered in combination with crenolanib to treat FLT3-ITD+AML.

In another useful embodiment, only CD4+FLT3 CAR-T cells are administered in combination with crenolanib to treat FLT3-ITD+AML.

In other useful embodiments, any other T cell (including but not limited to: naïve T cell, memory T cell, memory stem T cell, gamma delta T cell, cytokine-induced killer cell, regulatory T cell), NK cell or B-cell modified with the FLT3 CAR is used in combination with crenolanib to treat FLT3-ITD+AML.

In a preferred embodiment the FLT3 CAR is expressed in CD8+ and CD4+ T cells through stable gene transfer, wherein the stable gene transfer is accomplished through viral vectors or non-viral gene transfer.

In another preferred embodiment the FLT3 CAR is expressed in CD8+ and CD4+ T cells though transient gene transfer or any other means resulting in transient expression of the FLT3 CAR protein.

Other preferred embodiments include the use of FLT3-specific antibodies (including but not limited to: monoclonal antibodies, bi-specific antibodies, tri-specific antibodies, antibody-drug conjugates) in combination with crenolanib to treat FLT3-ITD+AML.

Another useful embodiment is the use of FLT3 CAR-T cells in combination with crenolanib to treat acute lymphoblastic leukemia. Another useful embodiment is the use of FLT3 CAR-T cells in combination with crenolanib to treat mixed lineage leukemia, myeloid dysplastic syndrome, or any other cancer expressing FLT3.

Another useful embodiment is the use of FLT3 CAR-T cells in combination with crenolanib to eliminate leukemic stem/initiating cells.

Another useful embodiment is the use of FLT3 CAR-T cells in combination with crenolanib to eliminate hematopoietic stem cells, hematopoietic progenitor cells, NK cells, dendritic cells.

FLT3 Targeting Agents and their Use According to the Invention

An FLT3 targeting agent according to the invention can be any agent capable of specifically binding to its target, wherein the target is FLT3, preferably a cell expressing FLT3 on its cell surface, and wherein the FLT3 targeting agent promotes the targeted treatment of FLT3 expressing cell types without the risk of affecting other cell types.

A non-limiting example of an FLT3 targeting agent is a T cell expressing a chimeric antigen receptor capable of specifically binding FLT3 (a FLT3 CAR-T cell) thus capable of targeting acute myeloid tumor cells expressing FLT3.

Whether or not a targeting agent is an FLT3 targeting agent can be determined by using the methods disclosed herein, as detailed in the preferred embodiments. A preferred method in accordance with the preferred embodiments is the method used in Examples 1 and 2.

In one embodiment, the FLT3 targeting agent is a T cell expressing a chimeric antigen receptor capable of binding to FLT3 (FLT3 CAR-T cell).

In another embodiment, the FLT3 targeting agent is a FLT3 CAR-T cell, wherein said FLT3 CAR-T cell is administered to a patient in need thereof in a method for the treatment of cancer, preferably for the treatment of leukemia or lymphoma, more preferably for the treatment of leukemia, most preferably for the treatment of acute myeloid leukemia.

In a preferred embodiment, the FLT3 targeting agent is a FLT3 CAR-T cell, wherein said FLT3 CAR-T cell is administered to a patient in need thereof in a method for the treatment of acute myeloid leukemia, wherein the acute myeloid leukemia tumor cells express FLT3, preferably mutated FLT3, more preferably FLT3-ITD.

In a more preferred embodiment, the FLT3 targeting agent is a T cell expressing a chimeric antigen receptor capable of binding to FLT3, wherein said chimeric antigen receptor is a chimeric antigen receptor wherein the antigen-binding domain thereof comprises a heavy chain variable domain which comprises the amino acid sequence of SEQ ID NO: 5, and a light chain variable domain which comprises the amino acid sequence of SEQ ID NO: 6 and is administered to a patient in need thereof in a method for the treatment of acute myeloid leukemia, wherein the acute myeloid leukemia tumor cells express FLT3, preferably mutated FLT3, more preferably FLT3-ITD.

In a more preferred embodiment, the FLT3 targeting agent is a is a T cell expressing a chimeric antigen receptor capable of binding to FLT3, wherein said chimeric antigen receptor is a chimeric antigen receptor wherein the antigen-binding domain thereof comprises a heavy chain variable domain which comprises the amino acid sequence of SEQ ID NO: 7, and a light chain variable domain which comprises the amino acid sequence of SEQ ID NO: 8 and is administered to a patient in need thereof in a method for the treatment of acute myeloid leukemia, wherein the acute myeloid leukemia tumor cells express FLT3, preferably mutated FLT3, more preferably FLT3-ITD.

In a more preferred embodiment, the FLT3 targeting agent is a is a T cell expressing a chimeric antigen receptor capable of binding to FLT3, wherein said chimeric antigen receptor is a chimeric antigen receptor comprising the amino acid sequence of SEQ ID NO: 2 or a sequence that is at least 85%, at least 90%, at least 91%, at least 92%, at least 93%, at least 94%, at least 95%, at least 96%, at least 97%, at least 98% or at least 99% identical thereto, and is administered to a patient in need thereof in a method for the treatment of acute myeloid leukemia, wherein the acute myeloid leukemia tumor cells express FLT3, preferably mutated FLT3, more preferably FLT3-ITD.

In a more preferred embodiment, the FLT3 targeting agent is a is a T cell expressing a chimeric antigen receptor capable of binding to FLT3, wherein said chimeric antigen receptor is a chimeric antigen receptor comprising the amino acid sequence of SEQ ID NO: 4 or a sequence that is at least 85%, at least 90%, at least 91%, at least 92%, at least 93%, at least 94%, at least 95%, at least 96%, at least 97%, at least 98% or at least 99% identical thereto, and is administered to a patient in need thereof in a method for the treatment of acute myeloid leukemia, wherein the acute myeloid leukemia tumor cells express FLT3, preferably mutated FLT3, more preferably FLT3-ITD.

In an even more preferred embodiment, the FLT3 targeting agent is a is a T cell, preferably a CD8⁺ T cell or a CD4⁺ T cell, expressing a chimeric antigen receptor capable of binding to FLT3, wherein said chimeric antigen receptor is a chimeric antigen receptor comprising the amino acid sequence of SEQ ID NO: 2 or SEQ ID NO: 4, and is administered to a patient in need thereof in a method for the treatment of acute myeloid leukemia, wherein the acute myeloid leukemia tumor cells express FLT3, preferably mutated FLT3, more preferably FLT3-ITD.

In one embodiment, the kinase inhibitor is a tyrosine kinase inhibitor, preferably a receptor tyrosine kinase inhibitor, more preferably an FLT3 inhibitor. FLT3 inhibitors according to the invention can be type I FLT3 inhibitors or type II FLT3 inhibitors. In a preferred embodiment, the FLT3 inhibitor is a type II FLT3 inhibitor, preferably midostaurin or quizartinib. In a more preferred embodiment, the FLT3 inhibitor is a type I FLT3 inhibitor, preferably crenolanib.

In a preferred embodiment, the kinase inhibitor is an FLT3 inhibitor and is administered to a patient in need thereof in a method for the treatment of acute myeloid leukemia, wherein the acute myeloid leukemia cells express FLT3, preferably mutated FLT3, more preferably FLT3-ITD.

In a more preferred embodiment, the kinase inhibitor is an FLT3 inhibitor, preferably midostaurin or quizartinib, more preferably crenolanib, and is administered to a patient in need thereof in a method for the treatment of acute myeloid leukemia, wherein the acute myeloid leukemia cells express FLT3, preferably mutated FLT3, more preferably FLT3-ITD, and wherein the expression of FLT3 is upregulated upon administration of said FLT3 inhibitor.

Therapeutic Methods and Products for Use in these Methods

The present invention relates to FLT3 targeting agents and kinase inhibitors and their use in the treatment of acute myeloid leukemia as described above.

Additionally, and in accordance with these FLT3 targeting agents and their uses, the present invention also relates to corresponding therapeutic methods.

In one embodiment, the invention relates to a method for administering an FLT3 targeting agent in combination with a kinase inhibitor to a patient in a method for treatment of acute myeloid leukemia.

In a more preferred embodiment, the invention relates to administering an FLT3 targeting agent to a patient having cancer in need thereof, wherein the FLT3 targeting agent is a T cell expressing a chimeric antigen receptor capable of binding FLT3 (FLT3 CAR-T cell), wherein the chimeric antigen receptor comprises a heavy chain variable domain comprising the amino acid sequence of SEQ ID NO: 5 or SEQ ID NO: 7, and a light chain variable domain which comprises the amino acid sequence of SEQ ID NO: 6 or SEQ ID NO: 8, in combination with a kinase inhibitor, wherein the kinase inhibitor is an FLT3 inhibitor, preferably quizartinib or midostaurin, more preferably crenolanib, wherein the cancer is acute myeloid leukemia, wherein the acute myeloid leukemia tumor cells express FLT3, preferably mutated FLT3, more preferably FLT3-ITD.

In a preferred embodiment, the invention relates to administering an FLT3 targeting agent to a patient having cancer in need thereof, wherein the FLT3 targeting agent is a T cell expressing a chimeric antigen receptor capable of binding FLT3 (FLT3 CAR-T cell), in combination with a kinase inhibitor, wherein the kinase inhibitor is an FLT3 inhibitor, the cancer is acute myeloid leukemia, and wherein the acute myeloid leukemia tumor cells express FLT3.

In a more preferred embodiment, the invention relates to administering an FLT3 targeting agent to a patient having cancer in need thereof, wherein the FLT3 targeting agent is a T cell expressing a chimeric antigen receptor capable of binding FLT3 (FLT3 CAR-T cell), wherein the chimeric antigen receptor comprises a heavy chain variable domain comprising the amino acid sequence of SEQ ID NO: 5 or SEQ ID NO: 7, and a light chain variable domain which comprises the amino acid sequence of SEQ ID NO: 6 or SEQ ID NO: 8, in combination with a kinase inhibitor, wherein the kinase inhibitor is an FLT3 inhibitor, preferably quizartinib or midostaurin, more preferably crenolanib, wherein the cancer is acute myeloid leukemia, wherein the acute myeloid leukemia tumor cells express FLT3, preferably wherein the tumor cells express mutated FLT3, more preferably FLT3-ITD.

In an even more preferred embodiment, the invention relates to administering a kinase inhibitor to a patient having cancer in need thereof, wherein the kinase inhibitor is an FLT3 inhibitor, preferably quizartinib or midostaurin, more preferably crenolanib, wherein the cancer is acute myeloid leukemia, wherein the acute myeloid leukemia tumor cells express FLT3, preferably mutated FLT3, more preferably FLT3-ITD, wherein the FLT3 inhibitor is administered prior to, concurrently to, or after the administration of an FLT3 targeting agent, which causes an upregulation of FLT3 expression and an increased antigen density on the tumor cell surface, wherein said antigen is part of the FLT3 extracellular domain. In this embodiment, the FLT3-targeting agent to be administered prior to, concurrently to, or after the administration of the FLT3 inhibitor is a T cell expressing a chimeric antigen receptor capable of binding FLT3 (FLT3 CAR-T cell), preferably wherein the chimeric antigen receptor comprises a heavy chain variable domain comprising the amino acid sequence of SEQ ID NO: 5 or SEQ ID NO: 7, and a light chain variable domain which comprises the amino acid sequence of SEQ ID NO: 6 or SEQ ID NO: 8, wherein the antigen the FLT3 CAR-T cell binds to is part of the FLT3 extracellular domain, of which the FLT3 inhibitor causes upregulation and increased antigen density in the acute myeloid leukemia tumor cells. Thus, according to the embodiment, the combined administration of an FLT3 inhibitor, in which the FLT3 inhibitor causes upregulation of FLT3 and increased antigen density of the FLT3 extracellular domain on the cell surface of the acute myeloid tumor cells, and of an FLT3 targeting agent that is an FLT3 CAR-T cell binding to said FLT3 extracellular domain leads to an improvement in acute myeloid leukemia therapy compared to monotherapy with either the FLT3 inhibitor or the FLT3 CAR-T cells alone. Therefore, according to this embodiment the combined administration of an FLT3 inhibitor and an FLT3 targeting agent which is an FLT3 CAR-T cell achieves a surprising and unexpected synergistic effect which provides an improvement in the treatment of acute myeloid leukemia.

In another embodiment, the invention relates to administering an FLT3 targeting agent, wherein the FLT3 targeting agent is an antibody or fragment thereof capable of binding FLT3, in combination with a kinase inhibitor, wherein the kinase inhibitor is an FLT3 inhibitor, to a patient having acute myeloid leukemia, wherein the acute myeloid leukemia tumor cells express FLT3.

In a more preferred embodiment, the invention relates to administering an FLT3 targeting agent, wherein the FLT3 targeting agent is an antibody or fragment thereof, wherein the antibody or fragment thereof comprises a heavy chain variable domain comprising the amino acid sequence of SEQ ID NO: 5 or SEQ ID NO: 7, and a light chain variable domain which comprises the amino acid sequence of SEQ ID NO: 6 or SEQ ID NO: 8, in combination with a kinase inhibitor, wherein the kinase inhibitor is an FLT3 inhibitor, preferably quizartinib or midostaurin, more preferably crenolanib, to a patient having acute myeloid leukemia, wherein the acute myeloid leukemia tumor cells express FLT3, preferably wherein the tumor cells express mutated FLT3, more preferably FLT3-ITD.

FLT3 Targeting Agents and their Use in Combination with Kinase Inhibitors According to the Invention

The present invention encompasses combinations of an FLT3 targeting agent and a kinase inhibitor for use in a method of treating cancer in a human patient, wherein the FLT3 targeting agent and the kinase inhibitor are to be administered to the human patient in combination.

### SEQUENCES

The amino acid sequences referred to in the present application are as follows (in an N-terminal to C-terminal order; represented in the one-letter amino acid code):

SEQ ID No: 2 (Sequence of 4G8 FLT3 CAR):
QVQLQQPGAELVKPGASLKLSCKSSGYTFTSYWMHWVRQRPGHGLEWIGE
IDPSDSYKDYNQKFKDKATLTVDRSSNTAYMHLSSLTSDDSAVYYCARAI
TTTPFDFWGQGTTLTVSSGGGGSGGGGSGGGGSDIVLTQSPATLSVTPGD
SVSLSCRASQSISNNLHWYQQKSHESPRLLIKYASQSISGIPSRFSGSGS
GTDFTLSINSVETEDFGVYFCQQSNTWPYTFGGGTKLEIKRESKYGPPCP
PCPMFWVLVVVGGVLACYSLLVTVAFIIFWVRSKRSRGGHSDYMNMTPRR
PGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR
SEQ ID No: 4 (Sequence of BV10 FLT3 CAR):
QVQLKQSGPGLVQPSQSLSITCTVSGFSLTNYGLHWVRQSPGKGLEWLGV
IWSGGSTDYNAAFISRLSISKDNSKSQVFFKMNSLQADDTAIYYCARKGG
IYYANHYYAMDYWGQGTSVTVSSGGGGSGGGGSGGGGSDIVMTQSPSSLS
VSAGEKVTMSCKSSQSLLNSGNQKNYMAWYQQKPGQPPKLLIYGASTRES
GVPDRFTGSGSGTDFTLTISSVQAEDLAVYYCQNDHSYPLTFGAGTKLEL
KRESKYGPPCPPCPMFWVLVVVGGVLACYSLLVTVAFIIFWVRSKRSRGG
HSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 5 (4G8 heavy chain variable domain
(VH)):
QVQLQQPGAELVKPGASLKLSCKSSGYTFTSYWMHWVRQRPGHGLEWIGE
IDPSDSYKDYNQKFKDKATLTVDRSSNTAYMHLSSLTSDDSAVYYCARAI
TTTPFDFWGQGTTLIVSS
SEQ ID NO: 6 (4G8 light chain variable domain
(VH)):
DIVLTQSPATLSVTPGDSVSLSCRASQSISNNLHWYQQKSHESPRLLIKY
ASQSISGIPSRFSGSGSGTDFTLSINSVETEDFGVYFCQQSNTWPYTFGG
GTKLEIKR
SEQ ID NO: 7 (BV10 heavy chain variable domain
(VH)):
QVQLKQSGPGLVQPSQSLSITCTVSGFSLTNYGLHWVRQSPGKGLEWLGV
IWSGGSTDYNAAFISRLSISKDNSKSQVFFKMNSLQADDTAIYYCARKGG
IYYANHYYAMDYWGQGTSVTVSS
SEQ ID NO: 8 (BV10 light chain variable domain
(VH)):
DIVMTQSPSSLSVSAGEKVTMSCKSSQSLLNSGNQKNYMAWYQQKPGQPP
KLLIYGASTRESGVPDRFTGSGSGTDFTLTISSVQAEDLAVYYCQNDHSY
PLTFGAGTKLELKR
SEQ ID NO: 9 (GMCSF signal peptide):
MLLLVTSLLLCELPHPAFLLIP
SEQ ID NO: 10 (4(GS)x3 linker):
GGGGSGGGGSGGGGS
SEQ ID NO: 11 (IgG4 hinge domain):
ESKYGPPCPPCP
SEQ ID NO: 12 (CD28 transmembrane domain):
MFWVLVVVGGVLACYSLLVTVAFIIFWV
SEQ ID NO: 13 (CD28 costimulatory domain):
RSKRSRGGHSDYMNMTPRRPGPTRKHYQPYAPPRDFAAYRS
SEQ ID NO: 14 (CD3z signaling domain):
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
SEQ ID NO: 15 (T2A ribosomal skipping sequence):
LEGGGEGRGSLLTCGDVEENPGPR
SEQ ID NO: 16 (EGFRt):
RKVCNGIGIGEFKDSLSINATNIKHFKNCTSISGDLHILPVAFRGDSFTH
TPPLDPQELDILKTVKEITGFLLIQAWPENRTDLHAFENLEIIRGRTKQH
GQFSLAVVSLNITSLGLRSLKEISDGDVIISGNKNLCYANTINWKKLFGT
SGQKTKIISNRGENSCKATGQVCHALCSPEGCWGPEPRDCVSCRNVSRGR
ECVDKCNLLEGEPREFVENSECIQCHPECLPQAMNITCTGRGPDNCIQCA
HYIDGPHCVKTCPAGVMGENNTLVWKYADAGHVCHLCHPNCTYGCTGPGL
EGCPTNGPKIPSIATGMVGALLLLLVVALGIGLFM

The nucleic acid sequences referred to in the present application are as follows (from 5′ to 3′; represented in accordance with the standard nucleic acid code):

SEQ ID No: 1 (Sequence of 4G8 FLT3 CAR):
CAGGTGCAGCTGCAGCAGCCTGGCGCCGAACTCGTGAAACCTGGCGCCTC
TCTGAAGCTGAGCTGCAAGAGCAGCGGCTACACCTTCACCAGCTACTGGA
TGCACTGGGTGCGCCAGAGGCCTGGCCACGGACTGGAATGGATCGGCGAG
ATCGACCCCAGCGACAGCTACAAGGACTACAACCAGAAGTTCAAGGACAA
GGCCACCCTGACCGTGGACAGAAGCAGCAACACCGCCTACATGCACCTGT
CCAGCCTGACCAGCGACGACAGCGCCGTGTACTACTGTGCCAGAGCCATC
ACAACCACCCCCTTCGATTTCTGGGGCCAGGGCACAACCCTGACAGTGTC
TAGCGGAGGCGGAGGCTCCGGAGGGGGAGGATCTGGGGGAGGCGGAAGCG
ATATTGTGCTGACCCAGAGCCCTGCCACACTGAGCGTGACACCAGGCGAT
AGCGTGTCCCTGTCCTGCAGAGCCAGCCAGAGCATCTCCAACAACCTGCA
CTGGTATCAGCAGAAGTCCCACGAGAGCCCCAGACTGCTGATTAAGTACG
CCAGCCAGTCCATCAGCGGCATCCCCAGCAGATTTTCCGGCAGCGGCTCC
GGCACCGACTTCACCCTGAGCATCAACAGCGTGGAAACCGAGGACTTCGG
CGTGTACTTCTGCCAGCAGAGCAACACCTGGCCTTACACCTTCGGCGGAG
GCACCAAGCTGGAAATCAAGAGAGAGTCTAAGTACGGACCGCCCTGCCCC
CCTTGCCCTATGTTCTGGGTGCTGGTGGTGGTCGGAGGCGTGCTGGCCTG
CTACAGCCTGCTGGTCACCGTGGCCTTCATCATTTTTGGGTCCGCAGCAA
GCGGAGCAGAGGCGGCCACAGCGACTACATGAACATGACCCCTAGACGGC
CTGGCCCCACCAGAAAGCACTACCAGCCCTACGCCCCTCCCCGGGACTTT
GCCGCCTACAGAAGCCGGGTGAAGTTCAGCAGAAGCGCCGACGCCCCTGC
CTACCAGCAGGGCCAGAATCAGCTGTACAACGAGCTGAACCTGGGCAGAA
GGGAAGAGTACGACGTCCTGGATAAGCGGAGAGGCCGGGACCCTGAGATG
GGCGGCAAGCCTCGGCGGAAGAACCCCCAGGAAGGCCTGTATAACGAACT
GCAGAAAGACAAGATGGCCGAGGCCTACAGCGAGATCGGCATGAAGGGCG
AGCGGAGGCGGGGCAAGGGCCACGACGGCCTGTATCAGGGCCTGTCCACC
GCCACCAAGGATACCTACGACGCCCTGCACATGCAGGCCCTGCCCCCAAG
G
SEQ ID No: 3 (Sequence of BV10 FLT3 CAR):
CAGGTGCAGCTGAAGCAGAGCGGCCCTGGACTGGTGCAGCCTAGCCAGAG
CCTGAGCATCACCTGTACCGTGTCCGGCTTCAGCCTGACCAACTACGGCC
TGCATTGGGTGCGCCAGAGCCCTGGCAAAGGCCTGGAATGGCTGGGAGTG
ATTTGGAGCGGCGGCAGCACCGACTACAACGCCGCCTTCATCAGCAGACT
GAGCATCTCCAAGGACAACAGCAAGAGCCAGGTGTTCTTCAAGATGAACT
CCCTGCAGGCCGACGACACCGCCATCTACTACTGCGCCAGAAAGGGCGGC
ATCTACTATGCCAACCACTACTACGCTATGGACTACTGGGGCCAGGGCAC
CAGCGTGACAGTGTCTAGCGGAGGCGGAGGCTCCGGAGGGGGAGGATCTG
GGGGAGGCGGATCTGACATCGTGATGACCCAGAGCCCCAGCAGCCTGTCT
GTGTCTGCCGGCGAGAAAGTGACCATGAGCTGCAAGAGCAGCCAGTCCCT
GCTGAACAGCGGCAACCAGAAAAACTACATGGCCTGGTATCAGCAGAAGC
CCGGCCAGCCCCCTAAGCTGCTGATCTACGGCGCCAGCACCAGAGAAAGC
GGCGTGCCCGATAGATTCACCGGCAGCGGCTCTGGCACCGACTTTACCCT
GACCATCAGCAGCGTGCAGGCTGAGGACCTGGCCGTGTACTACTGCCAGA
ACGACCACAGCTACCCCCTGACCTTTGGAGCCGGCACCAAGCTGGAACTG
AAGAGAGAGTCTAAGTACGGACCGCCCTGCCCCCCTTGCCCTATGTTCTG
GGTGCTGGTGGTGGTCGGAGGCGTGCTGGCCTGCTACAGCCTGCTGGTCA
CCGTGGCCTTCATCATCTTTTGGGTCCGCAGCAAGCGGAGCAGAGGCGGC
CACAGCGACTACATGAACATGACCCCTAGACGGCCTGGCCCCACCAGAAA
GCACTACCAGCCCTACGCCCCTCCCCGGGACTTTGCCGCCTACAGAAGCC
GGGTGAAGTTCAGCAGAAGCGCCGACGCCCCTGCCTACCAGCAGGGCCAG
AATCAGCTGTACAACGAGCTGAACCTGGGCAGAAGGGAAGAGTACGACGT
CCTGGATAAGCGGAGAGGCCGGGACCCTGAGATGGGCGGCAAGCCTCGGC
GGAAGAACCCCCAGGAAGGCCTGTATAACGAACTGCAGAAAGACAAGATG
GCCGAGGCCTACAGCGAGATCGGCATGAAGGGCGAGCGGAGGCGGGGCAA
GGGCCACGACGGCCTGTATCAGGGCCTGTCCACCGCCACCAAGGATACCT
ACGACGCCCTGCACATGCAGGCCCTGCCCCCAAGG

### EXAMPLES

Additional aspects and details of the invention are exemplified by the following non-limiting examples.

### Example 1

Materials and Methods

Human Subjects

Peripheral blood was obtained from healthy donors and adult AML patients after written informed consent to participate in research protocols approved by the Institutional Review Board of the participating institutions.

Primary AML Cells

Primary AML cells were maintained in RPMI-1640 supplemented with 10% human serum, 2 mM glutamine, 100 U/mL penicillin/streptomycin, and a cytokine cocktail including IL-4 (1000 IU/mL), granulocyte macrophage colony-stimulating factor (GM-CSF) (10 ng/mL), stem cell factor (5 ng/mL) and tumor necrosis factor (TNF)-α (10 ng/mL).

Tumor Cell Lines

The human leukemia cell lines MOLM-13 (ACC 554), THP-1 (ACC 16), MV4;11 (ACC 102), and K562 (ACC 10) were purchased from DSMZ (Deutsche Sammlung von Mikroorganismen and Zellkulturen, Braunschweig, Germany) and cultured in RPMI-1640 supplemented with 10% fetal calf serum (FCS), 2 mM glutamine and 100 U/mL penicillin/streptomycin. All cell lines were transduced with a lentiviral vector encoding a firefly luciferase (ffluc)_green fluorescent protein (GFP) transgene to enable detection by flow cytometry (GFP) and bioluminescence imaging (ffLuc) in mice, and bioluminescence-based cytotoxicity assays. K562/FLT3 was generated by retroviral transduction with the full-length human FLT3 gene.

Flow Cytometric Analysis of FLT3 Expression

Cell surface expression of FLT3 (CD135) was analyzed using a conjugated mouse-anti-human-FLT3 mAb (clone 4G8, BD Pharmagin, BD Biosciences, Germany) and mouse IgG1 isotype control (BD Pharmagin). In brief, 1×10⁶ cells were washed, resuspended in 100 μl. PBS/0.5% fetal calf serum and stained with 5 μL of anti-FLT3 mAb or isotype for 30 minutes at 4° C.

CAR Construction

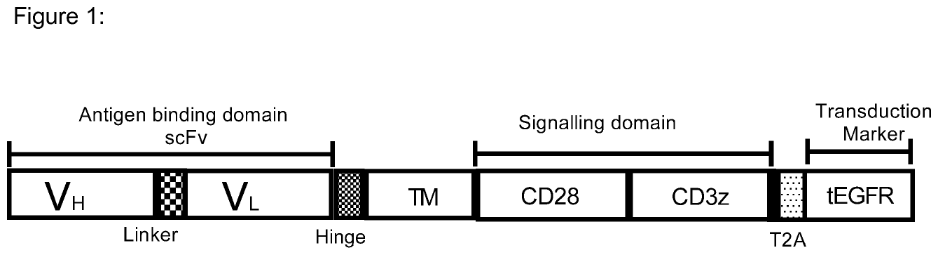

A codon optimized targeting domain comprising the V_{H} and V_{L} segments of the FLT3-specific 4G8 mAb¹² was synthesized (GeneArt, ThermoFisher, Regensburg, Germany) and fused to a CAR backbone comprising a short IgG4-Fc Hinge spacer, a CD28 transmembrane and costimulatory moiety and CD3z, in-frame with a T2A element and EGFRt transduction marker (FIG. 1)³²⁻³⁴. The entire transgene was encoded in a lentiviral vector epHIV7 and expressed under control of an EF1/HTLV hybrid promotor^{34, 35}. Similarly, targeting domains specific for CD19 (clone FMC63) and CD123 (clone 32716) were used to generate CD19 and CD123 CARs, respectively^{32, 33, 36, 37}.

EGFR Preparation of CAR-Modified T Cells

Lentiviral gene-transfer was performed into CD3/28-bead (ThermoFisher) activated CD4⁺ and CD8⁺ T cells on day 1 after bead stimulation at a moiety of infection (MOI) of 5. T cells were cultured in RPMI-1640 supplemented with 10% human serum, glutamine, 2 mM glutamine, 100 U/mL penicillin/streptomycin and 50 U/mL recombinant human interleukin (IL)-2 (Proleukine, Novartis, Basel, Switzerland)³². CAR-transduced T cells were enriched using biotinylated anti-EGFR mAb (ImClone Systems Inc.) and anti-biotin beads (Miltenyi), prior to expansion using a rapid expansion protocol³⁸ or—for CD19 CAR-T cells—using antigen-specific stimulation with irradiated (80 Gy) CD19⁺ feeder cells³⁸.

Flow Cytometric Analyses of T Cells

Primary AML and peripheral blood mononuclear cells (PBMCs) were stained with 1 or more of the following conjugated mAbs: CD3, CD19, CD34, CD38, CD33, CD45, CD123, CD135 and matched isotype controls (Miltenyi, Bergisch-Gladbach, Germany/BD, Heidelberg, Germany/Biolegend, London, UK). CAR-modified and untransduced T cells were stained with 1 or more of the following conjugated mAbs: CD4, CD8, CD45RA, CD45RO, CD62L, and 7-AAD for live/dead cell discrimination (Miltenyi/BD/Biolegend). CAR-transduced (i.e. EGFRt⁺) T-cells were detected by staining with anti-EGFR antibody that had been biotinylated in-house (EZ-Link™Sulfo-NHS-SS-Biotin, Thermofisher Scientific, IL, according to the manufacturer's instructions) and streptavidin-PE. Flow analyses were done on a FACSCanto (BD) and data analyzed using Flowio software v9.0.2 (Treestar, Ashland, Oreg.).

Analysis of CAR-T Cell Function In Vitro

Functional analyses were performed as previously described^{32, 33, 39-41}. In brief, target cells expressing firefly luciferase (ffLuc) were incubated in triplicate at 5×10³ cells/well with effector T-cells at various effector to target (E:T) ratios. After 4-hour incubation, luciferin substrate was added to the co-culture and the decrease in luminescence signal in wells that contained target cells and T-cells was measured using a luminometer (Tecan, Männedorf, Switzerland) and compared to target cells alone. Specific lysis was calculated using the standard formula⁴². For analysis of cytokine secretion, 50×10³ T-cells were plated in triplicate wells with target cells at a ratio of 2:1 and IFN-γ and IL-2 production measured by ELISA (Biolegend) in supernatant removed after 24-hour incubation. For analysis of proliferation, 50×10³ T-cells were labeled with 0.2 μM carboxyfluorescein succinimidyl ester (CFSE, ThermoFisher), washed and plated in triplicate wells with target cells at a ratio of 2:1 in medium without exogenous cytokines. After 72-hour incubation, cells were labeled with anti-CD8/CD4 mAb and 7-AAD to exclude dead cells from analysis. Samples were analyzed by flow cytometry and division of live T-cells assessed by CFSE dilution. The cytolytic activity of CAR-modified and control T cells against primary AML cells was analyzed in a FACS-based cytoxicity assay. T cells and AML cells were seeded into 96-well plates at effector:target (E:T) ratios ranging from 20:1 to 1:1, with 10×10³ target cells per well. After 4-24 hours, the cultures were aspirated, stained with 7-AAD to discriminate live and dead cells and anti-CD3/anti-CD33/anti-CD45 mAbs to distinguish T cells and AML cells. To quantitate the number of residual life AML cells, 123-counting beads (e-bioscience, San Diego, Calif.) were used according to the manufacturer's instructions. Flow analyses were done on a FACS Canto II (BD) and data analyzed using FlowJo software (Treestar).

In Vivo Experiments

All experiments were approved by the Institutional Animal Care and Use Committees of the participating institutions. NOD.Cg-Prkdc^{scid} Il2rg^{tm1WjI}(NSG) mice (female, 6-8 week old) were purchased from Charles River or bred in-house. Mice were inoculated with 1×10⁶ ffluc_GFP⁺MOLM-13 AML cells by tail vein injection on day 0, and received a single dose of 5×10⁶ T cells (in 200 μl of PBS/0.5% FCS) by tail vein injection on day 7. Crenolanib [15 mg/kg; 200 μL of 30% glycerol formal (Sigma Aldrich, Munich, Germany)] was administered intraperitoneally (i.p.) Monday-Friday for 3 consecutive weeks. AML progression/regression was assessed by serial bioluminescence imaging following i.p. administration of D-luciferin substrate (0.3 mg/g body weight) (Biosynth, Staad, Switzerland) using an IVIS Lumina imaging system (Perkin Elmer, Waltham, Mass.). Data was analyzed using Living Image software (Perkin Elmer).

FLT3 Inhibitor Treatment of MOLM-13 AML Cells

MOLM-13 were maintained in RPMI-1640 medium, supplemented with 10% fetal calf serum, 2 mM glutamine, 100 U/mL penicillin/streptomycin, and 10 nM crenolanib or 1 nM quizartinib or 10 nM midostaurin. A complete medium change was performed every 7 days, MOLM-13 cells adjusted to 1×10⁶/mL medium and 2 mL of this cell suspension plated per well in 48-well plates (Costar, Corning, NJ). After 2-3 weeks of culture with 10 nM midostaurine, MOLM-13 cells were exposed to exponentially increasing concentration of midostaurine for next 8-10 weeks to reach 50 nM midostaurin.

Pharmaceutical Drugs and Reagents

Crenolanib, quizartinib (SelleckChemicals, Houston, Tex.), midostaurin (Novartis, Basel, Switzerland/SelleckChemicals, Houston, Tex./Sigma-Aldrich, Steinheim, Germany) were reconstituted in dimethylsulfoxide (DMSO) prior to dilution in medium or 30% glycerol formal (Sigma Aldrich, Munich, Germany) and use in the in vitro or in vivo experiments, respectively.

Statistical Analyses

Statistical analyses were performed using Prism software v6.07 (GraphPad). Unpaired Student's t-tests were used for analysis of data obtained in in vitro experiments. Log-rank (Mantel-Cox) testing was performed to analyze differences in survival observed in in vivo experiments. Differences with a p value <0.05 were considered statistically significant.

Results:

FLT3 CAR-T Cells Eliminate FLT3 Wild-Type and FLT3-ITD⁺ AML Cells

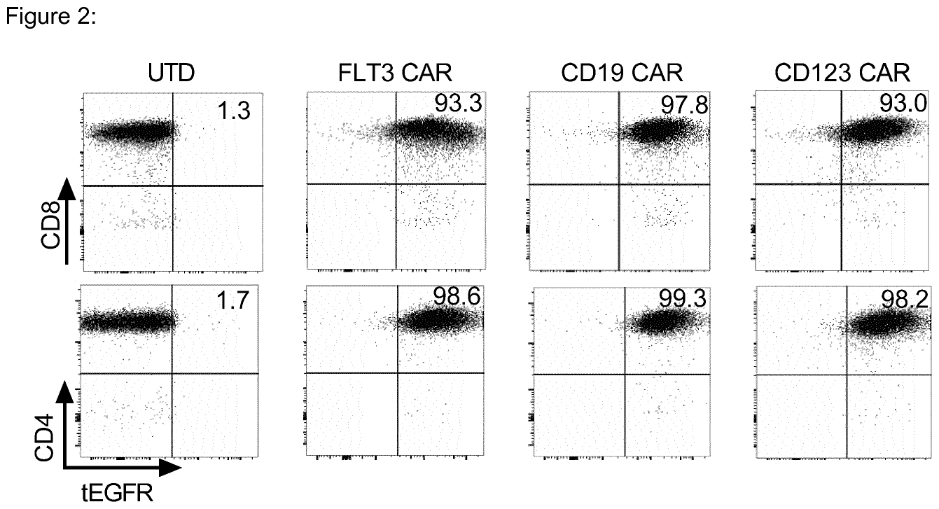

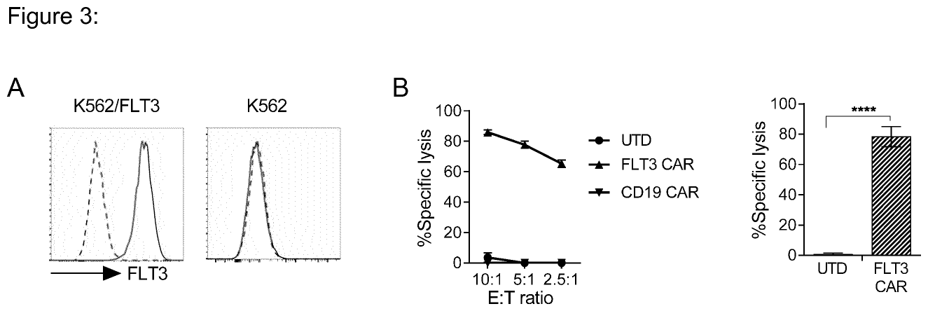

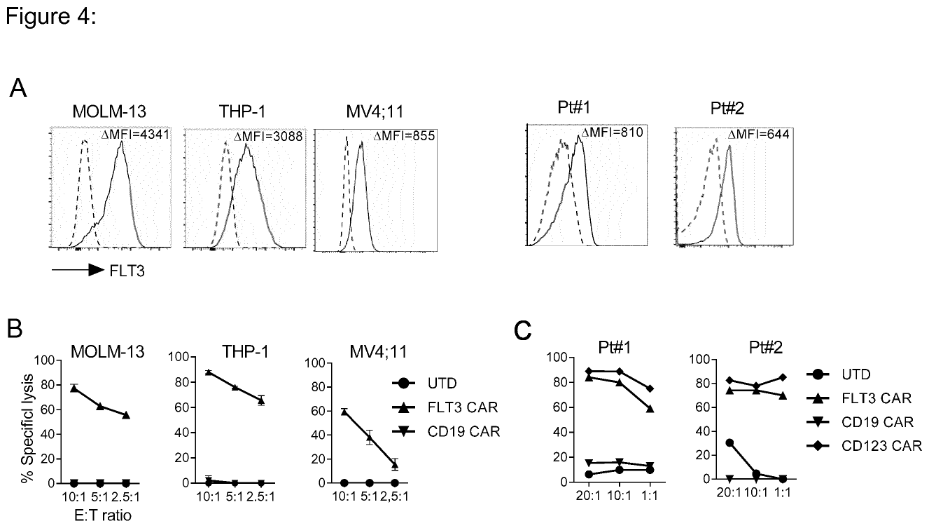

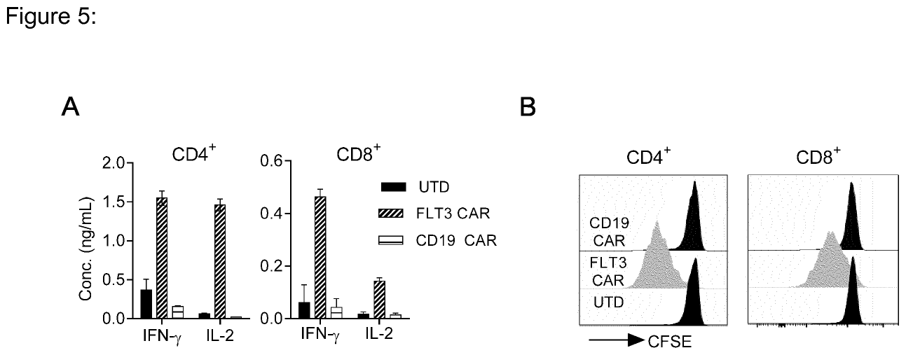

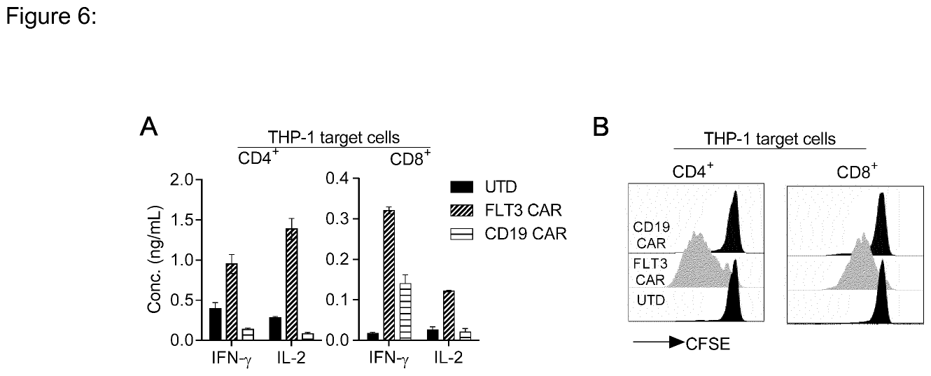

We constructed a CAR transgene comprising a targeting domain derived from the FLT3-specific mAb 4G8 and performed gene-transfer into CD4⁺ and CD8⁺ T cells of healthy donors and AML patients (n=6). FLT3 CAR transduced T cells were enriched to >90% purity using the EGFRt transduction marker prior to expansion and functional testing (FIG. 2). First, we confirmed specific recognition of FLT3 surface protein by CD4⁺ and CD8⁺ FLT3 CAR-T cells using native K562 (phenotype: FLT3⁻) and K562 target cells that had been transduced to stably express wild-type FLT3 (K562/FLT3) (FIG. 3). Then, we included the AML cell lines THP-1 (FLT3 wild-type), MOLM-13 (FLT3-ITD^{+/-}) and MV4;11 (FLT3-ITD^{+/+}) into our analyses and confirmed specific high-level cytolytic activity of CD8⁺ FLT3 CAR-T cells against each of the cell lines at multiple effector to target cell ratios (E:T, range 10:1-2.5:1) (FIG. 4A, B). Further, CD4⁺ and CD8⁺ FLT3 CAR-T cells produced effector cytokines including IFN-γ and IL-2, and underwent productive proliferation after stimulation with each of the AML cell lines, whereas control T cells derived from the same respective donor only showed background reactivity (FIG. 5, 6). Because the FLT3 CAR binds to an epitope in the extracellular domain of FLT3, recognition of AML cells was independent from the mutation status of the intracellular tyrosine kinase domain, but rather correlated with the antigen density of FLT3 surface protein on target cells as assessed by mean fluorescence intensity (MFI) (THP-1˜MOLM-13>MV4;11) (FIG. 4A).

We also confirmed potent activity of patient-derived FLT3 CAR-T cells against FLT3-ITD⁴ primary AML cells, with strong cytolytic activity leading to eradication of >80% AML blasts within as short as 4 hours (E:T, range 20:1-1:1) (FIG. 4A,B). Notably, the antileukemia activity of FLT3 CAR-T cells against primary AML blasts was equivalent to T cells expressing an analogously designed CAR specific for the alternative AML target antigen CD123 (FIG. 4B).

FLT3 CAR-T Cells Induce Durable Remission of AML in a Xenograft Model In Vivo

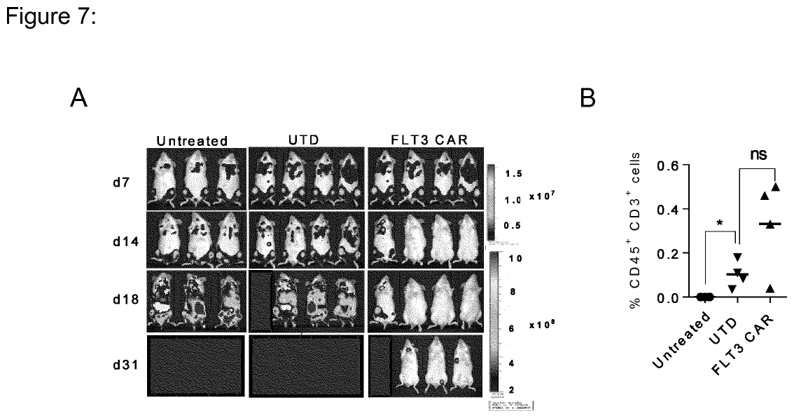

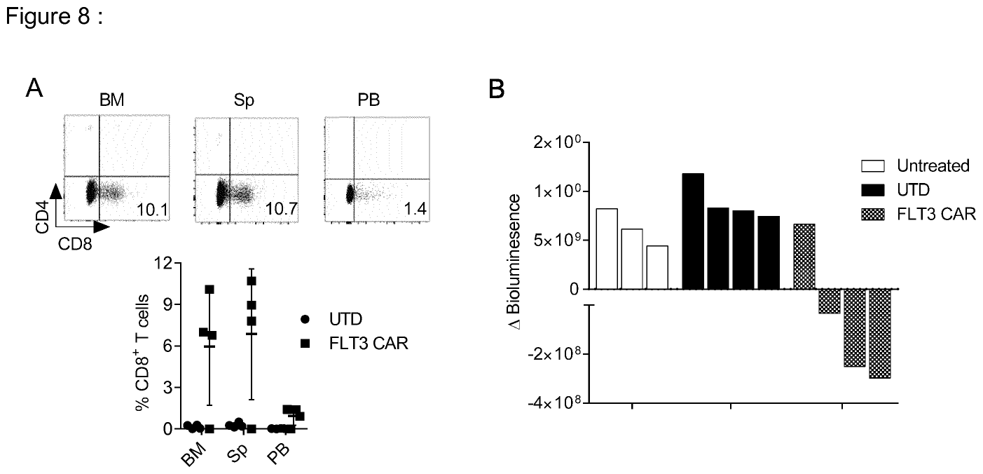

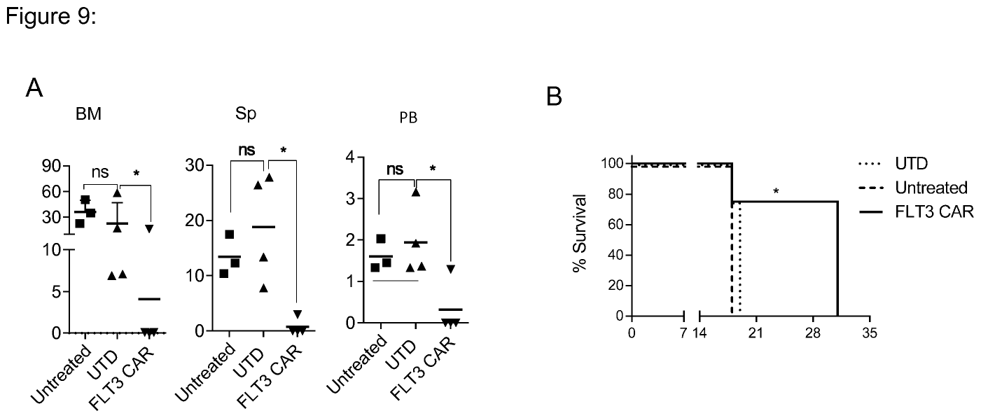

We performed experiments in a xenograft model of AML in immunodeficient NSG mice to analyze the function of FLT3 CAR-T cells in vivo. Following inoculation with ffLuc_GFP-transduced MOLM-13 AML cells, mice rapidly developed systemic leukemia with circulating leukemia cells in peripheral blood, and infiltration of bone marrow and spleen (FIG. 7A). Leukemia-bearing mice were treated with a single dose of 5×10⁶ FLT3 CAR-modified or untransduced T cells, with cell products consisting of equal proportions of CD4⁺ and CD8⁺ T cells, or received no treatment. We observed a strong antileukemia effect in all mice that showed engraftment of FLT3 CAR-T cells. In these mice, FLT3 CAR-T cells increased in number during the antileukemia response, and could readily be detected in peripheral blood at multiple time points; as well as in bone marrow and spleen at the end of the experiment, confirming persistence for >3 weeks after adoptive transfer (FIG. 7B, 8A). Serial bioluminescence imaging confirmed the strong antileukemia activity in all mice with FLT3 CAR-T cell engraftment, whereas mice with CAR-T cell engraftment failure, mice that had been treated with control T cells and untreated mice showed rapid leukemia progression (FIG. 7A, 8B). Further flow cytometric analyses confirmed sustained complete remission of AML cells from bone marrow and spleen (FIG. 9A). Kaplan-Meier analysis showed significantly longer overall survival after treatment with FLT3 CAR-T cells compared with control T cells and no treatment (p<0.05) (FIG. 9B). Of note, in all mice that had responded to FLT3 CAR-T cell therapy, we also observed recurrence of extramedullary late disease, consistent with previous reports of CAR-T cell therapy in NSG mouse models^{37, 43} (FIG. 7A). Expression of FLT3 on AML cells from extramedullary late disease manifestations was detectable at similar levels as on native MOLM-13 cells, i.e. antigen loss had not occurred. In aggregate, our data show that FLT3 CAR-T cells confer potent antileukemia activity against FLT3 wild-type and FLT3-ITD⁺ AML cell lines and primary AML cells in vitro and in vivo.

Midostaurin Induces Increased FLT3 Surface Protein Expression in FLT3-ITD⁺ AML Cells

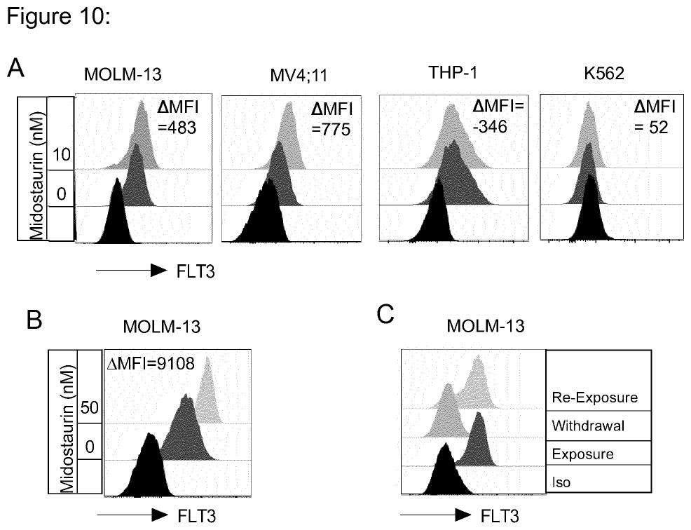

An observation from clinical studies in patients with FLT3-ITD⁺ AML is upregulation of FLT3 as a compensatory mechanism of AML blasts to counteract the effect of FLT3 inhibitors—a mechanism that we hypothesized could be exploited to enhance the antileukemia efficacy of FLT3 CAR-T cells^{24, 25}. We cultured native MOLM-13 AML cells (MOLM-13^{Native}) (FLT3-ITD⁺¹) in the presence of the FLT3 inhibitor midostaurin)(MOLM-13^{mido} using a 10-nM dose. We analyzed FLT3 expression on MOLM-13^{mido} by flow cytometry after 2-3 weeks of exposure to the drug and indeed observed significantly higher levels of FLT3 surface protein as assessed by MFI compared to MOLM-13^{Native} cells (n=2 experiments, p<0.05) (FIG. 10A). Further, we slowly increased midostaurine concentration from 10 nM to 50 nM in next 8-10 weeks and observed further increase in FLT3 expression (FIG. 10B). Interestingly, withdrawal of midostaurin led to a decrease in FLT3 expression on MOLM-13 cells to baseline or slightly below baseline levels within 2 days, but increased again upon re-exposure to the drug (FIG. 10C). After primary exposure to midostaurin, we observed a moderate cytotoxic effect and slower expansion of MOLM-13^{mido} cells compared to MOLM-13^{Native} cells for approx. 2 weeks. However, despite continuous supplementation to the culture medium, the cytotoxic effect of midostaurin subsequently ceased and the expansion of MOLM-13^{mido} cells accelerated, suggesting they had acquired resistance.

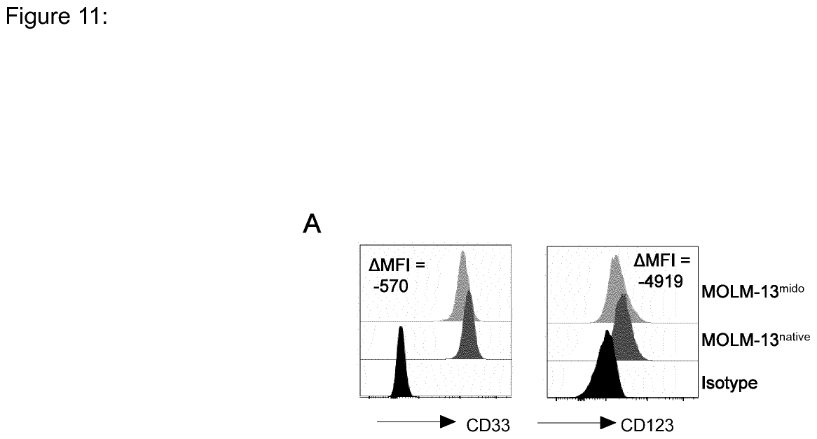

An increase in FLT3 expression upon exposure to midostaurin was also observed with MV4;11 AML cells (FLT3-ITD^{+/+}), but did not occur in several cell lines expressing wild-type FLT3, i.e. THP-1 AML cells, K562 erythro-myeloid leukemia, suggesting upregulation of FLT3 expression in response to midostaurin treatment specifically occurred in FLT3-ITD⁺ AML cells (FIG. 10A,B). In contrast to FLT3, CD33 expression on MOLM-13 was slightly reduced while we observed significant reduction in CD123 expression (FIG. 11).

Higher FLT3 Expression on AML MOLM-13^{mido} Cells Leads to Enhanced Antileukemia Reactivity of FLT3 CAR-T Cells In Vitro

We anticipated that higher expression of FLT3 on MOLM-13^{mido} cells would augment recognition by FLT3 CAR-T cells. Because of the rapid modulation of FLT3 expression upon exposure to and withdrawal of midostaurin, FLT3 CAR-T cells would best be administered concomitantly with the drug to maximize the synergistic antileukemia effect. It is known that TKI may interfere with T-cell signaling and we therefore confirmed that midostaurin per se did not affect function of FLT3 CAR-T cells. Then, we evaluated the antileukemia reactivity of FLT3 CAR-T cells against midostaurin pre-treated MOLM-13^{mido} in the presence of the drug.

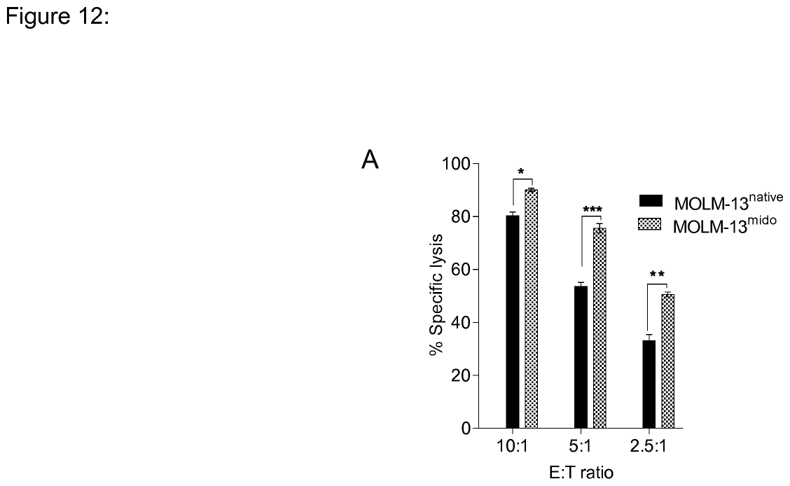

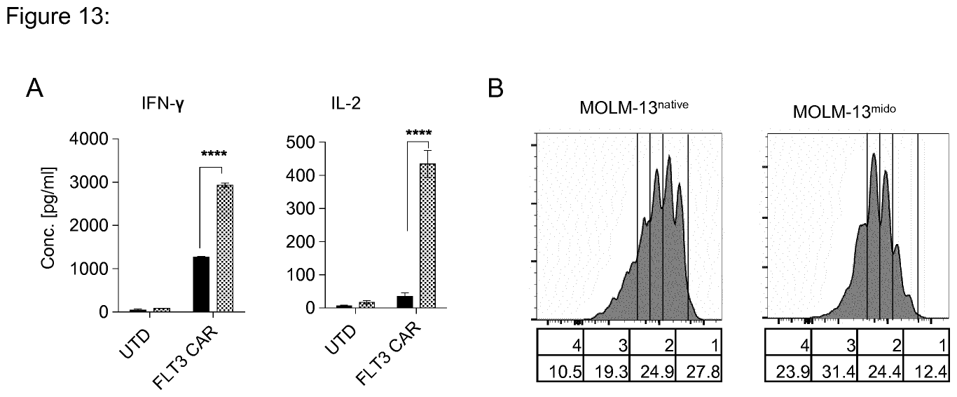

Indeed, we observed significantly higher cytolytic activity of CD8⁺ FLT3 CAR-T cells against MOLM-13^{mido} (90.0±0.9) compared to native MOLM-13^{native} cells (80.3±2.0) at 10:1 E:T ratio (p<0.05) (FIG. 12). Further at comparatively lower E:T ratio, we observed 1.4 fold (75.6±2.5 vs 53.5±2.2 at 5:1 E:T ratio) and 1.5 fold (50.6±1.3 vs 33.0±3.4 at 2.5:1 E:T ratio) increase in cytolytic activity of CD8⁺ FLT3 CAR-T cells (FIG. 12). Next, we analyzed specific cytokine production by FLT3 CAR-T cells against MOLM-13^{mido} compared to native MOLM-13^{native} cells. Indeed, We observed 2.3 fold higher (MOLM-13^{mido} vs MOLM-13^{native}, 2934.0±26.0 vs 1263.0±11.0 pg/mL) IFN-γ production and 12.4 fold higher (MOLM-13^{mido} vs MOLM-13^{native}, 434.0±23.0 vs 35.0±6.0 pg/mL) IL-2 production by FLT3-CAR T cells (FIG. 13A). FLT3 CAR T cells proliferated 1.8 fold (proliferation index) higher against MOLM-13^{mido} (% proliferation, MOLM-13^{mido} vs MOLM-13^{native}, 59.4 vs 31.3) compared to native MOLM-13^{native} cells (FIG. 13B). Percentage of T cells proliferated at least 3 and at least 4 times against MOLM-13^{mido} are 23.9 and 31.4 as compared to 10.5 and 19.3 against MOLM-13^{native} respectively (FIG. 13B), demonstrating a significant gain of function.

Crenolanib Induces Increased FLT3 Surface Protein Expression in FLT3-ITD⁺ AML Cells

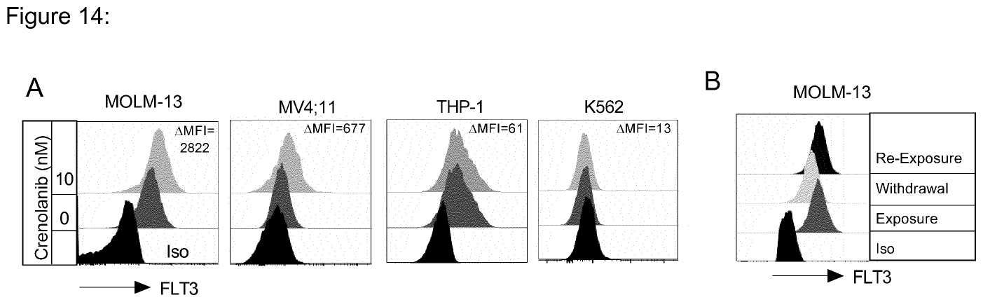

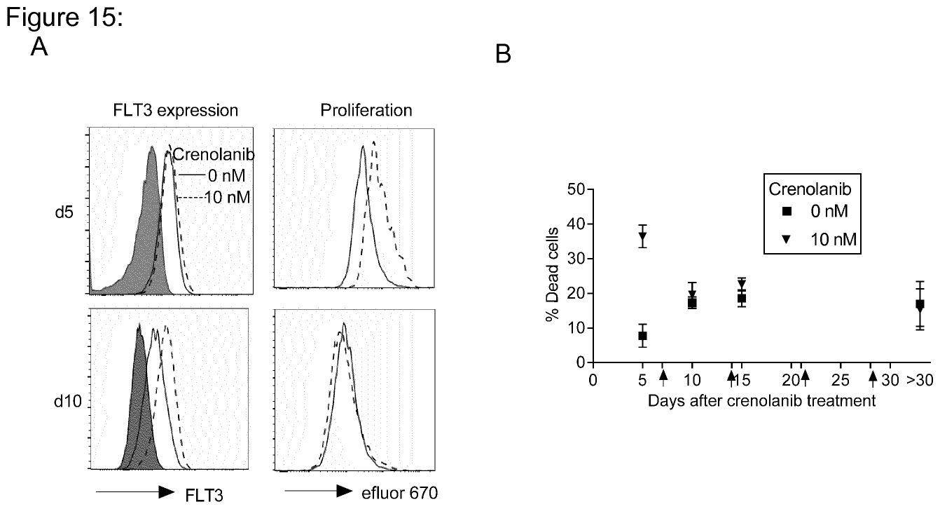

An observation from clinical studies in patients with FLT3-ITD⁺ AML is upregulation of FLT3 as a compensatory mechanism of AML blasts to counteract the effect of FLT3 inhibitors—a mechanism that we hypothesized could be exploited to enhance the antileukemia efficacy of FLT3 CAR-T cells^{24, 25}. We cultured native MOLM-13 AML cells (MOLM-13^{Native}) (FLT3-ITD^{+/-}) in the presence of the FLT3 inhibitor crenolanib (MOLM-13^{Creno}) using a 10-nM dose, which is a clinically achievable serum level^{27, 44}. We analyzed FLT3 expression on MOLM-13^{Creno} by flow cytometry after 5 days of exposure to the drug and indeed observed significantly higher levels of FLT3 surface protein as assessed by MFI compared to MOLM-13^{Native} cells (n=3 experiments, p<0.05) (FIG. 14A). Interestingly, withdrawal of crenolanib led to a decrease in FLT3 expression on MOLM-13 cells to baseline levels within 2 days, but increased again upon re-exposure to the drug (FIG. 14B). After primary exposure to crenolanib, we observed a moderate cytotoxic effect and slower expansion of MOLM-13^{Creno} cells compared to MOLM-13^{Native} cells for approx. 7 days (FIG. 15A,B). However, despite continuous supplementation to the culture medium, the cytotoxic effect of crenolanib subsequently ceased and the expansion of MOLM-13^{Creno} cells accelerated, suggesting they had acquired resistance.

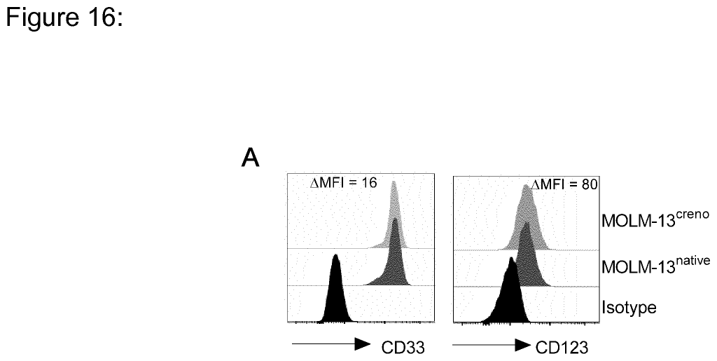

An increase in FLT3 expression upon exposure to crenolanib was also observed with MV4;11 AML cells (FLT3-ITD^{+/+}), but did not occur in several cell lines expressing wild-type FLT3, i.e. THP-1 AML cells, JeKo-1 mantle cell lymphoma, and K562 erythro-myeloid leukemia, suggesting upregulation of FLT3 expression in response to crenolanib treatment specifically occurred in FLT3-ITD⁺ AML cells (FIG. 14A). In contrast to FLT3, CD33 and CD123 expression on both MOLM-13 and MV4;11 was not affected by crenolanib and did not increase (FIG. 16).

Higher FLT3 Expression on Crenolanib-Treated MOLM-13 AML Cells Leads to Enhanced Antileukemia Reactivity of FLT3 CAR-T Cells In Vitro

We sought to analyze whether the higher antigen density of FLT3 on MOLM-13^{Creno} would enhance recognition by FLT3 CAR-T cells. Our earlier data showed rapid modulation of FLT3 expression upon exposure to and withdrawal of crenolanib (FIG. 15B), suggesting maximum reactivity of FLT3 CAR-T cells against MOLM-13^{Creno} would be accomplished in the presence of the drug. It is known that TKI may interfere with T-cell activation and function^{45, 46}, and we therefore confirmed that crenolanib per se did not affect the effector function of FLT3 CAR-T cells.

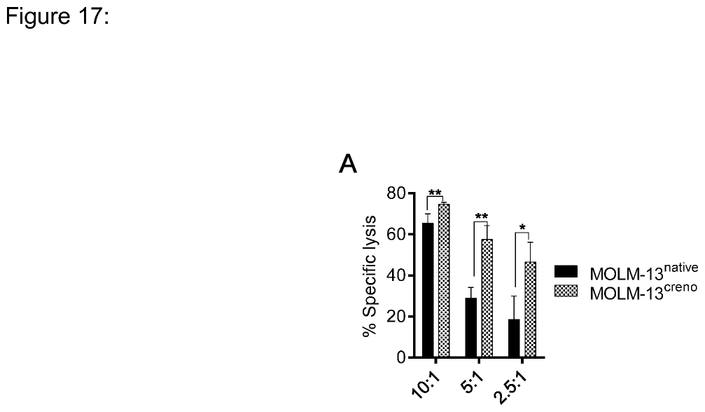

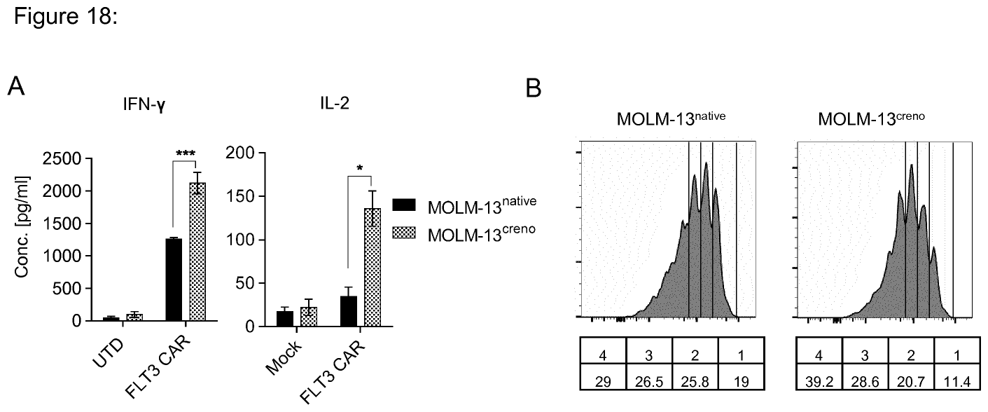

Indeed, we observed superior cytolytic activity of CD8⁺ FLT3 CAR-T cells against MOLM-13^{creno} (74.7±0.8) compared to native MOLM-13^{native} cells (68.0±0.9) at 10:1 E:T ratio (p<0.05) (FIG. 17). Further at comparatively lower E:T ratio, we observed 2 fold (MOLM-13^{creno} vs MOLM-13^{native} 57.5±5.5 vs 28.9±4.2 at 5:1 E:T ratio) and 2.5 fold (46.4±4.9 vs 18.5±9.3 at 2.5:1 E:T ratio) increase in cytolytic activity of CD8⁺ FLT3 CAR-T cells (FIG. 17). Next, we analyzed cytokine production by FLT3 CAR-T cells against MOLM-13^{creno} compared to native MOLM-13^{native} cells. Indeed, we observed 1.4 fold higher (MOLM-13^{creno} vs MOLM-13^{native}, 2121.1±135.1 vs 1523.0±229.8 pg/mL) IFN-γ production and 3.9 fold higher (MOLM-13^{creno} vs MOLM-13^{native}, 135.8±16.5 vs 34.7±8.8 pg/mL) IL-2 production by FLT3-CAR T cells (FIG. 18A). Percentage of T cells proliferated at least 3 and at least 4 times against MOLM-13^{creno} are 39.2 and 28.6 as compared to 29.0 and 26.5 against MOLM-13^{native} respectively (FIG. 18B), demonstrating a significant gain of function.

Quizartinib Induces Increased FLT3 Surface Protein Expression in FLT3-ITD⁺ AML Cells

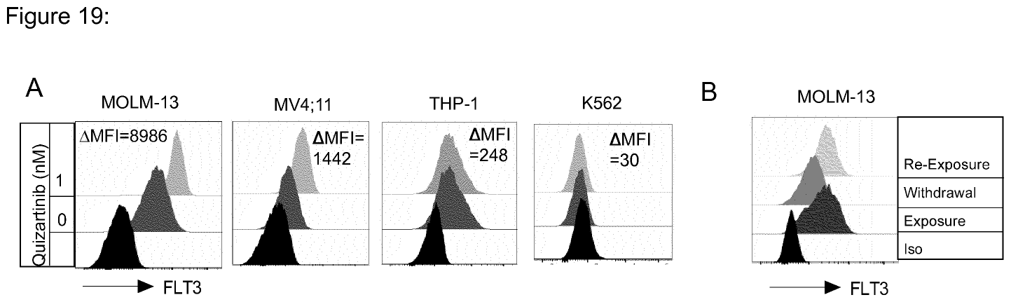

An observation from clinical studies in patients with FLT3-ITD⁺ AML is upregulation of FLT3 as a compensatory mechanism of AML blasts to counteract the effect of FLT3 inhibitors—a mechanism that we hypothesized could be exploited to enhance the antileukemia efficacy of FLT3 CAR-T cells^{24, 25}. We cultured native MOLM-13 AML cells (MOLM-13^{Native}) (FLT3-ITD^{+/-}) in the presence of the FLT3 inhibitor quizartinib (MOLM-13^{Quiza}) using a 1-nM dose, which is a clinically achievable serum level^{27, 44}. We analyzed FLT3 expression on MOLM-13^{Quiza} by flow cytometry after 5 days of exposure to the drug and indeed observed significantly higher levels of FLT3 surface protein as assessed by MFI compared to MOLM-13^{Native} cells (n=3 experiments, p<0.05) (FIG. 19A). Interestingly, withdrawal of quizartinib led to a decrease in FLT3 expression on MOLM-13 cells to baseline levels within 2 days, but increased again upon re-exposure to the drug (FIG. 19B). After primary exposure to quizartinib, we observed a moderate cytotoxic effect and slower expansion of MOLM-13^{Quiza} cells compared to MOLM-13^{Native} cells for approx. 7 days. However, despite continuous supplementation to the culture medium, the cytotoxic effect of quizartinib subsequently ceased and the expansion of MOLM-13^{Quiza} cells accelerated, suggesting they had acquired resistance.

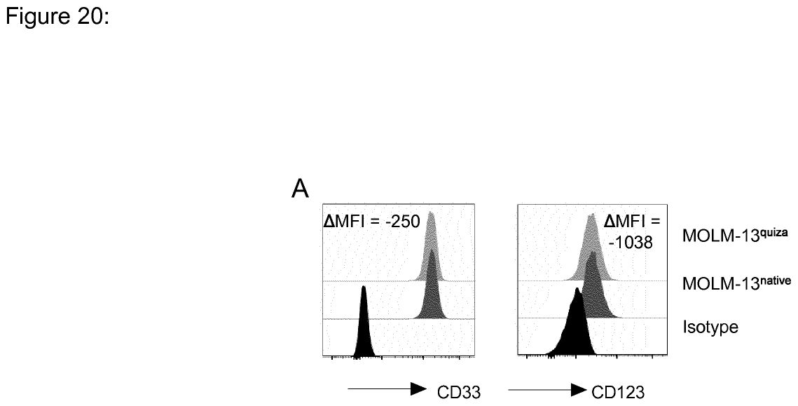

An increase in FLT3 expression upon exposure to quizartinib was also observed with MV4;11 AML cells (FLT3-ITD^{+/+}), but did not occur in several cell lines expressing wild-type FLT3, i.e. THP-1 AML cells, JeKo-1 mantle cell lymphoma, and K562 erythro-myeloid leukemia, suggesting upregulation of FLT3 expression in response to quizartinib treatment specifically occurred in FLT3-ITD⁺ AML cells (FIG. 19B). In contrast to FLT3, CD33 and CD123 expression on both MOLM-13 and MV4;11 was not affected by quizartinib and did not increase (FIG. 20).

Higher FLT3 Expression on AML MOLM-13^{quiza} Cells Leads to Enhanced Antileukemia Reactivity of FLT3 CAR-T Cells In Vitro

We anticipated that higher expression of FLT3 on MOLM-13^{quiza} cells would augment recognition by FLT3 CAR-T cells. Because of the rapid modulation of FLT3 expression upon exposure to and withdrawal of quizartinib, FLT3 CAR-T cells would best be administered concomitantly with the drug to maximize the synergistic antileukemia effect. Then, we evaluated the antileukemia reactivity of FLT3 CAR-T cells against quizartinib pre-treated MOLM-13^{quiza} in the presence of the drug.

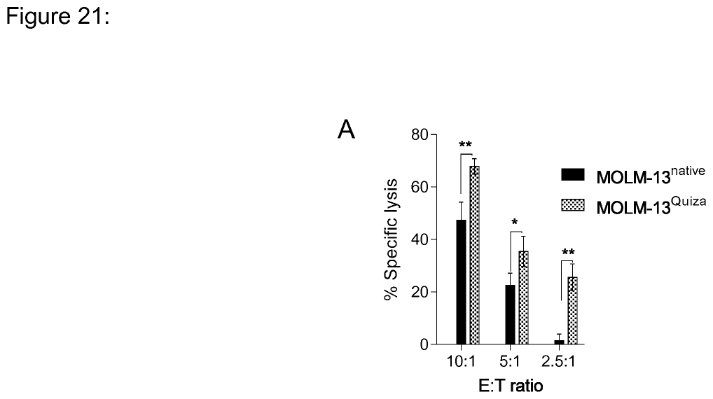

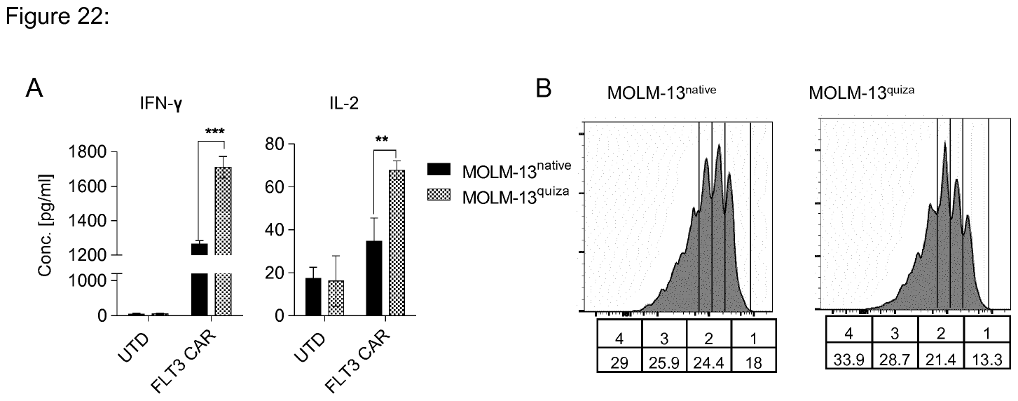

Indeed, we observed superior cytolytic activity of CD8⁺ FLT3 CAR-T cells against MOLM-13^{quiza} (67.9±2.4) compared to native MOLM-13^{native} cells (47.3±5.6) at 10:1 E:T ratio (p<0.05) (FIG. 21). Further at comparatively lower E:T ratio, we observed 1.6 fold (MOLM-13^{quiza} vs MOLM-13^{native} 35.5±4.7 vs 22.5±3.3 at 5:1 E:T ratio) and 17.7 fold (25.6±4.1 vs 1.4±2.0 at 2.5:1 E:T ratio) increase in cytolytic activity of CD8⁺ FLT3 CAR-T cells (FIG. 21). Next, we analyzed cytokine production by FLT3 CAR-T cells against MOLM-13^{quiza} compared to native MOLM-13^{native} cells. Indeed, We observed 1.4 fold higher (MOLM-13^{quiza} vs MOLM-13^{native}, 1711.0±36.0 vs 1263.1±11.0 pg/mL) IFN-γ production and 1.9 fold higher (MOLM-13^{quiza} vs MOLM-13^{native}, 68.0±3.0 vs 35.0±6.0 pg/mL) IL-2 production by FLT3-CAR T cells (FIG. 22A). Percentage of T cells proliferated at least 3 and at least 4 times against MOLM-13^{quiza} are 33.9 and 28.7 as compared to 29.0 and 25.9 against MOLM-13^{native} respectively (FIG. 22B), demonstrating a significant gain of function.

FLT3 CAR-T Cells and the FLT3 Inhibitor Crenolanib Act Synergistically in Mediating Regression of AML In Vivo

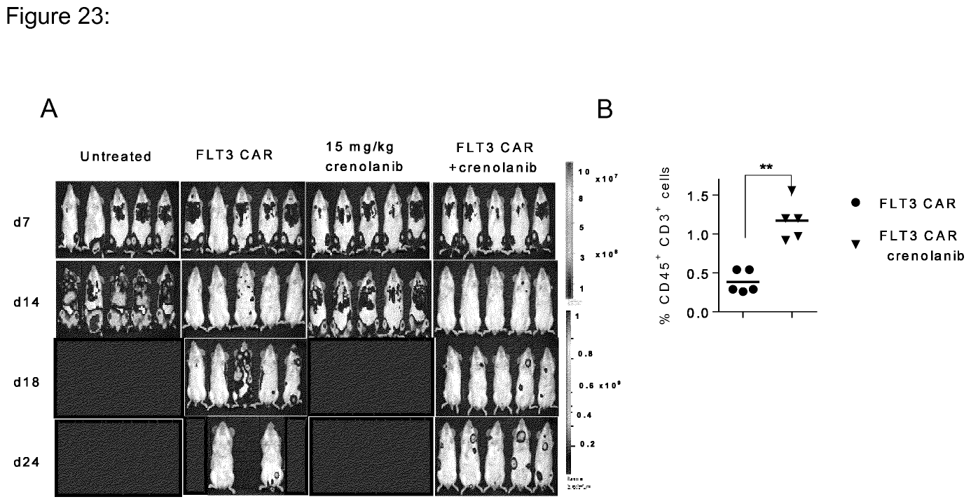

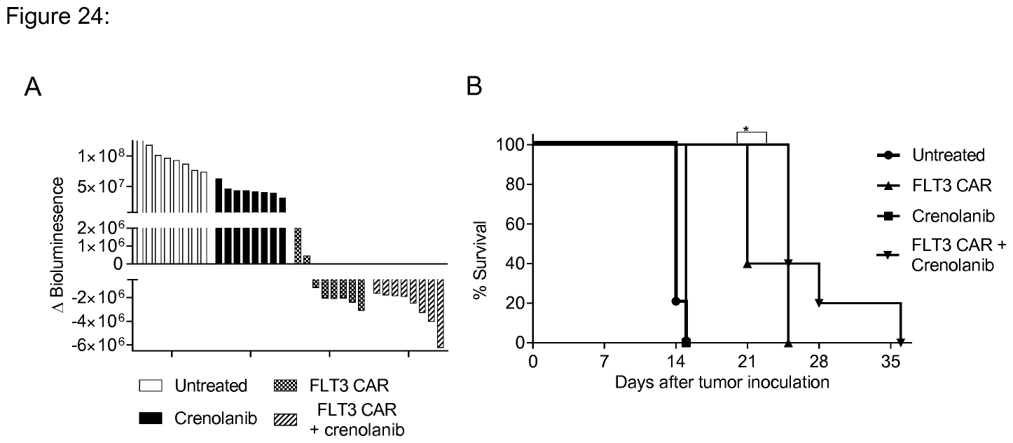

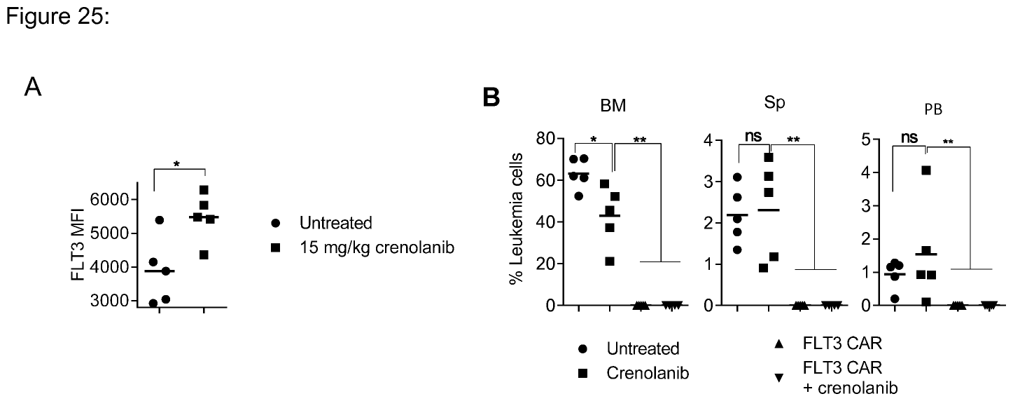

This encouraged us to examine the antileukemia effect of FLT3 CAR-T cells in combination with crenolanib in the MOLM-13/NSG xenograft model. Mice were inoculated with MOLM-13^{native} AML cells on day 0 and treated on day 7 with either FLT3 CAR-T cells alone, crenolanib alone (15 mg/kg body weight as i.p. injection qd), the combination treatment with FLT3 CAR-T cells and crenolanib, or left untreated. We observed potent antileukemia efficacy in mice receiving the combination treatment with FLT3 CAR-T cells and crenolanib (FIG. 23A). There was superior engraftment and in vivo expansion of FLT3 CAR-T cells by flow cytometry (FIG. 23B), a higher overall response rate (combination: n=8/8, 100% vs. FLT3 CAR-T cells mono n=6/8, 75% vs. crenolanib mono n=0/8, 0% vs. no treatment n=0/0, 0%), faster and deeper remissions as assessed by bioluminescence imaging (FIG. 23A, 24A), as well as improved overall survival of mice receiving the FLT3 CAR-T cell and crenolanib combination, compared to monotherapy with FLT3 CAR-T cells and crenolanib, and no treatment, respectively (p<0.05) (FIG. 24B). Crenolanib monotherapy had only a minute antileukemia effect and MOLM-13 cells recovered from peripheral blood and bone marrow at the experiment endpoint had uniformly and strongly upregulated FLT3, consistent with our earlier observation in vitro (FIG. 25A). Also with the combination treatment, mice experienced delayed extramedullary late disease. At the experiment endpoint, peripheral blood, bone marrow and spleen in mice treated with the FLT3 CAR-T cell/crenolanib combination and FLT3 CAR-T cells monotherapy were free from AML cells, whereas mice receiving crenolanib monotherapy and untreated mice showed a high degree of leukemia infiltration (FIG. 25B). Collectively, the data show that FLT3 CAR-T cells and crenolanib can be used synergistically in combination therapy to confer a potent antileukemia effect against FLT3-ITD+AML cells in vitro and in vivo.

### Example 2

Materials and Methods:

Human Subjects

Peripheral blood was obtained from healthy donors and adult AML patients after written informed consent to participate in research protocols approved by the Institutional Review Board of the participating institutions.

Primary AML Cells

Primary AML cells were maintained in RPMI-1640 supplemented with 10% human serum, 2 mM glutamine, 100 U/mL penicillin/streptomycin, and a cytokine cocktail including IL-4 (1000 IU/mL), granulocyte macrophage colony-stimulating factor (GM-CSF) (10 ng/mL), stem cell factor (5 ng/mL) and tumor necrosis factor (TNF)-α (10 ng/mL).

Tumor Cell Lines

The human leukemia cell lines MOLM-13 (ACC 554), THP-1 (ACC 16), MV4;11 (ACC 102), and K562 (ACC 10) were purchased from DSMZ (Deutsche Sammlung von Mikroorganismen and Zellkulturen, Braunschweig, Germany) and cultured in RPMI-1640 supplemented with 10% fetal calf serum (FCS), 2 mM glutamine and 100 U/mL penicillin/streptomycin. All cell lines were transduced with a lentiviral vector encoding a firefly luciferase (ffluc)_green fluorescent protein (GFP) transgene to enable detection by flow cytometry (GFP) and bioluminescence imaging (ffLuc) in mice, and bioluminescence-based cytotoxicity assays. K562/FLT3 was generated by retroviral transduction with the full-length human FLT3 gene.

Flow Cytometric Analysis of FLT3 Expression

Cell surface expression of FLT3 (CD135) was analyzed using a conjugated mouse-anti-human-FLT3 mAb (clone 4G8, BD Pharmagin, BD Biosciences, Germany) and mouse IgG1 isotype control (BD Pharmagin). In brief, 1×10⁶ cells were washed, resuspended in 100 μL PBS/0.5% fetal calf serum and stained with 5 μL of anti-FLT3 mAb or isotype for 30 minutes at 4° C.

CAR Construction

A codon optimized targeting domain comprising the V_{H} and V_{L} segments of the FLT3-specific BV10 mAb¹² was synthesized (GeneArt, ThermoFisher, Regensburg, Germany) and fused to a CAR backbone comprising a short IgG4-Fc Hinge spacer, a CD28 transmembrane and costimulatory moiety and CD3z, in-frame with a T2A element and EGFRt transduction marker (FIG. 1)³²⁻³⁴. The entire transgene was encoded in a lentiviral vector epHIV7 and expressed under control of an EF1/HTLV hybrid promotor^{34, 35}. Similarly, targeting domains specific for CD19 (clone FMC63) and CD123 (clone 32716) were used to generate CD19 and CD123 CARs, respectively^{32, 33, 36, 37}.

Preparation of CAR-Modified T Cells

Lentiviral gene-transfer was performed into CD3/28-bead (ThermoFisher) activated CD4⁺ and CD8⁺ T cells on day 1 after bead stimulation at a moiety of infection (MOI) of 5. T cells were cultured in RPMI-1640 supplemented with 10% human serum, glutamine, 2 mM glutamine, 100 U/mL penicillin/streptomycin and 50 U/mL recombinant human interleukin (IL)-2 (Proleukine, Novartis, Basel, Switzerland)³². CAR-transduced T cells were enriched using biotinylated anti-EGFR mAb (ImClone Systems Inc.) and anti-biotin beads (Miltenyi), prior to expansion using a rapid expansion protocol³⁸ or—for CD19 CAR-T cells—using antigen-specific stimulation with irradiated (80 Gy) CD19⁺ feeder cells³⁸.

Flow Cytometric Analyses of T Cells

Primary AML and peripheral blood mononuclear cells (PBMCs) were stained with 1 or more of the following conjugated mAbs: CD3, CD19, CD34, CD38, CD33, CD45, CD123, CD135 and matched isotype controls (Miltenyi, Bergisch-Gladbach, Germany/BD, Heidelberg, Germany/Biolegend, London, UK). CAR-modified and untransduced T cells were stained with 1 or more of the following conjugated mAbs: CD4, CD8, CD45RA, CD45RO, CD62L, and 7-AAD for live/dead cell discrimination (Miltenyi/BD/Biolegend). CAR-transduced (i.e. EGFRt⁺) T-cells were detected by staining with anti-EGFR antibody that had been biotinylated in-house (EZ-Link™Sulfo-NHS-SS-Biotin, Thermofisher Scientific, IL, according to the manufacturer's instructions) and streptavidin-PE. Flow analyses were done on a FACSCanto (BD) and data analyzed using FlowJo software v9.0.2 (Treestar, Ashland, Oreg.).

Analysis of CAR-T Cell Function In Vitro

Functional analyses were performed as previously described^{32, 33, 39-41}. In brief, target cells expressing firefly luciferase (ffLuc) were incubated in triplicate at 5×10³ cells/well with effector T-cells at various effector to target (E:T) ratios. After 4-hour incubation, luciferin substrate was added to the co-culture and the decrease in luminescence signal in wells that contained target cells and T-cells was measured using a luminometer (Tecan, Männedorf, Switzerland) and compared to target cells alone. Specific lysis was calculated using the standard formula⁴². For analysis of cytokine secretion, 50×10³ T-cells were plated in triplicate wells with target cells at a ratio of 2:1 and IFN-γ and IL-2 production measured by ELISA (Biolegend) in supernatant removed after 24-hour incubation. For analysis of proliferation, 50×10³ T-cells were labeled with 0.2 μM carboxyfluorescein succinimidyl ester (CFSE, ThermoFisher), washed and plated in triplicate wells with target cells at a ratio of 2:1 in medium without exogenous cytokines. After 72-hour incubation, cells were labeled with anti-CD8/CD4 mAb and 7-AAD to exclude dead cells from analysis. Samples were analyzed by flow cytometry and division of live T-cells assessed by CFSE dilution. The cytolytic activity of CAR-modified and control T cells against primary AML cells was analyzed in a FACS-based cytoxicity assay. T cells and AML cells were seeded into 96-well plates at effector:target (E:T) ratios ranging from 20:1 to 1:1, with 10×10³ target cells per well. After 4-24 hours, the cultures were aspirated, stained with 7-AAD to discriminate live and dead cells and anti-CD3/anti-CD33/anti-CD45 mAbs to distinguish T cells and AML cells. To quantitate the number of residual life AML cells, 123-counting beads (e-bioscience, San Diego, Calif.) were used according to the manufacturer's instructions. Flow analyses were done on a FACS Canto II (BD) and data analyzed using Flowio software (Treestar).

In Vivo Experiments

All experiments were approved by the Institutional Animal Care and Use Committees of the participating institutions. NOD.Cg-Prkdc^{scid} Il2rg^{tm1WjI}/SzJ (NSG) mice (female, 6-8 week old) were purchased from Charles River or bred in-house. Mice were inoculated with 1×10⁶ ffluc_GFP⁺MOLM-13 AML cells by tail vein injection on day 0, and received a single dose of 5×10⁶ T cells (in 200 μL of PBS/0.5% FCS) by tail vein injection on day 7. Crenolanib [15 mg/kg; 200 μL of 30% glycerol formal (Sigma Aldrich, Munich, Germany)] was administered intraperitoneally (i.p.) Monday-Friday for 3 consecutive weeks. AML progression/regression was assessed by serial bioluminescence imaging following i.p. administration of D-luciferin substrate (0.3 mg/g body weight) (Biosynth, Staad, Switzerland) using an IVIS Lumina imaging system (Perkin Elmer, Waltham, Mass.). Data was analyzed using Living Image software (Perkin Elmer).

FLT3 Inhibitor Treatment of MOLM-13 AML Cells

MOLM-13 were maintained in RPMI-1640 medium, supplemented with 10% fetal calf serum, 2 mM glutamine, 100 U/mL penicillin/streptomycin, and 10 nM crenolanib or 1 nM quizartinib or 10 nM midostaurin. A complete medium change was performed every 7 days, MOLM-13 cells adjusted to 1×10⁶/mL medium and 2 mL of this cell suspension plated per well in 48-well plates (Costar, Corning, NJ). After 2-3 weeks of culture with 10 nM midostaurine, MOLM-13 cells were exposed to exponentially increasing concentration of midostaurine for next 8-10 weeks to reach 50 nM midostaurin.

Pharmaceutical Drugs and Reagents

Crenolanib, quizartinib (SelleckChemicals, Houston, Tex.), midostaurin (Novartis, Basel, Switzerland/SelleckChemicals, Houston, Tex./Sigma-Aldrich, Steinheim, Germany) were reconstituted in dimethylsulfoxide (DMSO) prior to dilution in medium or 30% glycerol formal (Sigma Aldrich, Munich, Germany) and use in the in vitro or in vivo experiments, respectively.

Statistical Analyses

Statistical analyses were performed using Prism software v6.07 (GraphPad). Unpaired Student's t-tests were used for analysis of data obtained in in vitro experiments. Log-rank (Mantel-Cox) testing was performed to analyze differences in survival observed in in vivo experiments. Differences with a p value <0.05 were considered statistically significant.

Results:

FLT3 CAR-T Cells Eliminate FLT3 Wild-Type and FLT3-ITD+AML Cells

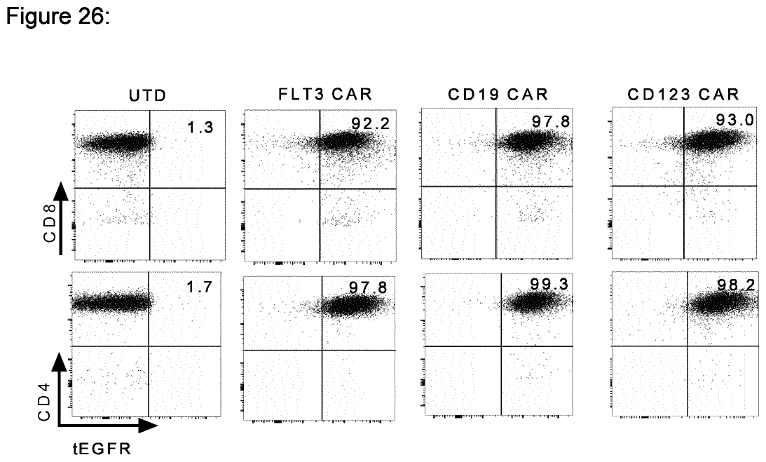

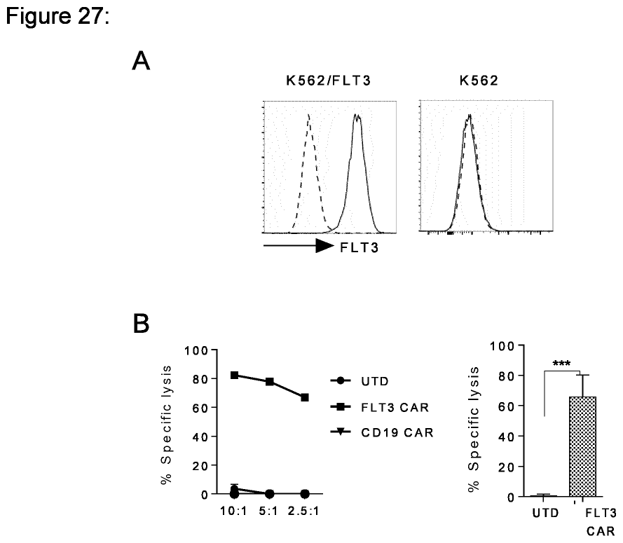

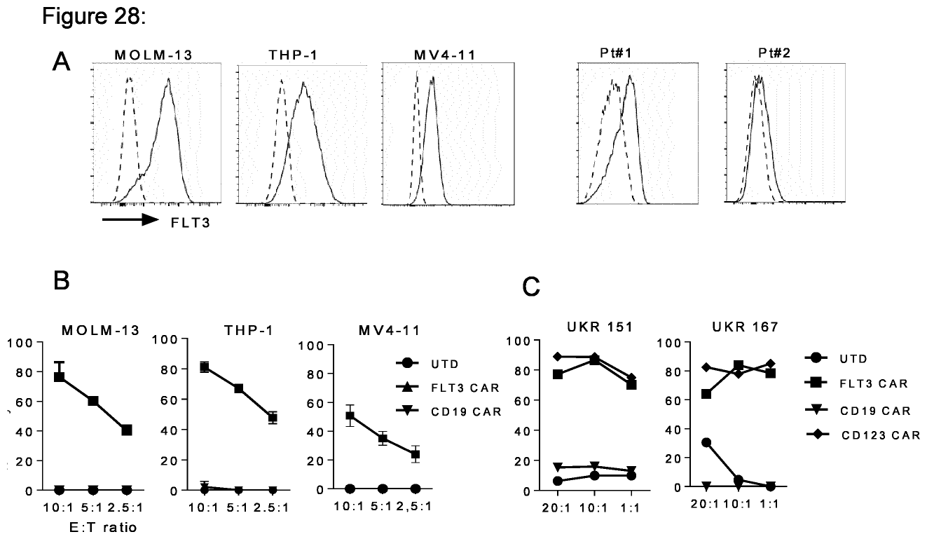

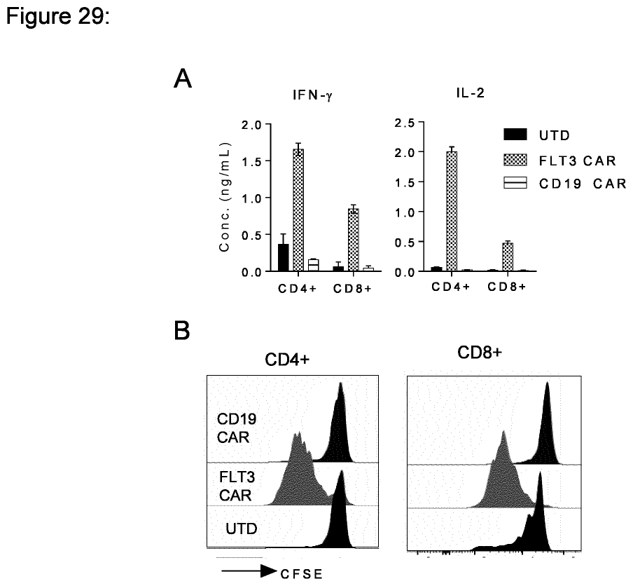

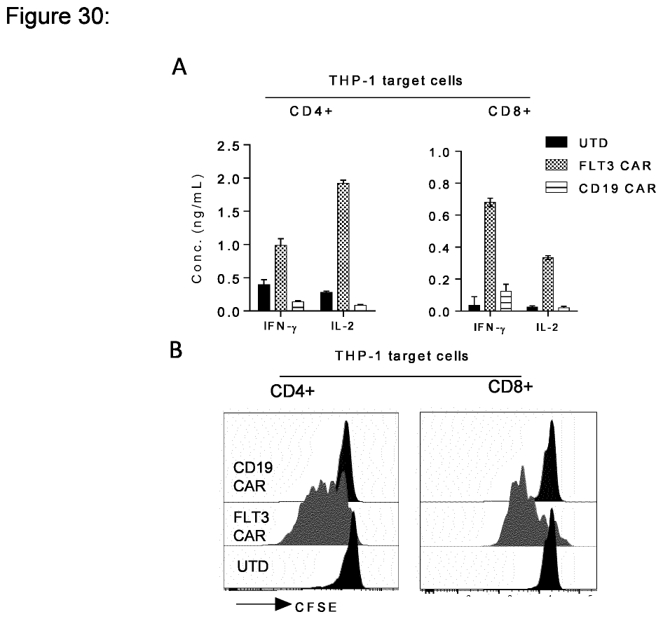

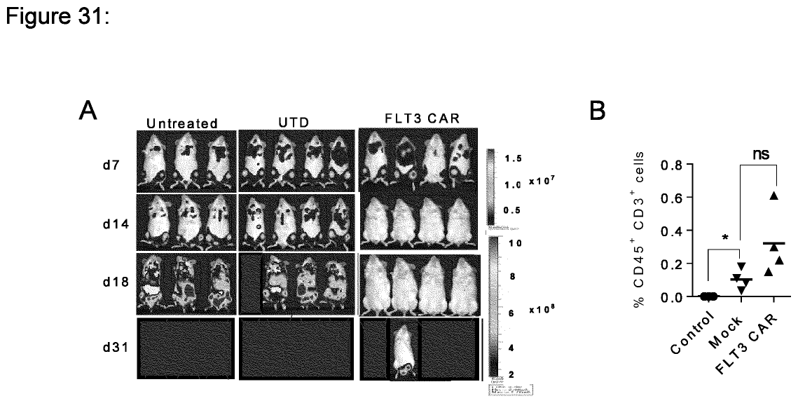

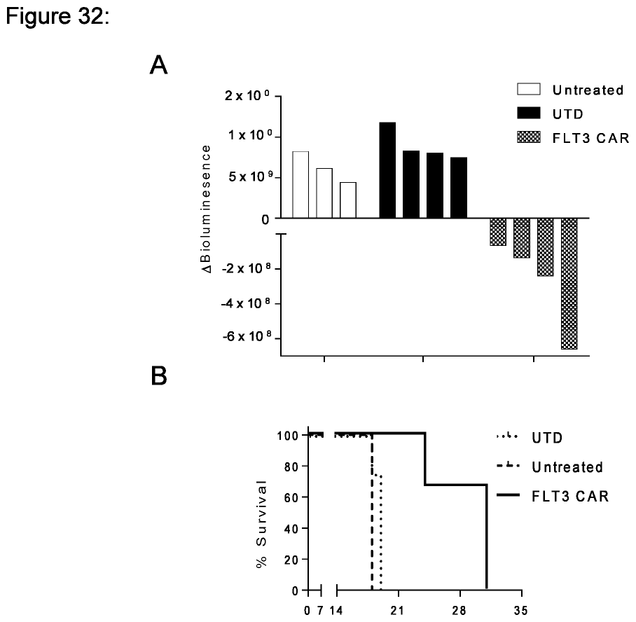

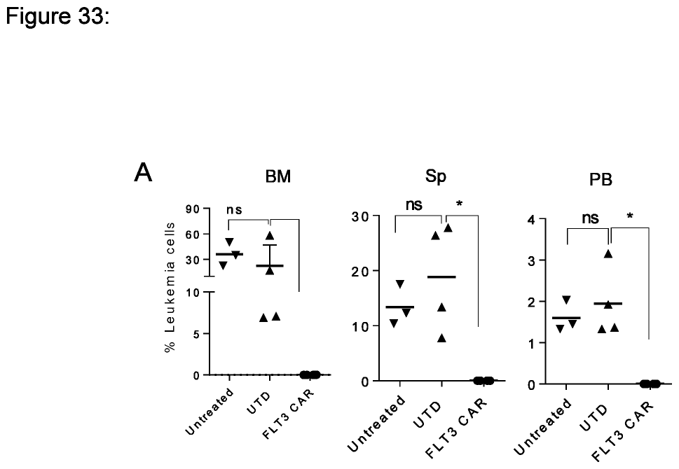

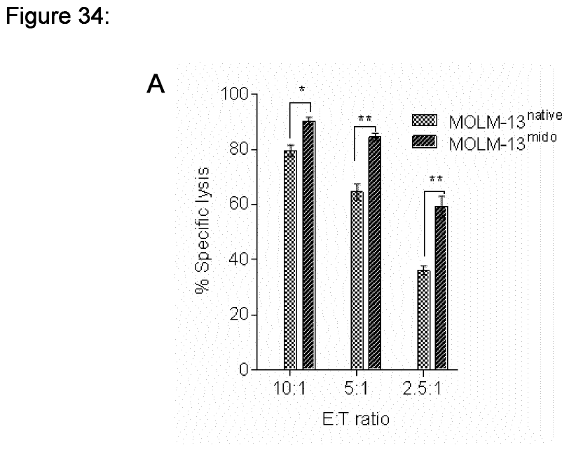

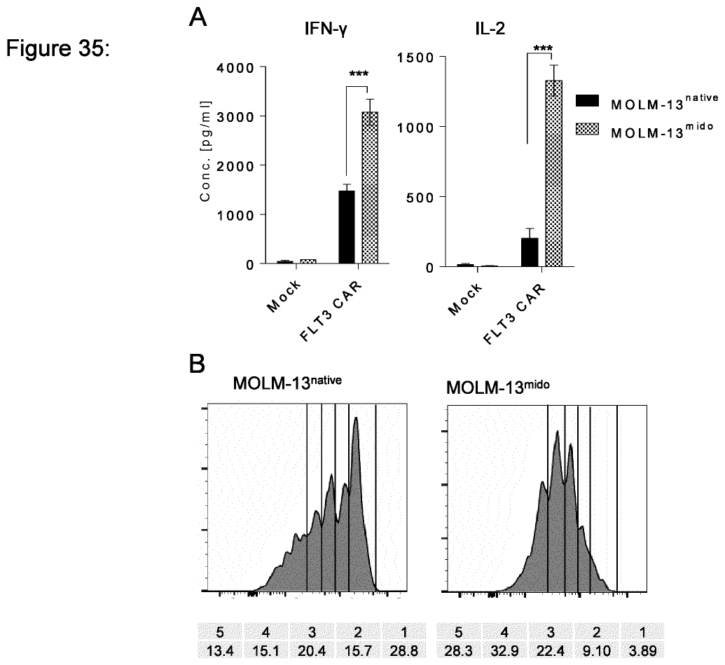

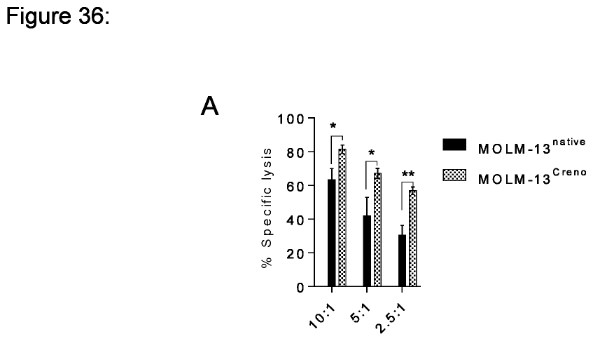

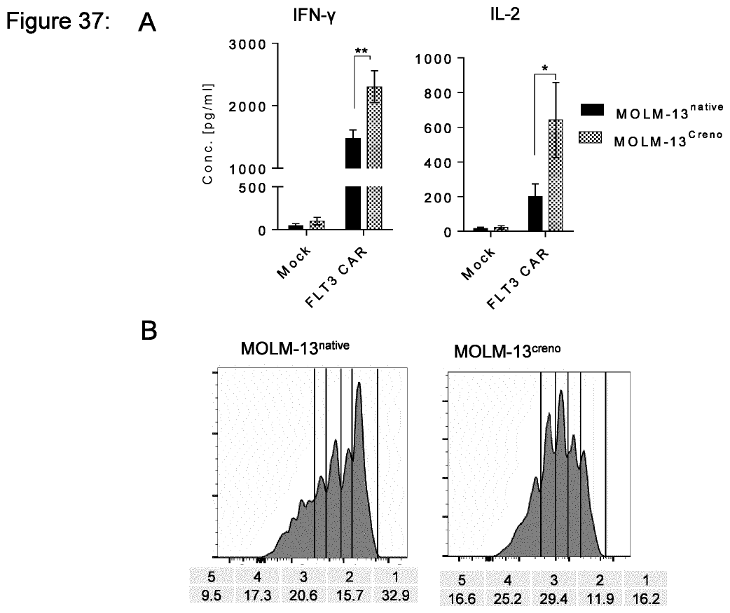

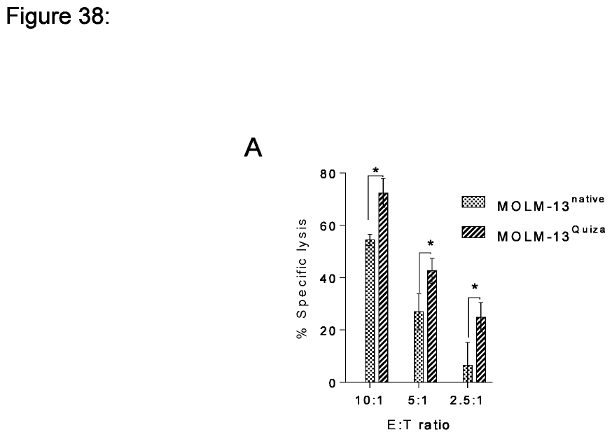

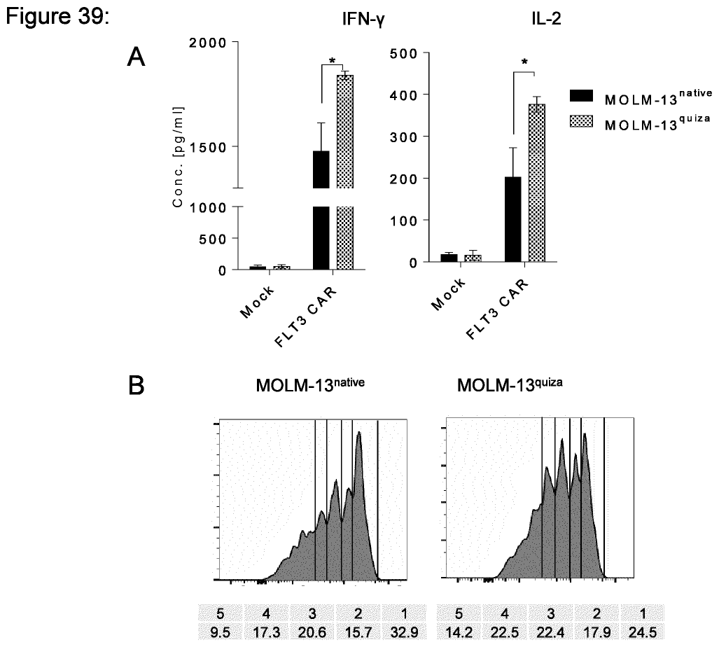

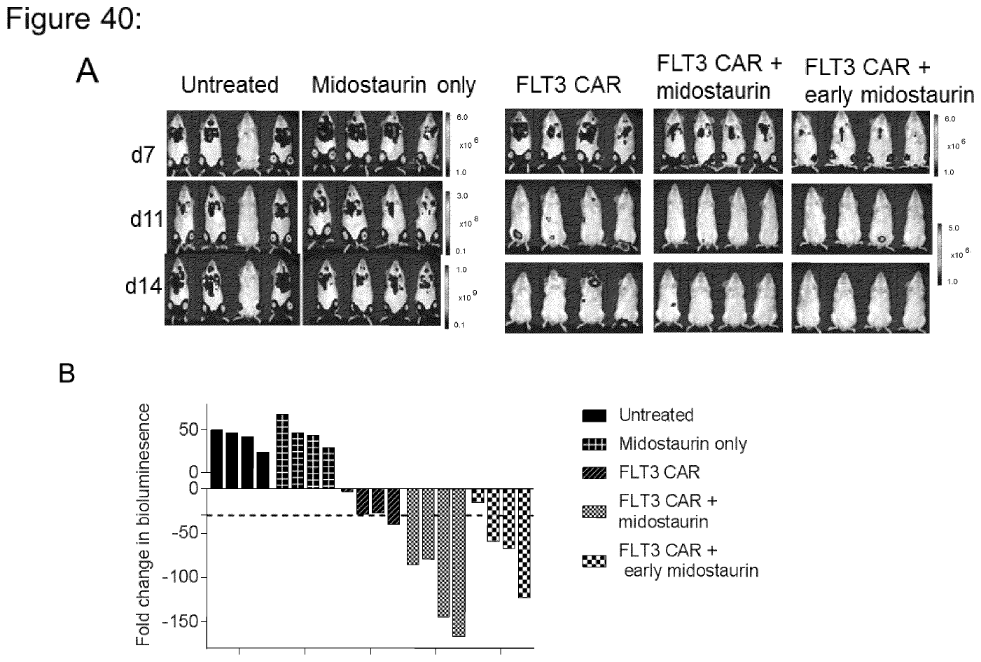

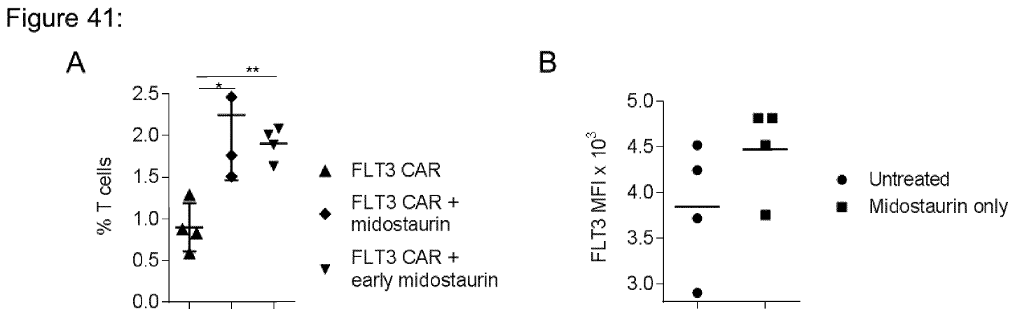

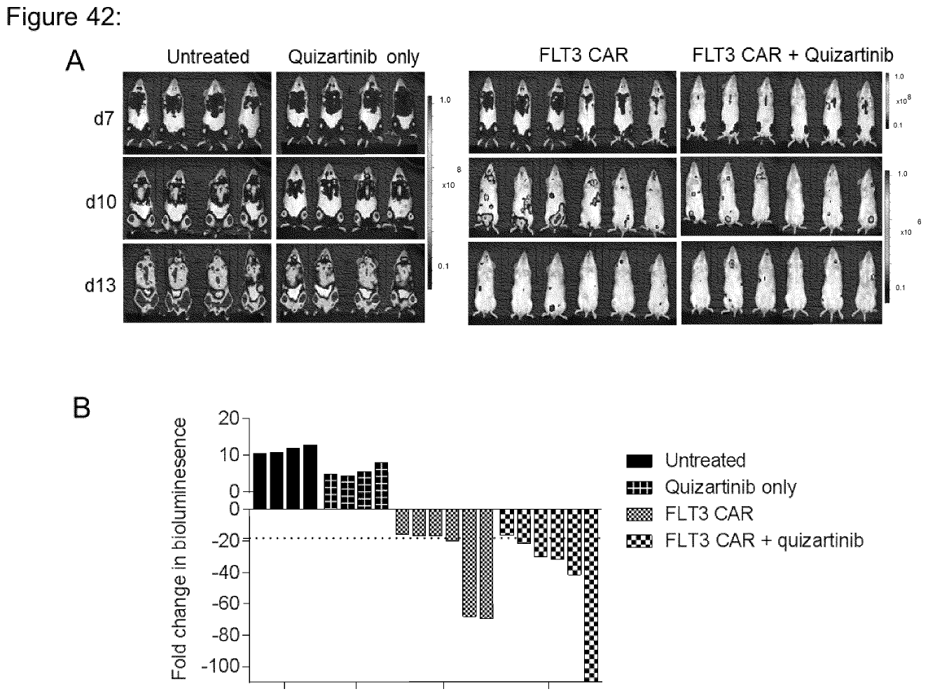

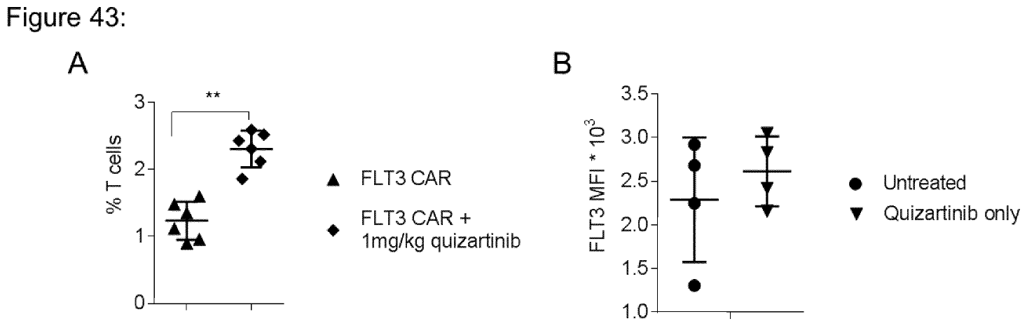

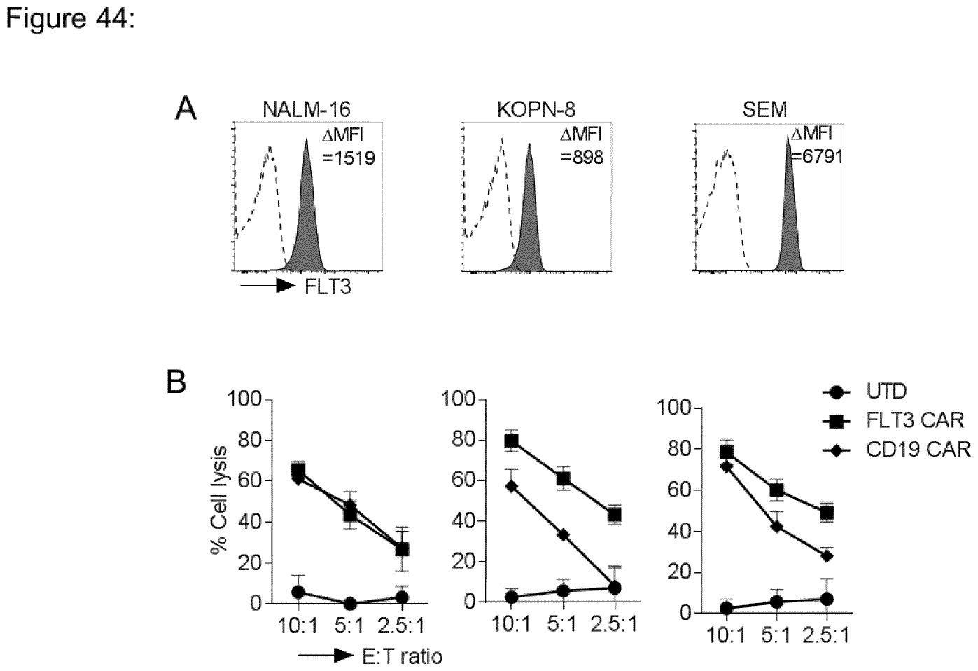

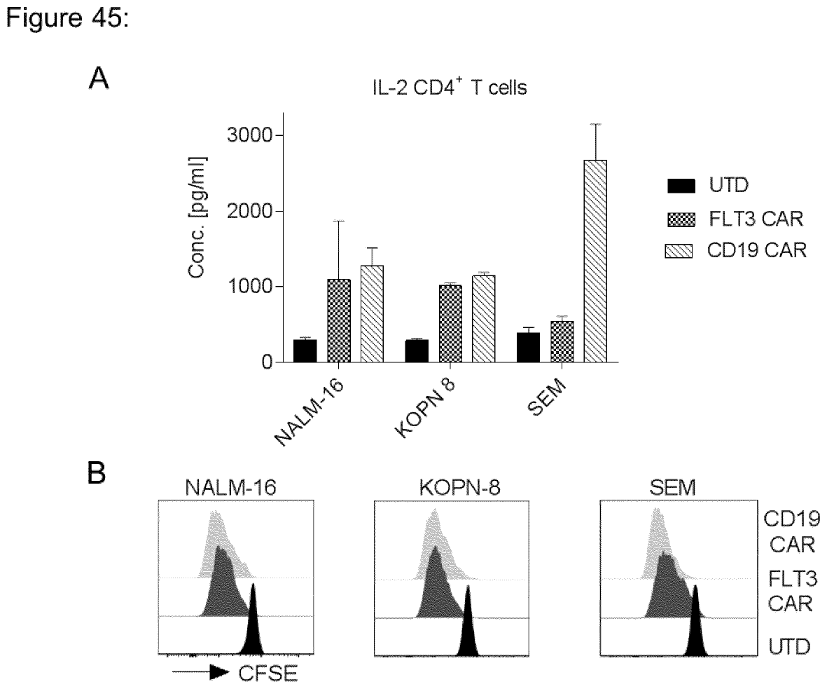

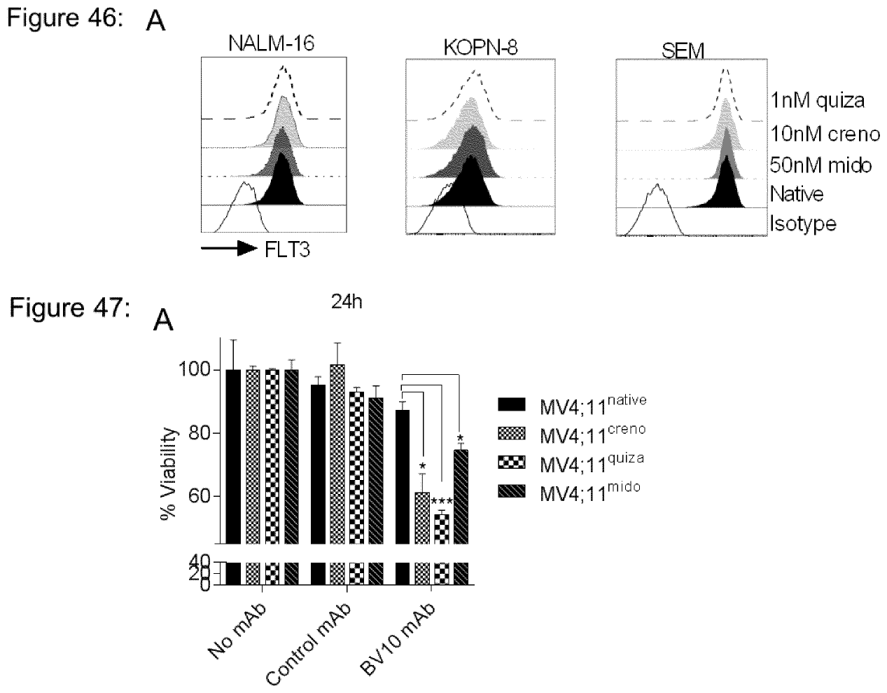

We constructed a CAR transgene comprising a targeting domain derived from the FLT3-specific mAb BV10 and performed gene-transfer into CD4+ and CD8+ T cells of healthy donors and AML patients (n=6). FLT3 CAR transduced T cells were enriched to >90% purity using the EGFRt transduction marker prior to expansion and functional testing (FIG. 26). First, we confirmed specific recognition of FLT3 surface protein by CD4+ and CD8+FLT3 CAR-T cells using native K562 (phenotype: FLT3−) and K562 target cells that had been transduced to stably express wild-type FLT3 (K562/FLT3) (FIG. 27). Then, we included the AML cell lines THP-1 (FLT3 wild-type), MOLM-13 (FLT3-ITD+/−) and MV4;11 (FLT3-ITD+/+) into our analyses and confirmed specific high-level cytolytic activity of CD8+FLT3 CAR-T cells against each of the cell lines at multiple effector to target cell ratios (E:T, range 10:1-2.5:1) (FIG. 28A, B). Further, CD4+ and CD8+FLT3 CAR-T cells produced effector cytokines including IFN-γ and IL-2, and underwent productive proliferation after stimulation with each of the AML cell lines, whereas control T cells derived from the same respective donor only showed background reactivity (FIG. 29, 30). Because the FLT3 CAR binds to an epitope in the extracellular domain of FLT3, recognition of AML cells was independent from the mutation status of the intracellular tyrosine kinase domain, but rather correlated with the antigen density of FLT3 surface protein on target cells as assessed by mean fluorescence intensity (MFI) (THP-1 MOLM-13>MV4;11) (FIG. 28A).

We also confirmed potent activity of patient-derived FLT3 CAR-T cells against FLT3-ITD+ primary AML cells, with strong cytolytic activity leading to eradication of >80% AML blasts within as short as 4 hours (E:T, range 20:1-1:1) (FIG. 28A,B). Notably, the antileukemia activity of FLT3 CAR-T cells against primary AML blasts was equivalent to T cells expressing an analogously designed CAR specific for the alternative AML target antigen CD123 (FIG. 28B).

FLT3 CAR-T Cells Induce Durable Remission of AML in a Xenograft Model In Vivo

We performed experiments in a xenograft model of AML in immunodeficient NSG mice to analyze the function of FLT3 CAR-T cells in vivo. Following inoculation with ffLuc_GFP-transduced MOLM-13 AML cells, mice rapidly developed systemic leukemia with circulating leukemia cells in peripheral blood, and infiltration of bone marrow and spleen (FIG. 31A). Leukemia-bearing mice were treated with a single dose of 5×10⁶ FLT3 CAR-modified or untransduced T cells, with cell products consisting of equal proportions of CD4⁺ and CD8⁺ T cells, or received no treatment. We observed a strong antileukemia effect in all mice that showed engraftment of FLT3 CAR-T cells. In these mice, FLT3 CAR-T cells increased in number during the antileukemia response, and could readily be detected in peripheral blood at multiple time points; confirming persistence for >3 weeks after adoptive transfer (FIG. 31B). Serial bioluminescence imaging confirmed the strong antileukemia activity in all mice with FLT3 CAR-T cell engraftment, whereas mice with CAR-T cell engraftment failure, mice that had been treated with control T cells and untreated mice showed rapid leukemia progression (FIG. 31A, 32A). Further flow cytometric analyses confirmed sustained complete remission of AML cells from bone marrow and spleen (FIG. 33A). Kaplan-Meier analysis showed significantly longer overall survival after treatment with FLT3 CAR-T cells compared with control T cells and no treatment (p<0.05) (FIG. 32B). Of note, in all mice that had responded to FLT3 CAR-T cell therapy, we also observed recurrence of extramedullary late disease, consistent with previous reports of CAR-T cell therapy in NSG mouse models^{37, 43} (FIG. 31A). Expression of FLT3 on AML cells from extramedullary late disease manifestations was detectable at similar levels as on native MOLM-13 cells, i.e. antigen loss had not occurred. In aggregate, our data show that FLT3 CAR-T cells confer potent antileukemia activity against FLT3 wild-type and FLT3-ITD⁺ AML cell lines and primary AML cells in vitro and in vivo.

Midostaurin Induces Increased FLT3 Surface Protein Expression in FLT3-ITD⁺ AML Cells

An observation from clinical studies in patients with FLT3-ITD⁺ AML is upregulation of FLT3 as a compensatory mechanism of AML blasts to counteract the effect of FLT3 inhibitors—a mechanism that we hypothesized could be exploited to enhance the antileukemia efficacy of FLT3 CAR-T cells^{24, 25}. We cultured native MOLM-13 AML cells (MOLM-13^{Native}) (FLT3-ITD^{+/-}) in the presence of the FLT3 inhibitor midostaurin)(MOLM-13^{mido}) using a 10-nM dose. We analyzed FLT3 expression on MOLM-13^{mido} by flow cytometry after 2-3 weeks of exposure to the drug and indeed observed significantly higher levels of FLT3 surface protein as assessed by MFI compared to MOLM-13^{native} cells (n=2 experiments, p<0.05) (FIG. 10A). Further, we slowly increased midostaurine concentration from 10 nM to 50 nM in next 8-10 weeks and observed further increase in FLT3 expression (FIG. 10B). Interestingly, withdrawal of midostaurin led to a decrease in FLT3 expression on MOLM-13 cells to baseline or slightly below baseline levels within 2 days, but increased again upon re-exposure to the drug (FIG. 10C). After primary exposure to midostaurin, we observed a moderate cytotoxic effect and slower expansion of MOLM-13^{mido} cells compared to MOLM-13^{native} cells for approx. 2 weeks. However, despite continuous supplementation to the culture medium, the cytotoxic effect of midostaurin subsequently ceased and the expansion of MOLM-13^{mido} cells accelerated, suggesting they had acquired resistance.

An increase in FLT3 expression upon exposure to midostaurin was also observed with MV4;11 AML cells (FLT3-ITD^{+/+}), but did not occur in several cell lines expressing wild-type FLT3, i.e. THP-1 AML cells, K562 erythro-myeloid leukemia, suggesting upregulation of FLT3 expression in response to midostaurin treatment specifically occurred in FLT3-ITD⁺ AML cells (FIG. 10A,B).

Higher FLT3 Expression on AML MOLM-13^{mido} Cells Leads to Enhanced Antileukemia Reactivity of FLT3 CAR-T Cells In Vitro

We observed significantly higher cytolytic activity of CD8⁺ FLT3 CAR-T cells against MOLM-13^{mido} (90.3±1.9) compared to native MOLM-13^{native} cells (79.4±2.9) at 10:1 E:T ratio (p<0.05) (FIG. 34). Further at physiologically relevant E:T ratio, we observed 1.3 fold (84.5±1.8 vs 64.6±4.1 at 5:1 E:T ratio) and 1.6 fold (59.1±5.5 vs 36.1±2.3 at 2.5:1 E:T ratio) increase in cytolytic activity of CD8⁺ FLT3 CAR-T cells (FIG. 34). Next, we analyzed specific cytokine production by FLT3 CAR-T cells against MOLM-13^{mido} compared to native MOLM-13^{native} cells. Indeed, We observed 2.1 fold higher (MOLM-13^{mido} vs MOLM-13^{native}, 3079.0±153.0 vs 1477.0±78.0 pg/mL) IFN-γ production and 6.6 fold higher (MOLM-13^{mido} vs MOLM-13^{native} 1328.0±63.0 vs 202.0±41.0 pg/mL) IL-2 production by FLT3-CAR T cells (FIG. 35A). FLT3 CAR T cells proliferated 1.8 fold (proliferation index) higher against MOLM-13^{mido} (% proliferation, MOLM-13^{mido} vs MOLM-13^{native}, 75.1 vs 41.2) compared to native MOLM-13^{native} cells (FIG. 35B). The percentage of T cells that proliferated at least 4 and at least 5 times after stimulation with MOLM-13^{mido} was 28.3 and 32.9 as compared to 13.4 and 15.1 against MOLM-13^{native} respectively (FIG. 35B), demonstrating a significant gain of function.

Crenolanib Induces Increased FLT3 Surface Protein Expression in FLT3-ITD⁺ AML Cells

An observation from clinical studies in patients with FLT3-ITD⁺ AML is upregulation of FLT3 as a compensatory mechanism of AML blasts to counteract the effect of FLT3 inhibitors—a mechanism that we hypothesized could be exploited to enhance the antileukemia efficacy of FLT3 CAR-T cells^{24, 25}. We cultured native MOLM-13 AML cells (MOLM-13^{native}) (FLT3-ITD^{+/-}) in the presence of the FLT3 inhibitor crenolanib (MOLM-13^{creno}) using a 10-nM dose, which is a clinically achievable serum level^{22, 44}. We analyzed FLT3 expression on MOLM-13^{creno} by flow cytometry after 5 days of exposure to the drug and indeed observed significantly higher levels of FLT3 surface protein as assessed by MFI compared to MOLM-13^{native} cells (n=3 experiments, p<0.05) (FIG. 14A). Interestingly, withdrawal of crenolanib led to a decrease in FLT3 expression on MOLM-13 cells to baseline levels within 2 days, but increased again upon re-exposure to the drug (FIG. 14B). After primary exposure to crenolanib, we observed a moderate cytotoxic effect and slower expansion of MOLM-13^{creno} cells compared to MOLM-13^{native} cells for approx. 7 days. However, despite continuous supplementation to the culture medium, the cytotoxic effect of crenolanib subsequently ceased and the expansion of MOLM-13^{creno} cells accelerated, suggesting they had acquired resistance.

Higher FLT3 Expression on AML MOLM-13^{creno} Cells Leads to Enhanced Antileukemia Reactivity of FLT3 CAR-T Cells In Vitro

We observed significantly higher cytolytic activity of CD8⁺ FLT3 CAR-T cells against MOLM-13^{creno} (81.4±2.0) compared to native MOLM-13^{native} cells (63.4±5.3) at 10:1 E:T ratio (p<0.05) (FIG. 36). Further at physiologically relevant E:T ratio, we observed 1.6 fold (67.0±2.4 vs 41.9±9.0 at 5:1 E:T ratio) and 1.8 fold (56.8±1.8 vs 30.5±4.7 at 2.5:1 E:T ratio) increase in cytolytic activity of CD8⁺ FLT3 CAR-T cells (FIG. 36). Next, we analyzed specific cytokine production by FLT3 CAR-T cells against MOLM-13^{creno} compared to native MOLM-13^{native} cells. Indeed, We observed 1.6 fold higher (MOLM-13^{creno} vs MOLM-13^{native}, 2413.5±79.3 vs 1477.1±110.4 pg/mL) IFN-γ production and 2.0 fold higher (MOLM-13^{creno} vs MOLM-13^{native}, 642.0±177.1 vs 317.6±105.7 pg/mL) IL-2 production by FLT3-CAR T cells (FIG. 37A). FLT3 CAR T cells proliferated 1.3 fold (proliferation index) higher against MOLM-13^{creno} (% proliferation, MOLM-13^{creno} vs MOLM-13^{native}, 73.0 vs 56.5) compared to native MOLM-13^{native} cells (FIG. 37B). The percentage of T cells that proliferated at least 4 and at least 5 times after stimulation with MOLM-13^{creno} was 16.6 and 25.2 as compared to 9.5 and 17.3 against MOLM-13^{native} respectively (FIG. 37B), demonstrating a significant gain of function.

Quizartinib Induces Increased FLT3 Surface Protein Expression in FLT3-ITD⁺ AML Cells

An observation from clinical studies in patients with FLT3-ITD⁺ AML is upregulation of FLT3 as a compensatory mechanism of AML blasts to counteract the effect of FLT3 inhibitors—a mechanism that we hypothesized could be exploited to enhance the antileukemia efficacy of FLT3 CAR-T cells^{24, 25}. We cultured native MOLM-13 AML cells (MOLM-13^{native}) FLT3-ITD^{+/-}) in the presence of the FLT3 inhibitor quizartinib (MOLM-13^{Quiza}) using a 1-nM dose, which is a clinically achievable serum level^{27, 44}. We analyzed FLT3 expression on MOLM-13^{quiza} by flow cytometry after 5 days of exposure to the drug and indeed observed significantly higher levels of FLT3 surface protein as assessed by MFI compared to MOLM-13^{native} cells (n=3 experiments, p<0.05) (FIG. 19A). Interestingly, withdrawal of quizartinib led to a decrease in FLT3 expression on MOLM-13 cells to baseline levels within 2 days, but increased again upon re-exposure to the drug (FIG. 19B). After primary exposure to quizartinib, we observed a moderate cytotoxic effect and slower expansion of MOLM-13^{quiza} cells compared to MOLM-13^{native} cells for approx. 7 days. However, despite continuous supplementation to the culture medium, the cytotoxic effect of quizartinib subsequently ceased and the expansion of MOLM-13^{quiza} cells accelerated, suggesting they had acquired resistance.

An increase in FLT3 expression upon exposure to quizartinib was also observed with MV4;11 AML cells (FLT3-ITD^{+/+}), but did not occur in several cell lines expressing wild-type FLT3, i.e. THP-1 AML cells, JeKo-1 mantle cell lymphoma, and K562 erythro-myeloid leukemia, suggesting upregulation of FLT3 expression in response to quizartinib treatment specifically occurred in FLT3-ITD⁺ AML cells (FIG. 19B).

Higher FLT3 Expression on AML MOLM-13^{quiza} Cells Leads to Enhanced Antileukemia Reactivity of FLT3 CAR-T Cells In Vitro

We observed significantly higher cytolytic activity of CD8⁺ FLT3 CAR-T cells against MOLM-13^{quiza} (72.4±3.9) compared to native MOLM-13^{native} cells (54.4±1.7) at 10:1 E:T ratio (p<0.05) (FIG. 38). Further at physiologically relevant E:T ratio, we observed we observed 1.6 fold (42.6±3.9 vs 27.0±5.6 at 5:1 E:T ratio) and 3.8 fold (24.9±4.5 vs 6.6±7.0 at 2.5:1 E:T ratio) increase in cytolytic activity of CD8⁺ FLT3 CAR-T cells (FIG. 38). Next, we analyzed specific cytokine production by FLT3 CAR-T cells against MOLM-13^{quiza} compared to native MOLM-13^{native} cells. Indeed, We observed 1.2 fold higher (MOLM-13^{quiza} vs MOLM-13^{native}, 1839.0±11.0 vs 1477.0±78.0 pg/mL) IFN-γ production and 1.9 fold higher (MOLM-13^{quiza} vs MOLM-13^{native}, 376.0±10.0 vs 202.0±41.0 pg/mL) IL-2 production by FLT3-CAR T cells (FIG. 39A). FLT3 CAR T cells proliferated 1.2 fold (proliferation index) higher against MOLM-13^{quiza} (% proliferation, MOLM-13^{quiza} vs MOLM-13^{native}, 65.0 vs 56.5) compared to native MOLM-13^{native} cells (FIG. 39B). The percentage of T cells that proliferated at least 4 and at least 5 times after stimulation with MOLM-13^{quiza} was 14.2 and 22.5 as compared to 9.5 and 17.3 against MOLM-13^{native} respectively (FIG. 39B), demonstrating a significant gain of function.

### Example 3

Materials and Methods:

Human Subjects

Peripheral blood was obtained from healthy donors after written informed consent to participate in research protocols approved by the Institutional Review Board of the University of Würzburg.

Tumor Cell Lines

The human leukemia cell lines MOLM-13 (ACC 554) was purchased from DSMZ (Deutsche Sammlung von Mikroorganismen and Zellkulturen, Braunschweig, Germany) and cultured in RPMI-1640 supplemented with 10% fetal calf serum (FCS), 2 mM glutamine and 100 U/mL penicillin/streptomycin. MOLM-13 cells were transduced with a lentiviral vector encoding a firefly luciferase (ffluc)_green fluorescent protein (GFP) transgene to enable detection by flow cytometry (GFP) and bioluminescence imaging (ffLuc) in mice, and bioluminescence-based cytotoxicity assays.

Flow Cytometric Analysis of FLT3 Expression

Cell surface expression of FLT3 was analyzed using a conjugated mouse-anti-human-FLT3 mAb (clone 4G8, BD Biosciences, Germany) and mouse IgG1 isotype control (BD). In brief, 1×10⁶ cells were washed, resuspended in 100 μL PBS/0.5% fetal calf serum and stained with 5 μL of anti-FLT3 mAb or isotype for 30 minutes at 4° C.

CAR Construction

A codon optimized targeting domain comprising the V_{H} and V_{L} segments of the FLT3-specific BV10 mAb¹² was synthesized (GeneArt, ThermoFisher, Regensburg, Germany) and fused to a CAR backbone comprising a short IgG4-Fc Hinge spacer, a CD28 transmembrane and costimulatory moiety and CD3z, in-frame with a T2A element and EGFRt transduction marker (FIG. 1)³²⁻³⁴. The entire transgene was encoded in a lentiviral vector epHIV7 and expressed under control of an EF1/HTLV hybrid promotor^{34, 35}.

Preparation of CAR-Modified T Cells

Lentiviral gene-transfer was performed into CD3/28-bead (ThermoFisher) activated CD4⁺ and CD8⁺ T cells on day 1 after bead stimulation at MOI of 5. T cells were cultured in RPMI-1640 supplemented with 10% human serum, glutamine, 2 mM glutamine, 100 U/mL penicillin/streptomycin and 50 U/mL recombinant human interleukin (IL)-2 (Proleukine, Novartis, Basel, Switzerland)³². CAR-transduced T cells were enriched using biotinylated anti-EGFR mAb (ImClone Systems Inc.) and anti-biotin beads (Miltenyi), prior to expansion using a rapid expansion protocol³⁸.

Flow Cytometric Analyses of T Cells

CAR-modified and untransduced T cells were stained with 1 or more of the following conjugated mAbs: CD3, CD4, CD8 and 7-AAD for live/dead cell discrimination (Miltenyi/BD/Biolegend). CAR-transduced (i.e. EGFRt⁺) T-cells were detected by staining with anti-EGFR antibody that had been biotinylated in-house (EZ-Link™Sulfo-NHS-SS-Biotin, Thermofisher Scientific, IL, according to the manufacturer's instructions) and streptavidin-PE. Flow analyses were done on a FACSCanto (BD) and data analyzed using Flowio software v9.0.2 (Treestar, Ashland, Oreg.).

In Vivo Experiments

All experiments were approved by the Institutional Animal Care and Use Committees of the participating institutions. NOD.Cg-Prkdc^{scid} Il2rg^{tm1WjI}/SzJ (NSG) mice (female, 6-8 week old) were purchased from Charles River or bred in-house. Mice were inoculated with 1×10⁶ ffluc_GFP⁺MOLM-13 AML cells by tail vein injection on day 0, and received a single dose of 5×10⁶ T cells (in 200 μL of PBS/0.5% FCS) by tail vein injection on day 7. Quizartinib [1 mg/kg; 200 μL of 30% glycerol formal] or midostaurin [1 mg/kg; 200 μL of 30% glycerol formal] was administered intraperitoneally (i.p.) Monday-Friday for 3 consecutive weeks (total of 15 doses). AML progression/regression was assessed by serial bioluminescence imaging following i.p. administration of D-luciferin substrate (0.3 mg/g body weight) (Biosynth, Staad, Switzerland) using an IVIS Lumina imaging system (Perkin Elmer, Waltham, Mass.). Data was analyzed using Living Image software (Perkin Elmer).

Pharmaceutical Drugs and Reagents

Quizartinib and midostaurin (SelleckChemicals, Houston, Tex.) were reconstituted in dimethylsulfoxide (DMSO) prior to dilution in 30% glycerol formal (Sigma Aldrich, Munich, Germany) and use in the in vivo experiments.

Statistical Analyses

Statistical analyses were performed using Prism software v6.07 (GraphPad). Unpaired Student's t-tests were used for analysis of data. Log-rank (Mantel-Cox) testing was performed to analyze differences in survival observed in in vivo experiments. Differences with a p value <0.05 were considered statistically significant.

Results:

Midostaurin Acts Synergistically with FLT3 CAR-T Cells In Vivo

We examined the anti-leukemia effect of FLT3 CAR-T cells in combination with midostaurin in vivo. We inoculated mice with MOLM-13^{Native} AML cells and treated with them either FLT3 CAR-T cells alone, midostaurin alone, the combination treatment with FLT3 CAR-T cells and midostaurin, or left mice untreated. The combination treatment was administered with 2 different schedules: One group of mice received midostaurin already from day 3 after leukemia inoculation (FLT3 CAR+early midostaurin, i.e. midostaurin administration commenced even prior to FLT3 CAR-T cell transfer) and the other group of mice received midostaurin from day 7 after leukemia inoculation (FLT3 CAR+midostaurin, i.e. midostaurin administration commenced at the day of FLT3 CAR-T cell transfer). In both groups, a total of 15 doses of midostaurin were administered.

We observed potent anti-leukemia efficacy in mice receiving the combination treatment with FLT3 CAR-T cells and midostaurin (FIG. 40a, b). In comparison to mice that were treated with FLT3 CAR-T cells only, we observed superior engraftment and in vivo expansion of FLT3 CAR-T cells in mice that received the combination therapy (FIG. 41a). The mean frequency of FLT3 CAR-T cells in mice that received FLT3 CAR-T cells+midostaurin was more than twice as high compared to mice that had received FLT3 CAR-T cells alone (>100% increase) (p<0.05).

Further, we observed faster and deeper remissions in mice treated with combination therapy as assessed by bioluminescence imaging (FIG. 40b). In the group of mice, that had received midostaurin only, we did not observe a reduction in leukemia burden in any of the mice (response rate: 0/4=0%). In the group of mice, that had received FLT3 CAR-T cells only, we observed leukemia reduction in all of the mice (4/4=100%) however, in none of the mice was the reduction in BL signal (as a marker for leukemia regression) greater than 50-fold (0/4 mice=0%). In the group of mice that had received FLT3 CAR-T cells+early midostaurin, we observed leukemia reduction in all of the mice (4/4=100%), and in 3 out of 4=75% of mice was leukemia regression greater than 50-fold. In the group of mice that had received FLT3 CAR-T cells+midostaurin, we observed leukemia reduction in all of the mice (4/4=100%) and in 4 out of 4=100% of mice was leukemia regression greater than 50-fold. The strongest anti-leukemia response was observed in the group of mice that had received FLT3 CAR-T cells+midostaurin.

We analyzed FLT3 expression on MOLM-13 cells that we recovered from bone marrow and found that FLT3 was strongly upregulated in mice that had received midostaurin compared to mice that had not received midostaurin (FIG. 41b). In particular, the MFI in the mouse with the lowest level of FLT3 expression was 30% higher in the group of mice that had received midostaurin compared to the mouse that had not received midostaurin.

In summary, the data show that midostaurin exerts synergistic anti-leukemia activity in combination with FLT3 CAR-T cells.

Quizartinib Acts Synergistically with FLT3 CAR-T Cells In Vivo

We examined the anti-leukemia effect of FLT3 CAR-T cells in combination with quizartinib in the NSG/MOLM-13 xenograft model in vivo. Mice received a single dose of FLT3 CAR-T cells alone, quizartinib alone (1 mg/kg, i.p), the combination treatment with FLT3 CAR-T cells and quizartinib, or were left untreated.

We observed potent anti-leukemia efficacy in mice receiving the combination treatment with FLT3 CAR-T cells and quizartinib (FIG. 42a, b). In comparison to mice treated with only CAR-T cells, we observed superior engraftment and significantly higher in vivo expansion of FLT3 CAR-T cells in mice treated with quizartinib and FLT3 CAR-T cells combination therapy (FIG. 43a). The mean frequency of FLT3 CAR-T cells in mice that received FLT3 CAR-T cells+ quizartinib was nearly twice as high compared to mice that had received FLT3 CAR-T cells alone (>85% increase) (p<0.0001).

Furthermore, we observed faster and deeper remissions in mice treated with combination therapy as evaluated by bioluminescence imaging (FIG. 42b). In the group of mice, that had received quizartinib only, we did not observe a reduction in leukemia burden in any of the mice (response rate: 0/4=0%). In the group of mice, that had received FLT3 CAR-T cells only, we observed leukemia reduction in all of the mice (6/6=100%) however, only in 2 of the mice was the reduction in BL signal (as marker for leukemia regression) greater than 20-fold (2/6 mice=33%). In the group of mice that had received FLT3 CAR-T cells+ quizartinib, we observed leukemia reduction in all of the mice (6/6=100%) and in 5 out of 6=83.3% of mice was leukemia regression greater than 20-fold. The strongest anti-leukemia response was observed in the group of mice that had received FLT3 CAR-T cells+ quizartinib.

We analyzed FLT3 expression on MOLM-13 cells recovered from bone marrow and observed increased FLT3 expression after quizartinib treatment (FIG. 43b). In particular, the MFI in the mouse with the lowest level of FLT3 expression was approx. 65% higher in the group of mice that had received quizartinib compared to the mouse that had not received quizartinib.

In summary, the data show that quizartinib acts synergistically in mediating regression of leukemia in combination with FLT3 CAR-T cells in vivo.

### Example 4

Human Subjects

Peripheral blood was obtained from healthy donors after written informed consent to participate in research protocols approved by the Institutional Review Board of the University of Würzburg.

Tumor Cell Lines

The human leukemia cell lines NALM-16 (ACC 680), KOPN-8 (ACC 552), SEM (ACC 546) were purchased from DSMZ (Deutsche Sammlung von Mikroorganismen and Zellkulturen, Braunschweig, Germany) and cultured in RPMI-1640 supplemented with 10% fetal calf serum (FCS), 2 mM glutamine and 100 U/mL penicillin/streptomycin. All cell lines were transduced with a lentiviral vector encoding a firefly luciferase (ffluc)_green fluorescent protein (GFP) transgene to enable detection by flow cytometry (GFP) and bioluminescence imaging (ffLuc) in mice, and bioluminescence-based cytotoxicity assays.

Flow Cytometric Analysis of FLT3 Expression

Cell surface expression of FLT3 (CD135) was analyzed using a conjugated mouse-anti-human-FLT3 mAb (clone 4G8, BD Biosciences, Germany) and mouse IgG1 isotype control (BD). In brief, 1×10⁶ cells were washed, resuspended in 100 μL PBS/0.5% fetal calf serum and stained with 5 μL of anti-FLT3 mAb or isotype for 30 minutes at 4° C.

CAR Construction

A codon optimized targeting domain comprising the V_{H} and V_{L} segments of the FLT3-specific BV10 mAb¹² was synthesized (GeneArt, ThermoFisher, Regensburg, Germany) and fused to a CAR backbone comprising a short IgG4-Fc Hinge spacer, a CD28 transmembrane and costimulatory moiety and CD3z, in-frame with a T2A element and EGFRt transduction marker (FIG. 1)³²⁻³⁴. The entire transgene was encoded in a lentiviral vector epHIV7 and expressed under control of an EF1/HTLV hybrid promotor^{34, 35}. Similarly, targeting domains specific for CD19 (clone FMC63) was used to generate CD19^{32, 33, 36, 37}.

Preparation of CAR-Modified T Cells

Lentiviral gene-transfer was performed into CD3/28-bead (ThermoFisher) activated CD4⁺ and CD8⁺ T cells on day 1 after bead stimulation at a moiety of infection (MOI) of 5. T cells were cultured in RPMI-1640 supplemented with 10% human serum, glutamine, 2 mM glutamine, 100 U/mL penicillin/streptomycin and 50 U/mL recombinant human interleukin (IL)-2 (Proleukine, Novartis, Basel, Switzerland)³². CAR-transduced T cells were enriched using biotinylated anti-EGFR mAb (ImClone Systems Inc.) and anti-biotin beads (Miltenyi), prior to expansion using a rapid expansion protocol³⁸ or—for CD19 CAR-T cells—using antigen-specific stimulation with irradiated (80 Gy) CD19⁺ feeder cells³⁸.

Flow Cytometric Analyses of T Cells

CAR-modified and untransduced T cells were stained with 1 or more of the following conjugated mAbs: CD3, CD4, CD8 and 7-AAD for live/dead cell discrimination (Miltenyi/BD/Biolegend). CAR-transduced (i.e. EGFRt⁺) T-cells were detected by staining with anti-EGFR antibody that had been biotinylated in-house (EZ-Link™Sulfo-NHS-SS-Biotin, Thermofisher Scientific, IL, according to the manufacturer's instructions) and streptavidin-PE. Flow analyses were done on a FACSCanto (BD) and data analyzed using FlowJo software v9.0.2 (Treestar, Ashland, Oreg.).

Analysis of CAR-T Cell Function In Vitro

Functional analyses were performed as previously described^{32, 33, 39-41}. In brief, target cells expressing firefly luciferase (ffLuc) were incubated in triplicate at 5×10³ cells/well with effector T-cells at various effector to target (E:T) ratios. After 4-hour incubation, luciferin substrate was added to the co-culture and the decrease in luminescence signal in wells that contained target cells and T-cells was measured using a luminometer (Tecan, Männedorf, Switzerland) and compared to target cells alone. Specific lysis was calculated using the standard formula⁴². For analysis of cytokine secretion, 50×10³ T-cells were plated in triplicate wells with target cells at a ratio of 2:1 and IFN-γ and IL-2 production measured by ELISA (Biolegend) in supernatant removed after 24-hour incubation. For analysis of proliferation, 50×10³ T-cells were labeled with 0.2 μM carboxyfluorescein succinimidyl ester (CFSE, ThermoFisher), washed and plated in triplicate wells with target cells at a ratio of 2:1 in medium without exogenous cytokines. After 72-hour incubation, cells were labeled with anti-CD8/CD4 mAb and 7-AAD to exclude dead cells from analysis. Samples were analyzed by flow cytometry and division of live T-cells assessed by CFSE dilution.

FLT3 Inhibitor Treatment of Leukemia Cells

Acute lymphoblastic leukemia (NALM-16) and mixed lineage leukemia (KOPN-8 and SEM) cells were maintained in RPMI-1640 medium, supplemented with 10% fetal calf serum, 2 mM glutamine, 100 U/mL penicillin/streptomycin, and 10 nM crenolanib or 1 nM quizartinib or 50 nM midostaurin. 1×10⁶/mL NALM-16, KOPN-8 and SEM cell suspension plated per well in 24-well plates (Costar, Corning, NJ). After a week of culture with 10 nM crenolanib or 1 nM quizartinib or 50 nM midostaurin, cells were stained with anti-FLT3 4G8 mAb and flow cytometry analysis was carried out.

Pharmaceutical Drugs and Reagents

Crenolanib, quizartinib and midostaurin (SelleckChemicals, Houston, Tex.) were reconstituted in dimethylsulfoxide (DMSO) prior to dilution in medium or 30% glycerol formal (Sigma Aldrich, Munich, Germany) and use in the in vitro or in vivo experiments, respectively.

Statistical Analyses

Statistical analyses were performed using Prism software v6.07 (GraphPad). Unpaired Student's t-tests were used for analysis of data obtained in in vitro experiments. Differences with a p value <0.05 were considered statistically significant.

Results:

FLT3 CAR-T Cells Mediate Potent Anti-Leukemia Activity Against ALL and MLL In Vitro

FLT3 expression has been reported in patients with acute lymphoblastic leukemia (ALL) and mixed lineage leukemia (MLL)^{1,4,47}. Therefore, we sought to determine whether FLT3 CAR-T cells were able to recognize and eliminate ALL and MLL.

To evaluate the reactivity of FLT3 CAR-T cells against ALL and MLL cell lines, we included NALM-16 (wt FLT3+, CD19+ pediatric ALL), KOPN-8 (wt FLT3+, CD19+ infant MLL with KMT2A-MLLT1 fusion gene) and SEM (wt FLT3+, CD19+ pediatric MLL with KMT2A-AFF1 fusion gene) into our analyses. First, we confirmed FLT3 expression by all three cell lines using flow cytometry (FIG. 44a). Then we carried out functional analyses and observed specific high-level cytolytic activity of CD8+FLT3 CAR-T cells at multiple effector to target cell ratios (range: 10:1-2.5:1) against all three cell lines (FIG. 44b). Further, CD4+FLT3 CAR-T and CD19 CAR-T cells produced significant amount of IL-2 and underwent antigen specific proliferation after stimulation with all three target cell lines, whereas control T cells did not show any proliferation (FIG. 45a, b).

Next, we analyzed whether FLT3 expression on ALL and MLL cells can be enhanced by treatment with FLT3 inhibitors. Therefore, we exposed wild type FLT3 expressing ALL and MLL cells to FLT3 inhibitors for 7 days. However, we did not observe an increase in FLT3 expression on these cells within the assay period (FIG. 46a).

In summary, the results show that FLT3 CAR-T cells exert specific anti-leukemia activity against FLT3 positive ALL and MLL and can be exploited to treat ALL and MLL patients.

The fact that an increase in FLT3 expression on the FLT3 wild-type cells after treatment with the tested FLT3 inhibitors was not observed within the assay period (FIG. 46a) does not rule out that increase in FLT3 expression can occur in wild-type FLT3-expressing cancers after long-term treatments with FLT3 inhibitors (as seen with CEP701)²⁴. In fact, given that wild-type FLT3 is known to be highly expressed in several cancers, it is expected that a treatment of wild-type FLT3-expressing cancers with FLT3 inhibitors will lead to a long-term adaptation of the cancer cells (e.g. by genetic selection) that counteracts the FLT3 inhibition by mechanisms which include further increases in wild-type FLT3 expression. According to the invention, a treatment with an FLT3-targeting agent (e.g. CAR-modified cell such as a CAR-T cell) will be particularly effective in such cancers which exhibit (further) increases in wild-type FLT3 expression. It is therefore expected that the combination treatments with a kinase inhibitor according to the invention (e.g. an FLT3 inhibitor according to the invention) and with an FLT3-targeting agent according to the invention (e.g. CAR-modified cell according to the invention such as a CAR-T cell) will be effective and will exhibit synergistic effects of the kinase inhibitor and the FLT3-targeting agent in cancers expressing wild-type FLT3. Additionally, it is also expected that said combination treatments according to the invention will be effective to prevent, or effectively treat, a situation where a wild-type FLT3-expressing cancer acquires an FLT3 mutation during the course of a treatment with an FLT3 inhibitor.

Accordingly, the invention can be applied advantageously to cancers including any cancers expressing wild-type FLT3 and/or mutated FLT3.

### Example 5

Human Subjects

Peripheral blood was obtained from healthy donors after written informed consent to participate in research protocols approved by the Institutional Review Board of the University of Würzburg.

FLT3 Inhibitor Treatment of MV4;11 AML Cells

MV4;11 cells were maintained in RPMI-1640 medium, supplemented with 10% fetal calf serum, 2 mM glutamine, 100 U/mL penicillin/streptomycin, and 10 nM crenolanib or 1 nM quizartinib or 50 nM midostaurin. MV4;11 cells were plated at a concentration of 1×10⁶/mL in 1 mL per well in 24-well plates (Costar, Corning, NJ).

Antibody-Dependent Cellular Cytotoxicity (ADCC) Assay

MV4;11 AML cells transduced with firefly luciferase (ffluc) were utilized for ADCC assays. MV4;11 AML cells were pre-treated with FLT3 inhibitors for 7 days or were left untreated. Target cells were co-incubated with healthy donor derived PBMCs at an effector-to-target ratio of 50:1 in triplicate wells of 96-well flat-bottom plates in the presence of solvent control, IgG1 isotype control (5000 ng/mL) or anti-FLT3 BV10 mAb (5000 ng/mL) (Biolegend, London, UK). ADCC was determined in a bioluminescence-based assay after 24 hours⁴⁰. Luciferin substrate was added to the co-culture and the decrease in luminescence signal was measured using a luminometer (Tecan, Männedorf, Switzerland). The percentage of viable cells was calculated using the following formula: % viability=bioluminescence signal in the presence of effector cells and BV10 mAb (with or without FLT3 inhibitor pre-treatment)×100/bioluminescence signal in the control condition.

Pharmaceutical Drugs and Reagents

Crenolanib, quizartinib and midostaurin (SelleckChemicals, Houston, Tex.) were reconstituted in dimethylsulfoxide (DMSO) prior to dilution in medium and use in the in vitro experiments.

Statistical Analyses

Statistical analyses were performed using Prism software v6.07 (GraphPad). Unpaired Student's t-tests were used for analysis of data obtained in in vitro experiments. Differences with a p value <0.05 were considered statistically significant.

Results:

FLT3 Inhibitors Act Synergistically with Anti-FLT3 mAb

We sought to determine whether the increase in FLT3 antigen density on AML cells after FLT3 inhibitor treatment enabled superior anti-leukemia activity of anti-FLT3 mAbs. Therefore, we treated MV4;11 AML cells with FLT3 inhibitors (10 nM crenolanib, 1 nM quizartinib or 50 nM midostaurin) for 7 days. We then carried out an ADCC assay using the anti-FLT3 mAb BV10. A matched isotype antibody served as control. MV4;11 target cells (untreated or FLT3 inhibitor treated) and healthy donor derived PBMC were co-cultured in the presence or absence of BV10 mAb, and the decrease in viable MV4;11 cells determined after 24 hours. We observed a significant increase in ADCC against FLT3 inhibitor treated MV4;11 cells compared to untreated MV4;11 cell (FIG. 47). On average, 39% of crenolanib-treated MV4;11 cells, 46% of quizartinib-treated MV4;11 cells and 26% of midostaurin-treated MV4;11 cells were eliminated by BV10 mAb within the 24-hour ADCC assay, whereas only 13% MV4;11 cells were eliminated without FLT3 inhibitor pre-treatment (p<0.05) (FIG. 47). These data show that the percentage of MV4;11 cells that was eliminated within the 24-hour assay period was 3-fold higher after crenolanib pre-treatment (39% with crenolanib pretreatment vs. 13% without pretreatment); 3.5-fold higher after quizartinib pre-treatment (46% with quizartinib pretreatment vs. 13% without pretreatment); and 2-fold higher after midostaurin pre-treatment (26% with midostaurin pretreatment vs. 13% without pretreatment). In summary, the data show that FLT3 inhibitors exert synergistic anti-leukemic activity in combination with anti-FLT3 mAb.

### INDUSTRIAL APPLICABILITY

The inhibitors and targeting agents, the combination of these, the compositions and formulations, as well as the kits according to the present invention may be industrially manufactured and sold as products for the claimed methods and uses (e.g. for treating a cancer as defined herein), in accordance with known standards for the manufacture of pharmaceutical products. Accordingly, the present invention is industrially applicable.

### REFERENCES

1. Gilliland D G, Griffin J D. The roles of FLT3 in hematopoiesis and leukemia. Blood 2002; 100(5): 1532-1542.
2. Kikushige Y, Yoshimoto G, Miyamoto T, Iino T, Mori Y, Iwasaki H, et al. Human Flt3 is expressed at the hematopoietic stem cell and the granulocyte/macrophage progenitor stages to maintain cell survival. The Journal of Immunology 2008; 180(11): 7358-7367.
3. Böiers C, Buza-Vidas N, Jensen C T, Pronk C J, Kharazi S, Wittmann L, et al. Expression and role of FLT3 in regulation of the earliest stage of normal granulocyte-monocyte progenitor development. Blood 2010; 115(24): 5061-5068.
4. Rosnet O, Bühring H, Marchetto S, Rappold I, Lavagna C, Sainty D, et al. Human FLT3/FLK2 receptor tyrosine kinase is expressed at the surface of normal and malignant hematopoietic cells. Leukemia 1996; 10(2): 238-248.
5. Karsunky H, Merad M, Cozzio A, Weissman I L, Manz M G. Flt3 ligand regulates dendritic cell development from FIt3+ lymphoid and myeloid-committed progenitors to Flt3+ dendritic cells in vivo. Journal of Experimental Medicine 2003; 198(2): 305-313.
6. Park I-K, Trotta R, Yu I, Caligiuri M A. Axl/Gas6 pathway participates in human natural killer cell development by positively regulating FLT3 activation. European journal of immunology 2013; 43(10).
7. Waskow C, Liu K, Darrasse-Jèze G, Guermonprez P, Ginhoux F, Merad M, et al. The receptor tyrosine kinase Flt3 is required for dendritic cell development in peripheral lymphoid tissues. Nature immunology 2008; 9(6): 676-683.
8. Carow C E, Levenstein M, Kaufmann S H, Chen J, Amin S, Rockwell P, et al. Expression of the hematopoietic growth factor receptor FLT3 (STK-1/Flk2) in human leukemias. Blood 1996; 87(3): 1089-1096.
9. Ozeki K, Kiyoi H, Hirose Y, Iwai M, Ninomiya M, Kodera Y, et al. Biologic and clinical significance of the FLT3 transcript level in acute myeloid leukemia. Blood 2004; 103(5): 1901-1908.
10. Vora H H, Shukla S N, Brahambhatt B V, Mehta S H, Patel N A, Parikh S K, et al. Clinical relevance of FLT3 receptor protein expression in Indian patients with acute leukemia. Asia-Pacific Journal of Clinical Oncology 2010; 6(4): 306-319.
11. Kindler T, Lipka D B, Fischer T. FLT3 as a therapeutic target in AML: still challenging after all these years. Blood 2010; 116(24): 5089-5102.
12. Hofmann M, Große-Hovest L, Nübling T, Pyż E, Bamberg M, Aulwurm S, et al. Generation, selection and preclinical characterization of an Fc-optimized FLT3 antibody for the treatment of myeloid leukemia. Leukemia 2012; 26(6): 1228-1237.
13. Stone J D, Aggen D H, Schietinger A, Schreiber H, Kranz D M. A sensitivity scale for targeting T cells with chimeric antigen receptors (CARS) and bispecific T-cell Engagers (BiTEs). Oncoimmunology 2012; 1(6): 863-873.
14. Kuchenbauer F, Kern W, Schoch C, Kohlmann A, Hiddemann W, Haferlach T, et al. Detailed analysis of FLT3 expression levels in acute myeloid leukemia. Haematologica 2005; 90(12): 1617-1625.
15. Thiede C, Steudel C, Mohr B, Schaich M, Schäkel U, Platzbecker U, et al. Analysis of FLT3-activating mutations in 979 patients with acute myelogenous leukemia: association with FAB subtypes and identification of subgroups with poor prognosis. Blood 2002; 99(12): 4326-4335.
16. Fröhling S, Scholl C, Levine R L, Loriaux M, Boggon T J, Bernard O A, et al. Identification of driver and passenger mutations of FLT3 by high-throughput DNA sequence analysis and functional assessment of candidate alleles. Cancer cell 2007; 12(6): 501-513.
17. Levis M, Murphy K M, Pham R, Kim K-T, Stine A, Li L, et al. Internal tandem duplications of the FLT3 gene are present in leukemia stem cells. Blood 2005; 106(2): 673-680.
18. Shih L, Huang C, Wang P, Wu J, Lin T, Dunn P, et al. Acquisition of FLT3 or N-ras mutations is frequently associated with progression of myelodysplastic syndrome to acute myeloid leukemia. Leukemia 2004; 18(3): 466-475.
19. Brunet S, Labopin M, Esteve J, Cornelissen J, Socié G, Iori A P, et al. Impact of FLT3 internal tandem duplication on the outcome of related and unrelated hematopoietic transplantation for adult acute myeloid leukemia in first remission: a retrospective analysis. Journal of Clinical Oncology 2012; 30(7): 735-741.
20. Schmid C, Labopin M, Socié G, Daguindau E, Volin L, Huynh A, et al. Outcome of patients with distinct molecular genotypes and cytogenetically normal AML after allogeneic transplantation. Blood 2015; 126(17): 2062-2069.
21. Grunwald M R, Levis M J. FLT3 inhibitors for acute myeloid leukemia: a review of their efficacy and mechanisms of resistance. International journal of hematology 2013; 97(6): 683-694.
22. Alvarado Y, Kantarjian H M, Luthra R, Ravandi F, Borthakur G, Garcia-Manero G, et al. Treatment with FLT3 inhibitor in patients with FLT3-mutated acute myeloid leukemia is associated with development of secondary FLT3-tyrosine kinase domain mutations. Cancer 2014; 120(14): 2142-2149.
23. Heidel F, Solem F K, Breitenbuecher F, Lipka D B, Kasper S, Thiede M, et al. Clinical resistance to the kinase inhibitor PKC412 in acute myeloid leukemia by mutation of Asn-676 in the FLT3 tyrosine kinase domain. Blood 2006; 107(1): 293-300.
24. Knapper S, Burnett A K, Littlewood T, Kell W J, Agrawal S, Chopra R, et al. A phase 2 trial of the FLT3 inhibitor lestaurtinib (CEP701) as first-line treatment for older patients with acute myeloid leukemia not considered fit for intensive chemotherapy. Blood 2006; 108(10): 3262-3270.
25. Weisberg E, Ray A, Nelson E, Adamia S, Barrett R, Sattler M, et al. Reversible resistance induced by FLT3 inhibition: a novel resistance mechanism in mutant FLT3-expressing cells. PloS one 2011; 6(9): e25351.
26. Smith C C, Lasater E A, Lin K C, Wang Q, McCreery M Q, Stewart W K, et al. Crenolanib is a selective type I pan-FLT3 inhibitor. Proceedings of the National Academy of Sciences 2014; 111(14): 5319-5324.
27. Zimmerman E I, Turner D C, Buaboonnam J, Hu S, Orwick S, Roberts M S, et al. Crenolanib is active against models of drug-resistant FLT3-ITD-positive acute myeloid leukemia. Blood 2013; 122(22): 3607-3615.
28. Heinrich M C, Griffith D, McKinley A, Patterson J, Presnell A, Ramachandran A, et al. Crenolanib inhibits the drug-resistant PDGFRA D842V mutation associated with imatinib-resistant gastrointestinal stromal tumors. Clinical Cancer Research 2012; 18(16): 4375-4384.
29. Wetmore C, Broniscer A, Turner D, Wright K D, Pai-Panandiker A, Kun L E, et al. First-in-pediatrics phase I study of crenolanib besylate (CP-868,596-26) administered during and after radiation therapy (RT) in newly diagnosed diffuse intrinsic pontine glioma (DIPG) and recurrent high-grade glioma (HGG). Journal of Clinical Oncology 2014; 32(15_suppl): 10064-10064.
30. Randhawa J K, Kantarjian H M, Borthakur G, Thompson P A, Konopleva M, Daver N, et al. Results of a phase II study of crenolanib in relapsed/refractory acute myeloid leukemia patients (Pts) with activating FLT3 mutations. Blood 2014; 124(21): 389-389.
31. Cortes J. Results from a Phase II Study of Crenolanib in Patients With FLT3-Positive Acute Myeloid Leukemia. ASCO Annual Meeting 2016.
32. Hudecek M, Sommermeyer D, Kosasih P L, Silva-Benedict A, Liu L, Rader C, et al. The nonsignaling extracellular spacer domain of chimeric antigen receptors is decisive for in vivo antitumor activity. Cancer immunology research 2015; 3(2): 125-135.
33. Hudecek M, Lupo-Stanghellini M-T, Kosasih P L, Sommermeyer D, Jensen M C, Rader C, et al. Receptor affinity and extracellular domain modifications affect tumor recognition by ROR1-specific chimeric antigen receptor T cells. Clinical Cancer Research 2013; 19(12): 3153-3164.
34. Wang X, Chang W-C, Wong C W, Colcher D, Sherman M, Ostberg J R, et al. A transgene-encoded cell surface polypeptide for selection, in vivo tracking, and ablation of engineered cells. Blood 2011; 118(5): 1255-1263.
35. Yam P Y, Li S, Wu J, Hu J, Zaia J A, Yee J-K. Design of HIV vectors for efficient gene delivery into human hematopoietic cells. Molecular Therapy 2002; 5(4): 479-484.
36. Monjezi R, Miskey C, Gogishvili T, Schleef M, Schmeer M, Einsele H, et al. Enhanced CAR T-cell engineering using non-viral Sleeping Beauty transposition from minicircle vectors. Leukemia 2017; 31(1): 186-194.
37. Gill S, Tasian S K, Ruella M, Shestova O, Li Y, Porter D L, et al. Preclinical targeting of human acute myeloid leukemia and myeloablation using chimeric antigen receptor-modified T cells. Blood 2014; 123(15): 2343-2354.
38. Riddell S R, Greenberg P D. The use of anti-CD3 and anti-CD28 monoclonal antibodies to clone and expand human antigen-specific T cells. Journal of immunological methods 1990; 128(2): 189-201.
39. Hudecek M, Schmitt T M, Baskar S, Lupo-Stanghellini M T, Nishida T, Yamamoto T N, et al. The B-cell tumor-associated antigen ROR1 can be targeted with T cells modified to express a ROR1-specific chimeric antigen receptor. Blood 2010; 116(22): 4532-4541.
40. Brown C E, Wright C L, Naranjo A, Vishwanath R P, Chang W-C, Olivares S, et al. Biophotonic cytotoxicity assay for high-throughput screening of cytolytic killing. Journal of immunological methods 2005; 297(1): 39-52.
41. Monjezi R, Miskey C, Gogishvili T, Schleef M, Schmeer M, Einsele H, et al. Enhanced CAR T-cell engineering using non-viral Sleeping Beauty transposition from minicircle vectors. Leukemia 2016.
42. Brown C E, Wright C L, Naranjo A, Vishwanath R P, Chang W C, Olivares S, et al. Biophotonic cytotoxicity assay for high-throughput screening of cytolytic killing. Journal of immunological methods 2005 February; 297(1-2): 39-52.
43. Brentjens R J, Santos E, Nikhamin Y, Yeh R, Matsushita M, La Perle K, et al. Genetically targeted T cells eradicate systemic acute lymphoblastic leukemia xenografts. Clinical Cancer Research 2007; 13(18): 5426-5435.
44. Galanis A, Ma H, Rajkhowa T, Ramachandran A, Small D, Cortes J, et al. Crenolanib is a potent inhibitor of FLT3 with activity against resistance-conferring point mutants. Blood 2014; 123(1): 94-100.
45. Chen J, Schmitt A, Chen B, Rojewski M, Rubeler V, Fei F, et al. Nilotinib hampers the proliferation and function of CD8+T lymphocytes through inhibition of T cell receptor signalling. Journal of cellular and molecular medicine 2008; 12(5b): 2107-2118.
46. Fei F, Yu Y, Schmitt A, Rojewski M T, Chen B, Greiner J, et al. Dasatinib exerts an immunosuppressive effect on CD8+ T cells specific for viral and leukemia antigens. Experimental hematology 2008; 36(10): 1297-1308.
47. Negi S, Small D, Brown P. Level of FLT3 Expression in Leukemia Cells Correlates with Specific Histone Modifications and the Presence or Absence of MLL Fusion Genes, Implicating Epigenetic Regulation of FLT3 Expression. Blood 2008; 112(11): 4469.

1. A composition for use in a method for the treatment of cancer in a patient, the composition comprising:
(a) A kinase inhibitor; and
(b) An FLT3-targeting agent;
wherein in the method, the composition is to be administered to the patient.

2. The composition of claim 1 for the use of claim 1, wherein the method is a method comprising adoptive immunotherapy.

3. The composition of claim 1 or 2 for the use of claim 1 or 2, wherein the FLT3-targeting agent is capable of binding to the extracellular domain of FLT3.

4. The composition of any of claims 1 to 3 for the use of any of claims 1 to 3, wherein the FLT3-targeting agent inhibits growth of cells expressing FLT3.

5. The composition of any of claims 1 to 4 for the use of any of claims 1 to 4, wherein the FLT3-targeting agent comprises a cell targeting FLT3.

6. The composition of claim 5 for use of claim 5, wherein the cell is a cell expressing a chimeric antigen receptor.

7. The composition of claim 6 for use of claim 6, wherein the chimeric antigen receptor is capable of binding to FLT3.

8. The composition of any of claims 5 to 7 for use of any of claims 5 to 7, wherein the cell is a cell selected from the group of T cells, NK cells, and B cells.

9. The composition of any of claims 5 to 8 for use of any of claims 5 to 8, wherein the cell is a T cell.

10. The composition of any of claims 6 to 9 for use of any of claims 6 to 9, wherein the chimeric antigen receptor comprises the sequence of SEQ ID NO: 2 or a sequence at least 90% identical thereto, or wherein the chimeric antigen receptor comprises the sequence of SEQ ID NO: 4 or a or a sequence at least 90% identical thereto.

11. The composition of claim 10 for use of claim 10, wherein the chimeric antigen receptor comprises the sequence of SEQ ID NO: 2, or a sequence at least 90% identical thereto.

12. The composition of claim 10 for use of claim 10, wherein the chimeric antigen receptor comprises the sequence of SEQ ID NO: 4, or a sequence at least 90% identical thereto.

13. The composition of any of claims 6 to 12 for use of any of claims 6 to 12, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto, or wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.

14. The composition of claim 13 for use of claim 13, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto.

15. The composition of claim 13 for use of claim 13, wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.

16. The composition of any of claims 1 to 4 for the use of any of claims 1 to 4, wherein the FLT3-targeting agent comprises a protein.

17. The composition of claim 16 for the use of claim 16, wherein the protein is an antibody or fragment thereof capable of binding to FLT3.

18. The composition of claim 17 for the use of claim 17, wherein the antibody or fragment thereof comprises a heavy chain variable domain sequence of SEQ ID NO: 5 or a sequence at least 90% identical thereto and a light chain variable domain sequence of SEQ ID NO: 6 or a sequence at least 90% identical thereto, or wherein the antibody or fragment thereof comprises a heavy chain variable domain sequence of SEQ ID NO: 7 or a sequence at least 90% identical thereto and a light chain variable domain sequence of SEQ ID NO: 8 or a sequence at least 90% identical thereto.

19. The composition of claim 18 for the use of claim 18, wherein the antibody is an antibody comprising a heavy chain variable domain which comprises the amino acid sequence of SEQ ID NO: 5, and a light chain variable domain which comprises the amino acid sequence of SEQ ID NO: 6.

20. The composition of claim 18 for the use of claim 18, wherein the antibody is an antibody comprising a heavy chain variable domain which comprises the amino acid sequence of SEQ ID NO: 7, and a light chain variable domain which comprises the amino acid sequence of SEQ ID NO: 8.

21. The composition of any of claims 1 to 20 for the use of any of claims 1 to 20, wherein the kinase inhibitor is a multikinase inhibitor.

22. The composition of any of claims 1 to 21 for the use of any of claims 1 to 21, wherein the kinase inhibitor is a tyrosine kinase inhibitor.

23. The composition of any of claims 1 to 22 for the use of any of claims 1 to 22, wherein the kinase inhibitor is an FLT3 inhibitor.

24. The composition of any of claims 1 to 23 for the use of any of claims 1 to 23, wherein the kinase inhibitor is a kinase inhibitor capable of causing upregulation of FLT3 in said cancer.

25. The composition of any of claims 1 to 24 for the use of any of claims 1 to 24, wherein the kinase inhibitor is a kinase inhibitor capable of causing upregulation of FLT3 cell surface expression in said cancer.

26. The composition of any of claims 1 to 25 for the use of any of claims 1 to 25, wherein the kinase inhibitor is a kinase inhibitor capable of causing upregulation of mutated FLT3 in said cancer.

27. The composition of any of claims 1 to 26 for the use of any of claims 1 to 26, wherein the kinase inhibitor does not cause upregulation of wild-type FLT3 in said cancer.

28. The composition of claim 26 for the use of claim 26, wherein the mutated FLT3 comprises a mutated tyrosine kinase domain, and/or wherein the mutated FLT3 comprises internal tandem duplications.

29. The composition of claim 28 for the use of claim 28, wherein the mutated FLT3 comprises internal tandem duplications.

30. The composition of claim 28 for the use of claim 28, wherein the mutated FLT3 comprises a mutated tyrosine kinase domain.

31. The composition of any of claims 1 to 30 for the use of any of claims 1 to 30, wherein the kinase inhibitor does not inhibit T cells expressing chimeric antigen receptors.

32. The composition of any of claims 1 to 31 for the use of any of claims 1 to 31, wherein the kinase inhibitor is a type I or a type II FLT3 inhibitor.

33. The composition of claim 32 for the use of claim 32, wherein the kinase inhibitor is a type I FLT3 inhibitor.

34. The composition of claim 32 for the use of claim 32, wherein the kinase inhibitor is a type II FLT3 inhibitor.

35. The composition of any of claims 1 to 32 for the use of any of claims 1 to 32, wherein the kinase inhibitor is selected from the group consisting of crenolanib, midostaurin, and quizartinib.

36. The composition of claim 35 for the use of claim 35, wherein the kinase inhibitor is crenolanib.

37. The composition of claim 35 for the use of claim 35, wherein the kinase inhibitor is quizartinib.

38. The composition of claim 35 for the use of claim 35, wherein the kinase inhibitor is midostaurin.

39. The composition of any of claims 1 to 38 for the use of any of claims 1 to 38, wherein said treatment of cancer has an improved clinical outcome compared to a monotherapeutic treatment with either said FLT3-targeting agent or said kinase inhibitor alone.

40. The composition of any of claims 1 to 39 for the use of any of claims 1 to 39, wherein the FLT3-targeting agent and the kinase inhibitor prolong the progression free survival of the patient compared to monotherapy with either said FLT3-targeting agent or said kinase inhibitor alone.

41. The composition of any of claims 5 to 40 for the use of any of claims 5 to 40, wherein the cell produces effector cytokines when administered to the patient.

42. The composition of claim 41 for the use of claim 41, wherein the cytokines are IFN-gamma and IL-2.

43. The composition of any of claims 1 to 42 for the use of any of claims 1 to 42, wherein said cancer is leukemia or lymphoma.

44. The composition of claim 43 for the use of claim 43, wherein said cancer is leukemia.

45. The composition of claim 44 for the use of claim 44, wherein said leukemia is mixed-lineage leukemia or acute lymphoblastic leukemia.

46. The composition of claim 44 for the use of claim 44, wherein said leukemia is acute myeloid leukemia.

47. The composition of any of claims 1 to 46 for the use of any of claims 1 to 46, wherein the method is a method wherein the number of FLT3 molecules on the cell surface is increased, preferably wherein the number of FLT3 molecules on the cell surface is increased in the cancer cells.

48. The composition of claim 47 for the use of claim 47, wherein the FLT3 upregulation is caused by treatment with said kinase inhibitor.

49. The composition of claim 48 for the use of claim 48, wherein the cancer has acquired a resistance to a monotherapeutic treatment with said kinase inhibitor or wherein the cancer has acquired a resistance to a monotherapeutic treatment with said kinase inhibitor in combination with chemotherapy.

50. The composition of any of claims 1 to 49 for the use of any of claims 1 to 49, wherein the cancer expresses wild-type FLT3.

51. The composition of any of claims 1 to 49 for the use of any of claims 1 to 49, wherein the cancer expresses mutated FLT3.

52. The composition of claim 51 for the use of claim 51, wherein the mutated FLT3 is mutationally activated.

53. The composition of any of claim 51 or 52 for the use of any of claim 51 or 52, wherein the mutated FLT3 is mutated in the tyrosine kinase domain.

54. The composition of any of claims 51 to 53 for the use of any of claims 51 to 53, wherein the mutated FLT3 comprises internal tandem duplications.

55. The composition of any of claims 1 to 54 for the use of any of claims 1 to 54, wherein the treatment is a first-line therapy.

56. The composition of any of claims 1 to 54 for the use of any of claims 1 to 54, wherein the treatment is a second-line therapy, a third-line therapy, or a fourth-line therapy.

57. A chimeric antigen receptor capable of binding FLT3.

58. The chimeric antigen receptor of claim 57, wherein the chimeric antigen receptor comprises an IgG4-Fc hinge spacer, a CD28 transmembrane and costimulatory domain, and a CD3z signaling domain.

59. The chimeric antigen receptor of any of claim 57 or 58, wherein the chimeric antigen receptor comprises the sequence of SEQ ID NO: 2 or a sequence at least 90% identical thereto, or wherein the chimeric antigen receptor comprises the sequence of SEQ ID NO: 4 or a sequence at least 90% identical thereto.

60. The chimeric antigen receptor of claim 59, wherein the chimeric antigen receptor comprises the sequence of SEQ ID NO: 2 or a sequence at least 90% identical thereto.

61. The chimeric antigen receptor of claim 59, wherein the chimeric antigen receptor comprises the sequence of SEQ ID NO: 4 or a sequence at least 90% identical thereto.

62. The chimeric antigen receptor of any of claim 57 or 58, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto, or wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.

63. The chimeric antigen receptor of claim 62, wherein the chimeric antigen receptor comprises a heavy chain variable domain sequence of SEQ ID NO: 5 or a sequence at least 90% identical thereto and a light chain variable domain sequence of SEQ ID NO: 6 or a sequence at least 90% identical thereto.

64. The chimeric antigen receptor of claim 62, wherein the chimeric antigen receptor comprises a heavy chain variable domain sequence of SEQ ID NO: 7 or a sequence at least 90% identical thereto and a light chain variable domain sequence of SEQ ID NO: 8 or a sequence at least 90% identical thereto.

65. A cell comprising the chimeric antigen receptor of any one of claims 57 to 64.

66. The cell of claim 65, wherein the cell expressing the chimeric antigen receptor is obtainable by expressing the chimeric antigen receptor through stable gene transfer.

67. The cell of claim 65, wherein the cell expressing the chimeric antigen receptor is obtainable by expressing the chimeric antigen receptor through transient gene transfer.

68. The cell of any of claims 65 to 67, wherein the cell is a cell selected from the group of T cells, NK cells, and B cells.

69. The cell of claim 68, wherein the cell is a T cell.

70. The cell of any of claims 65 to 69, wherein the cell is CD8 positive.

71. The cell of any of claims 65 to 70, wherein the cell is CD4 positive.

72. An FLT3-targeting agent for use in a method of treating cancer.

73. The FLT3-targeting agent of claim 72 for the use of claim 72, wherein the method of treating cancer is a method of treating cancer with a kinase inhibitor.

74. The FLT3-targeting agent of any of claim 72 or 73 for the use of any of claim 72 or 73, wherein the FLT3-targeting agent is an FLT3-targeting agent as defined in any one of claims 3 to 20.

75. The FLT3-targeting agent of any of claims 72 to 74 for the use of any of claims 72 to 74, wherein the kinase inhibitor is a kinase inhibitor as defined in any one of claims 21 to 38.

76. The FLT3-targeting agent of any of claims 72 to 75 for the use of any of claims 72 to 75, wherein the cancer is a cancer as defined any one of claims 43-54.

77. The FLT3-targeting agent of any of claims 72 to 76 for the use of any of claims 72 to 76, wherein the use is a use as defined in any one of claims 1-56.

78. The FLT3-targeting agent of any of claims 72 to 77 for the use of any of claims 72 to 77, wherein the kinase inhibitor is to be administered at least once or multiple times prior to administering the FLT3-targeting agent, concurrently to administering the FLT3-targeting agent, or after administering the FLT3-targeting agent.

79. The FLT3-targeting agent of claim 78 for the use of claim 78, wherein the kinase inhibitor is to be administered at least once or multiple times prior to administering the FLT3-targeting agent.

80. The FLT3-targeting agent of claim 78 for the use of claim 78, wherein the kinase inhibitor is to be administered at least once or multiple times concurrently to administering the FLT3-targeting agent.

81. The FLT3-targeting agent of claim 78 for the use of claim 78, wherein the kinase inhibitor is to be administered at least once or multiple times after administering the FLT3-targeting agent.

82. A kit comprising an FLT3-targeting agent and a kinase inhibitor.

83. The kit according to claim 82, wherein the FLT3-targeting agent is an FLT3-targeting agent as defined in any one of claims 3-20.

84. The kit according to any of claim 82 or 83, wherein the kinase inhibitor is a kinase inhibitor as defined in any one of claims 21-38.

85. The kit according to any of claims 82 to 84, wherein said FLT3-targeting agent further comprises a pharmaceutical acceptable carrier.

86. The kit according to any of claims 82 to 85, wherein said kinase inhibitor further comprises a pharmaceutical acceptable carrier.

87. A composition comprising:
(a) A kinase inhibitor; and
(b) An FLT3-targeting agent.

88. The composition of claim 87, wherein the FLT3-targeting agent is an FLT3-targeting agent as defined in any one of claims 3-20.

89. The composition of any of claim 87 or 88, wherein the kinase inhibitor is kinase inhibitor as defined in any one of claims 21-38.

90. The composition of any of claims 87 to 89, further comprising a pharmaceutically acceptable carrier.

91. The composition of any of claims 87 to 90, wherein the composition is suitable for treating cancer.

92. The composition of claim 91, wherein the cancer is a cancer as defined in any one of claims 43-54.

93. A combination of the FLT3-targeting agent as defined in claim 72 and a kinase inhibitor.

94. The combination of claim 93 for use in a method for the treatment of cancer in a patient.

95. The combination of claim 93 or the combination for use of claim 94, wherein the FLT3-targeting agent is an FLT3-targeting agent as defined in any one of claims 3-20.

96. The combination of claim 93 or the combination for use of any of claims 94 to 95, wherein the kinase inhibitor is kinase inhibitor as defined in any one of claims 21-38.

97. The combination of claim 93 or the combination for use of any of claims 94 to 96, wherein the cancer is a cancer as defined in any one of claims 43-54.

98. The combination of claim 93 or the combination for use of any of claims 94 to 97, wherein the use is a use as defined in any one of claims 1-56.

99. A combination of FLT3 CAR-T cells and a kinase inhibitor, for use in a method for the treatment of cancer, wherein the combination is to be administered prior to or after an allogeneic hematopoietic stem cell transplantation to treat the cancer.

100. The combination for use according to claim 99, wherein the FLT3 CAR-T cells are autologous FLT3 CAR-T cells.

101. The combination for use according to claim 99, wherein the FLT3 CAR-T cells are allogeneic FLT3 CAR-T cells.

102. The combination for use according to any one of claims 99 to 101, wherein the cancer is a cancer as defined in any one of claims 43-54.

103. The combination for use according to any one of claims 99 to 102, wherein the cancer is FLT3-ITD+AML.

104. The combination for use according to any one of claims 99 to 103, wherein the kinase inhibitor is as defined in any one of claims 21-38.

105. The combination for use according to any one of claims 99 to 104, wherein the kinase inhibitor is crenolanib.
